(19) United States
(12) Patent Application Publication
(10) Pub. No.: US 2023/0250166 A1
(43) Pub. Date: Aug. 10, 2023

(54) ANTI-ADRENOMEDULLIN (ADM) BINDER FOR USE IN THERAPY OF PATIENTS IN SHOCK

(71) Applicant: ADRENOMED AG, Hennigsdorf (DE)

(72) Inventor: Andreas BERGMANN, Berlin (DE)

(73) Assignee: ADRENOMED AG, Hennigsdorf (DE)

(21) Appl. No.: 17/802,817

(22) PCT Filed: Mar. 1, 2021
(86) PCT No.: PCT/EP2021/055068

§ 371 Date: Aug. 26, 2022

(30) Foreign Application Priority Data
Feb. 27, 2020 (EP) 20159913.1
Nov. 6, 2020 (EP) 20206317.8

(51) Int. Cl. C07K 16/26

(52) U.S. Cl. CPC C07K 16/26; A61K 2039/545

(57) ABSTRACT

Subject matter of the present invention is an anti-adrenomedullin (ADM) antibody or an anti-adrenomedullin antibody fragment or anti-ADM non-Ig scaffold for use in therapy of patients in shock and/or for use in therapy of diseases which necessitates admission of the patients to ICU.

The instant application contains a Sequence Listing which has been submitted electronically in ASCII format and is hereby incorporated by reference in its entirety. Said ASCII copy, created on Apr. 26, 2023, is named BOEHMERP-0337_SL.txt and is 25,443 bytes in size.

Subject matter of the present invention is an anti-adrenomedullin (ADM) antibody or an anti-adrenomedullin antibody fragment or anti-ADM non-Ig scaffold for use in therapy of patients in shock and/or for use in therapy of diseases which necessitates admission of the patients to ICU.

### BACKGROUND

Diverse diseases or illnesses may have common, partially non-specific symptoms that can range from unpleasant to unbearable for the individual suffering from therefrom. Quite often individuals experiencing more than one symptom need to take several drugs to experience alleviation of these symptoms. There is an ongoing need for new forms of therapy or prevention of symptoms associated with many different underlying diseases or illnesses. In particular, it would be helpful to provide medicaments or drugs that can be used in the therapy or prevention of more than one symptom associated with an underlying disease or illness. The present invention addresses this need.

The peptide adrenomedullin (ADM) was described for the first time in 1993 (Kitamura K. et al. 1993. Biochemical and Biophysical Research Communications Vol. 192 (2): 553-560) as a novel hypotensive peptide comprising 52 amino acids, which had been isolated from a human pheochromocytome. In the same year, cDNA coding for a precursor peptide comprising 185 amino acids and the complete amino acid sequence of this precursor peptide were also described. The precursor peptide, which comprises, inter alia, a signal sequence of 21 amino acids at the N-terminus, is referred to as “preproadrenomedullin” (pre-proADM). In the present description, all amino acid positions specified usually relate to the pre-proADM which comprises the 185 amino acids. The peptide adrenomedullin (ADM) is a peptide which comprises 52 amino acids (SEQ ID NO: 1) and which comprises the amino acids 95 to 146 of pre-proADM, from which it is formed by proteolytic cleavage. To date, only a few fragments of the peptide fragments formed in the cleavage of the pre-proADM have been more exactly characterized, in particular the physiologically active peptides adrenomedullin (ADM) and “PAMP”, a peptide comprising 20 amino acids (22-41) which follows the 21 amino acids of the signal peptide in pre-proADM. The discovery and characterization of ADM in 1993 triggered intensive research activity, the results of which have been summarized in various review articles, in the context of the present description, reference being made in particular to the articles to be found in an issue of “Peptides” devoted to ADM in particular (Editorial, Takahashi K. 2001. Peptides 22:1691) and (Eto T. 2001. Peptides 22: 1693-1711). A further review is (Hinson et al. 2000. Endocrine Reviews 21(2):138-167). In the scientific investigations to date, it has been found, inter alia, that ADM may be regarded as a poly-functional regulatory peptide. It is released into the circulation in an inactive form extended by glycine (Kitamura K. et al. 1998. Biochem. Biophys. Res. Commun. 244(2):551-555). There is also a binding protein (Pio R. et al. 2001. The Journal of Biological Chemistry 276(15):12292-12300) which is specific for ADM and probably likewise modulates the effect of ADM. Those physiological effects of ADM as well as of PAMP which are of primary importance in the investigations to date were the effects influencing blood pressure.

ADM is an effective vasodilator, and it is possible to associate the hypotensive effect with the particular peptide segments in the C-terminal part of ADM. It has furthermore been found that the above-mentioned further physiologically active peptide PAMP formed from pre-proADM likewise exhibits a hypotensive effect, even if it appears to have an action mechanism differing from that of ADM.

It has furthermore been found that the concentrations of ADM, which can be measured in the circulation and other biological liquids are, in a number of pathological states, significantly above the concentrations to be found in healthy control persons. Thus, the ADM level in patients with congestive heart failure, myocardial infarction, kidney diseases, hypertensive disorders, diabetes mellitus, in the acute phase of shock and in sepsis and septic shock are significantly increased, although to different extents. The PAMP concentrations are also increased in some of said pathological states, but the plasma levels are lower relative to ADM ((Eto, T., supra). It is furthermore known that unusually high concentrations of ADM are to be observed in sepsis, and the highest concentrations in septic shock (cf. (Eto, T., “supra) and (Hirata et al. Journal of Clinical Endocrinology and Metabolism 1996. 81(4): 1449-1453; Ehlenz K. et al. 1997. Exp Clin Endocrinol Diabetes 105: 156-162); Tomoda Y. et al. 2001. Peptides 22: 1783-1794; Ueda S. et al. 1999. Am. J. Respir. Crit. Care Med. 160: 132-136; and Wang P. Peptides 2001. 22: 1835-1840).

WO-A1 2004/097423 describes the use of an antibody against adrenomedullin for diagnosis, prognosis, and treatment of cardiovascular disorders. Treatment of diseases by blocking the ADM receptor are also described in the art, (e.g. WO-A1 2006/027147, PCT/EP2005/012844) said diseases may be sepsis, septic shock, cardiovascular diseases, infections, dermatological diseases, endocrinological diseases, metabolic diseases, gastroenterological diseases, cancer, inflammation, hematological diseases, respiratory diseases, muscle skeleton diseases, neurological diseases, urological diseases. It is reported for the early phase of sepsis that ADM improves heart function and the blood supply in liver, spleen, kidney and small intestine. ADM-neutralizing antibodies neutralize the before mentioned effects during the early phase of sepsis (Wang, P., “Adrenomedullin and cardiovascular responses in sepsis”, Peptides, Vol. 22, pp. 1835-1840 (2001).

For other diseases blocking of ADM may be beneficial to a certain extent. However, it might also be detrimental if ADM is totally neutralized, as a certain amount of ADM may be required for several physiological functions. In many reports it was emphasized, that the administration of ADM may be beneficial in certain diseases. In contrast thereto, in other reports ADM was reported as being life threatening when administered in certain conditions.

Administration of ADM in combination with ADM-binding-Protein-1 is described for treatment of sepsis and septic shock in the art. It is assumed that treatment of septic animals with ADM and ADM-binding-Protein-1 prevents transition to the late phase of sepsis. It has to be noted that in a living organism ADM binding protein (complement factor H) is present in the circulation of said organism in high concentrations (Pio et al.: Identification, characterization, and physiological actions of factor H as an Adrenomedullin binding Protein present in Human Plasma; Microscopy Res. and Technique, 55:23-27 (2002) and Martinez et al.; Mapping of the Adrenomedullin-Binding domains in Human Complement factor H; Hypertens Res Vol. 26, Suppl (2003), S56-59).

The efficacy of non-neutralizing antibody targeted against the N-terminus of ADM was investigated in a survival study in CLP-induced sepsis in mice. Pre-treatment with the non-neutralizing antibody resulted in decreased catecholamine infusion rates, kidney dysfunction, and ultimately improved survival (Struck et al. 2013. Intensive Care Med Exp 1(1):22; Wagner et al. 2013. Intensive Care Med Exp 1(1):21).

Due to these positive results, a humanized version of an N-terminal anti-ADM antibody, named Adrecizumab, has been developed for further clinical development. Beneficial effects of Adrecizumab on vascular barrier function and survival were recently demonstrated in preclinical models of systemic inflammation and sepsis (Geven et al. 2018. Shock 50(6):648-654). In this study, pre-treatment with Adrecizumab attenuated renal vascular leakage in endotoxemic rats as well as in mice with CLP-induced sepsis, which coincided with increased renal expression of the protective peptide Ang-1 and reduced expression of the detrimental peptide vascular endothelial growth factor. Also, pre-treatment with Adrecizumab improved 7-day survival in CLP-induced sepsis in mice from 10 to 50% for single and from 0 to 40% for repeated dose administration. Of particular interest is the proposed mechanism of action of Adrecizumab. Both animal and human data reveal a potent, dose-dependent increase of circulating ADM following administration of this antibody. Based on pharmacokinetic data and the lack of an increase in MR-proADM (an inactive peptide fragment derived from the same prohormone as ADM), the higher circulating ADM levels cannot be explained by an increased production.

A mechanistic explanation for this increase could be that the excess of antibody in the circulation may drain ADM from the interstitium to the circulation, since ADM is small enough to cross the endothelial barrier, whereas the antibody is not (Geven et al. 2018. Shock. 50(2):132-140; and Voors et al (J. Eur J Heart Fail. 2019 February; 21(2):163-171)). In addition, binding of the antibody to ADM leads to a prolongation of ADM's half-life. Even though NT-ADM antibodies partially inhibit ADM-mediated signalling, a large increase of circulating ADM results in an overall “net” increase of ADM activity in the blood compartment, where it exerts beneficial effects on ECs (predominantly barrier stabilization), whereas ADMs detrimental effects on VSMCs (vasodilation) in the interstitium are reduced.

However, it is one object of the invention to identify the best timing for administering the antibody directed to ADM in order for the patients to optimally benefit from the above mechanism.

WO2013/072510 describes a non-neutralizing anti-ADM antibody for use in therapy of a severe chronical or acute disease or acute condition of a patient for the reduction of the mortality risk for said patient.

WO2013/072511 describes a non-neutralizing anti-ADM antibody for use in therapy of a chronical or acute disease or acute condition of a patient for prevention or reduction of organ dysfunction or organ failure.

WO2013/072512 describes a non-neutralizing anti-ADM antibody that is an ADM stabilizing antibody that enhances the half-life (t_{1/2} half retention time) of adrenomedullin in serum, blood, plasma. This ADM stabilizing antibody blocks the bioactivity of ADM to less than 80%.

WO2013/072513 shows that in patients having a chronic or acute disease or acute condition in need for stabilizing the circulation, anti-Adrenomedullin (ADM) antibody or an anti-adrenomedullin antibody fragment or an anti-ADM non-Ig scaffold stabilizes the blood circulation of patients and reduces the vasopressor requirement, e.g. catecholamine of said patient.

WO2013/072514 shows anti-Adrenomedullin (ADM) antibody or an anti-adrenomedullin antibody fragment or an anti-ADM non-Ig scaffold can be efficiently used to regulate the fluid balance in a patient having a chronic or acute disease or acute condition, especially patients at the ICU (Intensive Care Unit) who suffers from fluid imbalance.

WO2017/182561 describes methods for determining the total amount or active DPP3 in a sample of a patient for the diagnosis of a disease related to necrotic processes. It further describes a method of treatment of necrosis-related diseases by antibodies directed to DPP3.

The inventors have now found that treatment of anti-adrenomedullin (ADM) antibody or an anti-adrenomedullin antibody fragment or an anti-ADM non-Ig scaffold can be particularly effective in therapy of a patient in shock, in particular septic shock, when administered within a certain period of time.

Thus, anti-ADM antibody or anti-ADM antibody fragment or anti-ADM non-Ig scaffold may be administered at a point of time when the patient is in need of anti-ADM treatment in particular after ICU admission. According to the invention said patient is treated at the optimal timing after shock and/or after ICU admission.

In addition to and/or in connection with the above effect, it is also the surprising finding of the present invention, that patients with shock will have most benefit of a therapy with an anti-ADM antibody if the level of DPP3 in a bodily fluid sample is below a threshold.

### Definitions

Before describing the invention in detail, it is deemed expedient to provide definitions for certain technical terms used throughout the description. Although the present invention will be described with respect to particular embodiments, this description is not to be construed in a limiting sense. Before describing in detail exemplary embodiments of the present invention, definitions important for understanding the present invention are given.

As used in this specification and in the appended claims, the singular forms of “a” and “an” also include the respective plurals unless the context clearly dictates otherwise.

In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%.

It is to be understood that the term “comprising” is not limiting. For the purposes of the present invention the term “consisting” of is considered to be a preferred embodiment of the term “comprising” of. If hereinafter a group is defined to comprise at least a certain number of embodiments, this is meant to also encompass a group, which preferably consists of these embodiments only.

It is also to be understood that the terminology used herein is for the purpose of describing particular embodiments only, and is not intended to limit the scope of the present invention that will be limited only by the appended claims.

In accordance with the invention the ADM-binding-Protein-1 may be also referred to as ADM-binding-Protein-1 (complement factor H).

As used herein, the term “shock” is characterized by decreased oxygen delivery and/or increased oxygen consumption or inadequate oxygen utilization leading to cellular and tissue hypoxia. It is a life-threatening condition of circulatory failure and most commonly manifested as hypotension (systolic blood pressure less than 90 mm Hg or MAP less than 65 mmHg). Shock is divided into four main types based on the underlying cause: hypovolemic, cardiogenic, obstructive, and distributive shock (Vincent and De Backer 2014. N. Engl. J. Med. 370(6): 583).

The term “cardiogenic shock” refers to shock where the patient may have suffered an acute coronary syndrome (e.g. acute myocardial infarction) or wherein said patient has heart failure (e.g. acute decompensated heart failure), myocarditis, arrhythmia, cardiomyopathy, valvular heart disease, aortic dissection with acute aortic stenosis, traumatic chordal rupture or massive pulmonary embolism. Cardiogenic shock (CS) is defined as a state of critical endorgan hypoperfusion due to reduced cardiac output. Notably, CS forms a spectrum that ranges from mild hypoperfusion to profound shock. Established criteria for the diagnosis of CS are: (i) systolic blood pressure, ≤90 mmHg for ≥30 min or vasopressors required to achieve a blood pressure≥90 mmHg; (ii) pulmonary congestion or elevated left-ventricular filling pressures; (iii) signs of impaired organ perfusion with at least one of the following criteria: (a) altered mental status; (b) cold, clammy skin; (c) oliguria (≤0.5 mL/kg/h or <30 mL/h); (d) increased serum-lactate (Reynolds and Hochman 2008. Circulation 117: 686-697). Acute myocardial infarction (AMI) with subsequent ventricular dysfunction is the most frequent cause of CS accounting for approximately 80% of cases. Mechanical complications such as ventricular septal (4%) or free wall rupture (2%), and acute severe mitral regurgitation (7%) are less frequent causes of CS after AMI. (Hochman et al. 2000. J Am Coll Cardiol 36: 1063-1070). Non-AMI-related CS may be caused by decompensated valvular heart disease, acute myocarditis, arrhythmias, etc. with heterogeneous treatment options. This translates in 40 000 to 50 000 patients per year in the USA and 60 000 to 70 000 in Europe. Despite advances in treatment mainly by early revascularization with subsequent mortality reduction, CS remains the leading cause of death in AMI with mortality rates still approaching 40-50% according to recent registries and randomized trials (Goldberg et al. 2009. Circulation 119: 1211-1219).

The term “hypovolemic shock” refers to a shock where the patient may have suffered a hemorrhagic disease including gastrointestinal bleed, trauma, vascular etiologies (e.g. ruptured abdominal aortic aneurysm, tumor eroding into a major blood vessel) and spontaneous bleeding in the setting of anticoagulant use or a non-hemorrhagic disease including vomiting, diarrhea, renal loss, skin losses/insensible losses (e.g. burns, heat stroke) or third-space loss in the setting of pancreatitis, cirrhosis, intestinal obstruction, trauma. Hypovolemic shock is characterized by decreased intravascular volume and can be divided into two broad subtypes: hemorrhagic and non-hemorrhagic. Common causes of hemorrhagic hypovolemic shock include gastrointestinal bleed, trauma, vascular etiologies (e.g. ruptured abdominal aortic aneurysm, tumor eroding into a major blood vessel) and spontaneous bleeding in the setting of anticoagulant use. Common causes of non-hemorrhagic hypovolemic shock include vomiting, diarrhea, renal loss, skin losses/insensible losses (e.g. burns, heat stroke) or third-space loss in the setting of pancreatitis, cirrhosis, intestinal obstruction, trauma. For review see Kova and Paul 2018. Shock. StatPearls [Internet]. Treasure Island (FL): StatPearls Publishing; 2019-2018 Oct. 27.

The term “obstructive shock” refers to a shock where the patient may have suffered a cardiac tamponade, tension pneumothorax, pulmonary embolism or aortic stenosis. Obstructive shock is due to a physical obstruction of the great vessels or the heart itself. Several conditions can result in this form of shock (e.g. cardiac tamponade, tension pneumothorax, pulmonary embolism, aortic stenosis). For review see Koya and Paul 2018. Shock. StatPearls [Internet]. Treasure Island (FL): StatPearls Publishing; 2019-2018 Oct. 27.

The term “distributive shock” refers to a shock where the patient may have septic shock, neurogenic shock, anaphylactic shock or shock due to adrenal crisis. According to the cause, there are four types of distributive shock: neurogenic shock (decreased sympathetic stimulation leading to decreased vasal tone), anaphylactic shock, septic shock and shock due to adrenal crisis. In addition to sepsis, distributive shock can be caused by systemic inflammatory response syndrome (SIRS) due to conditions other than infection such as pancreatitis, burns or trauma. Other causes include, toxic shock syndrome (TSS), anaphylaxis (a sudden, severe allergic reaction), adrenal insufficiency (acute worsening of chronic adrenal insufficiency, destruction or removal of the adrenal glands, suppression of adrenal gland function due to exogenous steroids, hypopituitarism and metabolic failure of hormone production), reactions to drugs or toxins, heavy metal poisoning, hepatic (liver) insufficiency and damage to the central nervous system. For review see Kova and Paul 2018. Shock. StatPearls [Internet]. Treasure Island (FL): StatPearls Publishing; 2019-2018 Oct. 27.

Refractory shock has been defined as requirement of noradrenaline infusion of >0.5 μg/kg/min despite adequate volume resuscitation. Mortality in these patients may be as high as 94% and the assessment and management of these patients requires a much more aggressive approach for survival. The term “refractory shock” is used when the tissue perfusion cannot be restored with the initial corrective measures employed (e.g. vasopressors) and may therefore be referred to as “high vasopressor-dependent” or “vasopressor-resistant” shock (Udupa and Shetty 2018. Indian J Respir Care 7: 67-72). Patients with refractory shock may have features of inadequate perfusion such as hypotension (mean arterial blood pressure<65 mmHg), tachycardia, cold peripheries, prolonged capillary refill time, and tachypnea consequent to the hypoxia and acidosis. Fever may be seen in septic shock. Other signs of hypoperfusion such as altered sensorium, hyperlactatemia, and oliguria may also be seen. These well-known signs of shock are not helpful in identifying whether the problem is at the pump (heart) or circuitry (vessels and tissues). Different types of shock can coexist, and all forms of shock can become refractory, as evidenced by unresponsiveness to high-dose vasopressors (Udupa and Shetty 2018. Indian J Respir Care 7: 67-72).

Preferred examples of shock are shock due to hypovolemia, cardiogenic shock, obstructive shock and distributive shock, in particular cardiogenic shock, septic shock, shock due to Covid-19, shock due to burns and traumatic shock. These examples are defined in more details below.

As used herein, the term “admission to ICU” refers to patients admitted to intensive care are patients who have, or are likely to have, one or several acute, directly life-threatening malfunctions which require the use of organ supporting methods. Criteria for admitting patients to intensive care units have been developed which are well documented in the art (Nates et al. (2016), Critical care medicine 44: 1553-1602). The term “admission to ICU” shall encompass admissions under these criteria.

Types of organ support include:
Respiratory support therapy:
   comprising advanced respiratory support therapy such as tracheal intubation and mechanical ventilation support
   basic respiratory support therapy such as use of supplemental oxygen, the use of an incentive spirometer, chest percussion nebulization, etc.
Circulatory support therapy, such as mechanical circulatory support (e.g. the use of a intra-aortic balloon pump and a ventricular assist device) and the medical therapy including the use of an angiotensin converting enzyme, beta blockers, etc.
Renal support therapy such as hemodialysis and peritoneal dialysis,
Hemodynamic monitoring or support therapy such as measuring the pressure, flow and oxygen content of the blood, fluid resuscitation or blood transfusion and the use of vasoactive drugs 8e.g. nitroglycerine, nitric oxide, etc.),
Neurological monitoring or support such as an intraventricular catheter.

The extracorporeal organ support is described in detail for example in ICU Management & Practice, Volume 18—Issue 1, 2018.

In the following clinical criteria for SIRS, sepsis, severe sepsis, septic shock will be defined.

1) Systemic Inflammatory Host Response (SIRS) Characterized by at Least Two of the Following Symptoms
patients exhibit hypotension (mean arterial pressure is <65 mm Hg)
elevated serum lactate level being >4 mmol/L
blood glucose>7.7 mmol/L (in absence of diabetes)
central venous pressure is not within the range 8-12 mm Hg
urine output is <0.5 mL×kg⁻¹×hr⁻¹
central venous (superior vena cava) oxygen saturation is <70% or mixed venous<65%
heart rate is >90 beats/min
temperature<36° C. or >38° C.
respiratory rate>20/min
white cell count<4 or >12×10⁹/L (leucocytes); >10% immature neutrophils

2) Sepsis

Following at least two of the symptoms mentioned under 1), and additionally a clinical suspicion of new infection, being:
cough/sputum/chest pain
abdominal pain/distension/diarrhoea
line infection
endocarditis
dysuria
headache with neck stiffness
cellulitis/wound/joint infection
positive microbiology for any infection
3) Severe Sepsis

Provided that sepsis is manifested in patient, and additionally a clinical suspicion of any organ dysfunction, being:
blood pressure systolic<90/mean; <65 mmHG
lactate>2 mmol/L
Bilirubin>34 μmol/L
urine output<0.5 mL/kg/h for 2 h
creatinine>177 μmol/L
platelets<100×10⁹/L
SPO₂>90% unless O₂ given
4) Septic Shock

“Septic shock” refers to a potentially fatal medical condition that occurs when sepsis, which is organ injury or damage in response to infection, leads to dangerously low blood pressure and abnormalities in cellular metabolism. The Third International Consensus Definitions for Sepsis and Septic Shock (Sepsis-3) defines septic shock as a subset of sepsis in which particularly profound circulatory, cellular, and metabolic abnormalities are associated with a greater risk of mortality than with sepsis alone. Patients with septic shock can be clinically identified by a vasopressor requirement to maintain a mean arterial pressure of 65 mm Hg or greater and serum lactate level greater than 2 mmol/L (>18 mg/dL) in the absence of hypovolemia. This combination is associated with hospital mortality rates greater than 40% (Singer et al. 2016. JAMA. 315 (8): 801-10). The primary infection is most commonly caused by bacteria, but also may be by fungi, viruses or parasites. It may be located in any part of the body, but most commonly in the lungs, brain, urinary tract, skin or abdominal organs. It can cause multiple organ dysfunction syndrome (formerly known as multiple organ failure) and death. Frequently, people with septic shock are cared for in intensive care units. It most commonly affects children, immunocompromised individuals, and the elderly, as their immune systems cannot deal with infection as effectively as those of healthy adults. The mortality rate from septic shock is approximately 25-50%. “Septic shock” also refers to as a life-threatening organ dysfunction due to dysregulated host response to a proven or suspected infection which leads to at least a decline of mean arterial pressure (MAP)<65 mmHg, which is refractory to fluid resuscitation and requires vasopressors. Refractoriness to fluid resuscitation is defined as a lack of response to the administration of 30 mL of fluid per kilogram of body weight or is determined according to a clinician's assessment of inadequate hemodynamic results. In a septic shock according to the invention, at least one sign of end-organ dysfunction as mentioned under 3) above is manifested. Septic shock is indicated, if there is refractory hypotension that does not respond to treatment and intravenous fluid administration alone is insufficient to maintain a patient's blood pressure from becoming hypotensive also provides for an administration of an anti-ADM antibody or an anti-ADM antibody fragment or an anti-ADM non-Ig scaffold in accordance with the present invention.

Anti-Adrenomedullin (ADM) antibody is an antibody that binds specifically to ADM, Anti-adrenomedullin antibody fragment is a fragment of an anti-ADM antibody, wherein said fragment binds specifically to ADM. An anti-ADM non-Ig scaffold is a non-Ig scaffold that binds specifically to ADM.

In one embodiment the anti-ADM antibody or the anti-adrenomedullin antibody fragment or anti-ADM non-Ig scaffold according to the present invention reduces the vasopressor-agents requirement, e.g. catecholamine requirement, of said patient. The vasopressor-agents requirement, e.g catecholamine requirement of a patient is an indicator for the condition of the circulation of said patient. Thus, the anti-ADM antibody or the anti-adrenomedullin antibody fragment or anti-ADM non-Ig scaffold may be administered at a point of time when the patient is in need of a vasopressor agent, e.g. catecholamine.

In one embodiment of the invention said patient is a patient in shock in need of increasing the blood pressure.

Some patients with septic shock-induced hypofusion may remain hypotensive despite adequate fluid replacement. In these cases vasopressor agents are needed to increase MAP. Thus, in one embodiment of the invention the patient having a chronic or acute disease or acute condition is a patient in need of vasopressor agents to increase MAP. Catecholamines such as dopamine, epinephrine (adrenaline), norepinephrine (noradrenaline), and phenylephrine have been traditionally used to raise blood pressure in patients with septic shock. Recently also vasopressin has been suggested as potential vasopressor in patients in shock in need for stabilizing the circulation.

Vasopressor agents as catecholamine may stabilize the circulation of a patient having a chronic or acute disease or acute condition. In case the condition of the patient (low blood pressure) is very critical, vasopressor agents administration, e.g. catecholamine administration, alone may not prevent the break-down of the circulation. The additional administration of anti-ADM antibody or the anti-ADM antibody fragment or anti-ADM non-Ig scaffold together with administration of e.g. catecholamine may help to stabilize the circulation of a patient whose condition is so critical that catecholamine administration without administration of anti-ADM antibody or anti-ADM antibody fragment or anti-ADM non-Ig scaffold would not be sufficient in order to stabilize the circulation of said patient.

Further, vasopressors may have serious side effects. Dopamine stimulates D1 receptors in the renal regional circulation, producing vasodilation and increases blood flow. This is one of the reasons why clinicians have utilized low doses of dopamine to protect kidney function. Also for other vasopressors it has been suggested that increasing the blood pressure with certain drugs, despite its intuitive appeal as something beneficial, can be associated with worse outcomes.

Thus, subject of the invention is an anti-adrenomedullin (ADM) antibody or an anti-adrenomedullin antibody fragment or anti-ADM non-Ig scaffold for use in therapy of a patient in shock in order to replace the administration of a vasopressor totally or partially. This means the patient according to the present invention may be a patient being in need of or treatment with vasopressors or a patient receiving a treatment with vasopressors.

The circulation stabilizing effect of the anti-ADM antibody or the anti-ADM antibody fragment or anti-ADM non-Ig scaffold may thus be supporting the primary therapy of a shock. This means in one embodiment that the anti-ADM antibody or the anti-ADM antibody fragment or anti-ADM non-Ig scaffold is administered in addition to a first line treatment (primary therapy). In case of a shock e.g. septic shock or the like the primary therapy would be e.g. the administration of antibiotics. The anti-ADM antibody or the anti-ADM antibody fragment or anti-ADM non-Ig scaffold would stabilize the circulation and would help to prevent worsening of the critical condition of said patient until the e.g. antibiotic administration takes effect. As before mentioned the anti-ADM antibody or the anti-ADM antibody fragment or anti-ADM non-Ig scaffold may be administered in a preventive way or in a therapeutic way, this means in order to prevent circulation problems or in order to stabilize the circulation when circulation problems are present in a said patient.

It should be emphasized that the circulation problems comprised by the present invention may be acute circulation problems according to a specific embodiment of the invention.

In one embodiment of the invention an anti-Adrenomedullin (ADM) antibody or an anti-ADM antibody fragment or anti-ADM non-Ig scaffold is to be used in combination with vasopressors e.g. catecholamine wherein said combination is for use in therapy of a patient in shock for stabilizing the circulation of said patient.

In one embodiment of the invention said patient in shock being in need for stabilizing the circulation is characterized by the need of said patient to get administration of vasopressors e.g. catecholamine administration.

Subject matter of the invention in one specific embodiment is, thus, an anti-adrenomedullin (ADM) antibody or an anti-adrenomedullin antibody fragment binding to ADM or anti-ADM non-Ig scaffold binding to ADM for use in therapy of a patient in need of an administration of vasopressors, e.g. a catecholamine administration.

Furthermore, in one embodiment of the invention an anti-Adrenomedullin (ADM) antibody or an anti-adrenomedullin antibody fragment or an anti-ADM non-Ig scaffold is to be used in combination with fluids administered intravenously, wherein said combination is for use in therapy of a patient in shock for stabilizing the circulation of said patient. In one embodiment of the invention said patient having a shock and being in need for stabilizing the circulation is characterized by the need of said patient to get intravenous fluids.

Subject matter of the invention in one specific embodiment is, thus, an anti-Adrenomedullin (ADM) antibody or an anti-adrenomedullin antibody fragment or anti-ADM non-Ig scaffold for use in therapy of a patient in shock and in particular in need of intravenous fluids.

### DETAILED DESCRIPTION OF EMBODIMENTS OF THE INVENTION

Below, embodiments of the invention are provided. It is noted that generally embodiments can be combined with any other embodiment of the same category (product, process, use, method).

One embodiment of the invention relates to an adrenomedullin (ADM) antibody or an anti-adrenomedullin antibody fragment or anti-ADM non-Ig scaffold for use in therapy of a patient with shock, in particular septic shock, wherein said patient:
has suffered from shock, in particular from a septic shock not longer than 10 hours at the starting point of treatment with said Anti-adrenomedullin (ADM) antibody or an anti-adrenomedullin antibody fragment or anti-ADM non-Ig scaffold and/or
has been admitted to ICU not longer than 10 hours at the starting point of treatment with said Anti-adrenomedullin (ADM) antibody or an anti-adrenomedullin antibody fragment or anti-ADM non-Ig scaffold, and/or
has not received organ support at all or not longer than 10 hours of organ support at the starting point of treatment with said Anti-adrenomedullin (ADM) antibody or an anti-adrenomedullin antibody fragment or anti-ADM non-Ig scaffold, and
   wherein said antibody or fragment or scaffold binds to the N-terminal part (aa 1-21) of ADM: YRQSMNNFQGLRSFGCRFGTC (SEQ ID No. 4).

One embodiment of the invention relates to an adrenomedullin (ADM) antibody or an anti-adrenomedullin antibody fragment or anti-ADM non-Ig scaffold for use in therapy of a patient with shock, in particular septic shock, wherein said patient:
has suffered from shock, in particular from a septic shock not longer than 8.4 hours at the starting point of treatment with said Anti-adrenomedullin (ADM) antibody or an anti-adrenomedullin antibody fragment or anti-ADM non-Ig scaffold and/or
has been admitted to ICU not longer than 8.4 hours at the starting point of treatment with said Anti-adrenomedullin (ADM) antibody or an anti-adrenomedullin antibody fragment or anti-ADM non-Ig scaffold, and/or
has not received organ support at all or not longer than 8.4 hours of organ support at the starting point of treatment with said Anti-adrenomedullin (ADM) antibody or an anti-adrenomedullin antibody fragment or anti-ADM non-Ig scaffold, and
   wherein said antibody or fragment or scaffold binds to the N-terminal part (aa 1-21) of ADM: YRQSMNNFQGLRSFGCRFGTC (SEQ ID No. 4).

In a preferred embodiment, adrenomedullin (ADM) antibody or an anti-adrenomedullin antibody fragment or anti-ADM non-Ig scaffold is for use in therapy of a patient with shock, in particular septic shock, wherein said patient:
has suffered from shock, in particular from a septic shock no longer than 10 hours at the starting point of treatment with said Anti-adrenomedullin (ADM) antibody or an anti-adrenomedullin antibody fragment or anti-ADM non-Ig scaffold and/or
has been admitted to ICU not longer than 10 hours at the starting point of treatment with said Anti-adrenomedullin (ADM) antibody or an anti-adrenomedullin antibody fragment or anti-ADM non-Ig scaffold, and/or
has not received organ support at all or not longer than 10 hours of organ support at the starting point of treatment with said Anti-adrenomedullin (ADM) antibody or an anti-adrenomedullin antibody fragment or anti-ADM non-Ig scaffold, and
   wherein said antibody or fragment or scaffold binds to the N-terminal part (aa 1-21) of ADM: YRQSMNNFQGLRSFGCRFGTC (SEQ ID No. 4) and
   wherein, when the patient has suffered from shock, in particular from a septic shock a) not longer than 10 hours and has been admitted to ICU b) not longer than 10 hours, the starting point of treatment with said Anti-adrenomedullin (ADM) antibody or an anti-adrenomedullin antibody fragment or anti-ADM non-Ig scaffold is the shortest of a) and b).

In another embodiment, when the patient has been admitted to ICU a) not longer than 10 hours and c) has received no longer than 10 hours of organ support, the starting point of treatment with the Anti-adrenomedullin (ADM) antibody or an anti-adrenomedullin antibody fragment or anti-ADM non-Ig scaffold is the shortest of a) and c).

In another embodiment, when the patient has suffered from shock, in particular from a septic shock b) not longer than 10 hours and has received c) no longer than 10 hours of organ support, the starting point of treatment with the Anti-adrenomedullin (ADM) antibody or an anti-adrenomedullin antibody fragment or anti-ADM non-Ig scaffold is the shortest of b) and c).

In another embodiment, when the patient has suffered from shock, in particular from a septic shock a) not longer than 10 hours and the patient has been in shock b) not longer than 10 hours and has received c) no longer than 10 hours of organ support, the starting point of treatment with the Anti-adrenomedullin (ADM) antibody or an anti-adrenomedullin antibody fragment or anti-ADM non-Ig scaffold is the shortest of a), b) and c).

In another embodiment, the invention relates to an adrenomedullin (ADM) antibody or an anti-adrenomedullin antibody fragment or anti-ADM non-Ig scaffold for use in therapy of a patient with shock, in particular septic shock, wherein said patient:
has been in shock not longer than 10 hours at the starting point of treatment with said Anti-adrenomedullin (ADM) antibody or an anti-adrenomedullin antibody fragment or anti-ADM non-Ig scaffold and/or
has been admitted to ICU not longer than 10 hours at the starting point of treatment with said Anti-adrenomedullin (ADM) antibody or an anti-adrenomedullin antibody fragment or anti-ADM non-Ig scaffold, and/or
has not received organ support at all or not longer than 10 hours of organ support at the starting point of treatment with said Anti-adrenomedullin (ADM) antibody or an anti-adrenomedullin antibody fragment or anti-ADM non-Ig scaffold, and
wherein said antibody or fragment or scaffold binds to the N-terminal part (aa 1-21) of ADM: YRQSMNNFQGLRSFGCRFGTC (SEQ ID No. 4).

In a preferred embodiment. the patient has suffered from shock, in particular from a septic shock, not longer than 9, preferably 8.4, preferably 8.26 (0.344 days), preferably 8, preferably 7, preferably 6, preferably 5.76 (0.25 days), preferably 5.75 (0.24 days), 5, preferably 4, preferably 3 hours at the starting point of treatment with said Anti-adrenomedullin (ADM) antibody or an anti-adrenomedullin antibody fragment or anti-ADM non-Ig scaffold.

In a preferred embodiment. the patient has suffered from shock, in particular from a septic shock, not longer 8.4, preferably 8.26 (0.344 days) at the starting point of treatment with said Anti-adrenomedullin (ADM) antibody or an anti-adrenomedullin antibody fragment or anti-ADM non-Ig scaffold.

In a preferred embodiment, the patient has been admitted to ICU not longer than 9, preferably 8.4, preferably 8.26 (0.344 days), preferably 8, preferably 7, preferably 6, preferably 5.76 (0.25 days), preferably 5.75 (0.24 days) preferably 5, preferably 4, preferably 3 hours at the starting point of treatment with said Anti-adrenomedullin (ADM) antibody or an anti-adrenomedullin antibody fragment or anti-ADM non-Ig scaffold.

In a preferred embodiment, the patient has been admitted to ICU not longer than 8.4, preferably no longer than 8.26 (0.344 days), at the starting point of treatment with said Anti-adrenomedullin (ADM) antibody or an anti-adrenomedullin antibody fragment or anti-ADM non-Ig scaffold.

In a preferred embodiment. the patient has received organ support not longer than 9, preferably 8.4, preferably 8.26 (0.344 days), preferably 8, preferably 7, preferably 6, preferably 5.76 (0.25 days), preferably 5.75 (0.24 days), preferably 5, preferably 4, preferably 3 hours at the starting point of treatment with said Anti-adrenomedullin (ADM) antibody or an anti-adrenomedullin antibody fragment or anti-ADM non-Ig scaffold.

In a preferred embodiment. the patient has received organ support not longer than 8.4, preferably no longer than 8.26 (0.344 days at the starting point of treatment with said Anti-adrenomedullin (ADM) antibody or an anti-adrenomedullin antibody fragment or anti-ADM non-Ig scaffold In another embodiment. the anti-Adrenomedullin-adrenomedullin (ADM) antibody or an anti-adrenomedullin antibody fragment or anti-ADM non-Ig scaffold is for use in therapy of a patient suffering from shock, in particular septic shock, wherein said Anti-adrenomedullin (ADM) antibody or an anti-adrenomedullin antibody fragment or anti-ADM non-Ig scaffold is administered
within 10 hours after occurrence of shock in said patient and/or
within 10 hours after admission of said patient to ICU, and/or
before the patient has received organ support or not longer than 10 h of organ support, and
wherein said antibody or fragment or scaffold binds to the N-terminal part (aa 1-21) of ADM:

(SEQ ID No. 4)
YRQSMNNFQGLRSFGCRFGTC.

In another embodiment. the anti-Adrenomedullin-adrenomedullin (ADM) antibody or an anti-adrenomedullin antibody fragment or anti-ADM non-Ig scaffold is for use in therapy of a patient suffering from shock, in particular septic shock, wherein said Anti-adrenomedullin (ADM) antibody or an anti-adrenomedullin antibody fragment or anti-ADM non-Ig scaffold is administered
within 8.4 hours after occurrence of shock in said patient and/or
within 8.4 hours after admission of said patient to ICU, and/or
before the patient has received organ support or not longer than 10 h of organ support, and
wherein said antibody or fragment or scaffold binds to the N-terminal part (aa 1-21) of ADM:

(SEQ ID No. 4)
YRQSMNNFQGLRSFGCRFGTC.

In preferred embodiment. the anti-Adrenomedullin-adrenomedullin (ADM) antibody or an anti-adrenomedullin antibody fragment or anti-ADM non-Ig scaffold is for use in therapy of a patient suffering from shock, in particular septic shock, wherein said Anti-adrenomedullin (ADM) antibody or an anti-adrenomedullin antibody fragment or anti-ADM non-Ig scaffold is administered
within 10 hours after occurrence of shock in said patient and/or
within 10 hours after admission of said patient to ICU, and/or
before the patient has received organ support or not longer than 10 h of organ support, and
wherein said antibody or fragment or scaffold binds to the N-terminal part (aa 1-21) of ADM: YRQSMNNFQGLRSFGCRFGTC (SEQ ID No. 4), and
wherein, when the Anti-adrenomedullin (ADM) antibody or an anti-adrenomedullin antibody fragment or anti-ADM non-Ig scaffold can be administered a) within 10 hours after occurrence of shock in said patient and b) within 10 hours after admission of said patient to ICU, the Anti-adrenomedullin (ADM) antibody or an anti-adrenomedullin antibody fragment or anti-ADM non-Ig scaffold is administered at the shortest of a) and b).

In another embodiment, when the Anti-adrenomedullin (ADM) antibody or an anti-adrenomedullin antibody fragment or anti-ADM non-Ig scaffold can be administered a) within 10 hours after occurrence of shock in said patient and c) before the patient has received organ support or not longer than 10 h of organ support, the Anti-adrenomedullin (ADM) antibody or an anti-adrenomedullin antibody fragment or anti-ADM non-Ig scaffold is administered at the shortest of a) and c).

In another embodiment, when the Anti-adrenomedullin (ADM) antibody or an anti-adrenomedullin antibody fragment or anti-ADM non-Ig scaffold can be administered b) within 10 hours after admission of said patient to ICU and c) before the patient has received organ support or not longer than 10 h of organ support, the Anti-adrenomedullin (ADM) antibody or an anti-adrenomedullin antibody fragment or anti-ADM non-Ig scaffold is administered at the shortest of b) and c).

In another embodiment, when the Anti-adrenomedullin (ADM) antibody or an anti-adrenomedullin antibody fragment or anti-ADM non-Ig scaffold can be administered a) within 10 hours after occurrence of shock in said patient and b) within 10 hours after admission of said patient to ICU and c) before the patient has received organ support or not longer than 10 hours of organ support, the Anti-adrenomedullin (ADM) antibody or an anti-adrenomedullin antibody fragment or anti-ADM non-Ig scaffold is administered at the shortest of a), b) and c).

In another embodiment. the anti-Adrenomedullin-adrenomedullin (ADM) antibody or an anti-adrenomedullin antibody fragment or anti-ADM non-Ig scaffold is for use in therapy of a patient suffering from shock, in particular septic shock, wherein said Anti-adrenomedullin (ADM) antibody or an anti-adrenomedullin antibody fragment or anti-ADM non-Ig scaffold is administered
within 10 hours after occurrence of shock in said patient and/or
within 10 hours after admission of said patient to ICU, and/or
before the patient has received organ support or not longer than 10 hours of organ support, and
wherein said antibody or fragment or scaffold binds to the N-terminal part (aa 1-21) of ADM: YRQSMNNFQGLRSFGCRFGTC (SEQ ID No. 4).

In a preferred embodiment, the Anti-adrenomedullin (ADM) antibody or an anti-adrenomedullin antibody fragment or anti-ADM non-Ig scaffold is administered within 9, preferably 8.4, preferably 8.26 (0.344 days), preferably 8, preferably 7, preferably 6, preferably 5.76 (0.25 days), preferably 5.75 (0.25 days), preferably 5, preferably 4, preferably 3 hours after occurrence of shock and/or sepsis in said patient.

In a preferred embodiment, the Anti-adrenomedullin (ADM) antibody or an anti-adrenomedullin antibody fragment or anti-ADM non-Ig scaffold is administered within 8.4, preferably 8.26 (0.344 days) hours after occurrence of shock and/or sepsis in said patient.

In a preferred embodiment, the Anti-adrenomedullin (ADM) antibody or an anti-adrenomedullin antibody fragment or anti-ADM non-Ig scaffold is administered within 9, preferably 8.4, preferably 8.26 (0.344 days), preferably 8, preferably 7, preferably 6, preferably 5.76 (0.25 days), preferably 5.75 (0.25 days), preferably 5, preferably 4, preferably 3 hours after admission of said patient to ICU.

In a preferred embodiment, the Anti-adrenomedullin (ADM) antibody or an anti-adrenomedullin antibody fragment or anti-ADM non-Ig scaffold is administered 8.4, preferably 8.26 (0.344 days) hours after admission of said patient to ICU.

In a preferred embodiment, the Anti-adrenomedullin (ADM) antibody or an anti-adrenomedullin antibody fragment or anti-ADM non-Ig scaffold is administered within 9, preferably 8.4, preferably 8.26 (0.344 days), preferably 8, preferably 7, preferably 6, preferably 5.76 (0.25 days), preferably 5.75 (0.25 days), preferably 5, preferably 4, preferably 3 hours after the patient has received organ support at the starting point of treatment with said Anti-adrenomedullin (ADM) antibody or an anti-adrenomedullin antibody fragment or anti-ADM non-Ig scaffold.

In a preferred embodiment, the Anti-adrenomedullin (ADM) antibody or an anti-adrenomedullin antibody fragment or anti-ADM non-Ig scaffold is administered 8.4, preferably 8.26 (0.344 days) hours after the patient has received organ support at the starting point of treatment with said Anti-adrenomedullin (ADM) antibody or an anti-adrenomedullin antibody fragment or anti-ADM non-Ig scaffold.

In another specific embodiment of the invention, said shock is selected from the group comprising shock due to hypovolemia, cardiogenic shock, obstructive shock and distributive shock, in particular cardiogenic or septic shock.

In a specific embodiment of the invention, said shock is selected from the group comprising:
in case of cardiogenic shock said patient has suffered an acute coronary syndrome (e.g. acute myocardial infarction) or has heart failure (e.g. acute decompensated heart failure), myocarditis, arrhythmia, cardiomyopathy, valvular heart disease, aortic dissection with acute aortic stenosis, traumatic chordal rupture or massive pulmonary embolism, or
in case of hypovolemic shock said patient may have suffered a hemorrhagic disease including gastrointestinal bleed, trauma, vascular etiologies (e.g. ruptured abdominal aortic aneurysm, tumor eroding into a major blood vessel) and spontaneous bleeding in the setting of anticoagulant use or a non-hemorrhagic disease including vomiting, diarrhea, renal loss, skin losses/insensible losses (e.g. burns, heat stroke) or third-space loss in the setting of pancreatitis, cirrhosis, intestinal obstruction, trauma, or
in case of obstructive shock said patient may have suffered a cardiac tamponade, tension pneumothorax, pulmonary embolism or aortic stenosis, or
in case of distributive shock said patient has septic shock, neurogenic shock, anaphylactic shock or shock due to adrenal crisis.

In a more preferred embodiment, the shock is a septic shock, shock due to Covid-19, shock due to burns or a traumatic shock.

In a most preferred embodiment, the shock was associated with sepsis.

In a further embodiment, the invention relates to an anti-ADM antibody or an anti-ADM antibody fragment or anti-ADM non-Ig scaffold for use in therapy of a patient in shock, in particular septic shock, wherein said patient has been in shock not longer than 10 hours at the starting point of treatment with said an anti-ADM antibody or an anti-ADM antibody fragment or anti-ADM non-Ig scaffold and/or patient who has been admitted to ICU not longer than 10 hours at the starting point of said treatment and/or has not received organ support at all or not longer than 10 hours of organ support at the starting point of treatment with said Anti-adrenomedullin (ADM) antibody or an anti-adrenomedullin antibody fragment or anti-ADM non-Ig scaffold, wherein a sample of bodily fluid taken said patient exhibits a level of bioADM>70 pg/mL, and wherein said bodily fluid is selected from the group comprising whole blood, plasma, serum.

Although a large number of markers were known in the prior art to be connected with shock. nothing in particular suggested DPP3 as a marker of shock. Examples for such biomarkers include MR-proADM, lactate, C-Reactive Protein (CRP) and procalcitonin (PCT) (Ana Maria Navio Serano, 1 Joaquín Valle Alonso, 2, * Gustavo Rene Piñero, 3 Alejandro Rodriguez Camacho, 4 Josefa Soriano Benet, 5 and Manuel Vaquero6 Bull Emerg Trauma. 2019 July; 7(3): 232-239.) and pentraxin 3, heparin-binding protein, soluble triggering receptor, PARK7 and IL-8 cited in a recent review (Charalampos Pierrakos, Dimitrios Velissaris, Max Bisdorff, John C. Marshall & Jean-Louis Vincent Critical Care volume 24, Article number: 287 (2020) Biomarkers of sepsis: time for a reappraisal).

In a preferred embodiment, the bio-ADM is measured from plasma. It is however typical in the technical lifecycle improvement of measurement of analytes that possibilities exist to measure such analytes in other—at least blood-based—matrices, not only plasma. For instance, in case of bio-ADM, another technology has been developed, which uses whole (EDTA-) blood called IB10 Sphingotest® bio-ADM (https://www.nexus-dx.com/wp-content/uploads/2020/07/bio-ADM-IFU-REV-A.pdf). The IB10 Sphingotest® bio-ADM® is a rapid point-of-care (POC) immunoassay for the in vitro quantitative determination of human amidated adrenomedullin peptide (1-52), in the following referred as bioactive adrenomedullin (bio-ADM®), in human EDTA whole blood and plasma.

Dipeptidyl peptidase 3—also known as Dipeptidyl aminopeptidase III, Dipeptidyl arylamidase III, Dipeptidyl peptidase III, Enkephalinase B or red cell angiotensinase; short name: DPP3, DPPIII—is a metallopeptidase that removes dipeptides from physiologically active peptides, such as enkephalins and angiotensins. DPP3 was first identified and its activity measured in extracts of purified bovine anterior pituitary by Ellis & Nuenke 1967. The enzyme, which is listed as EC 3.4.14.4, has a molecular mass of about 83 kDa and is highly conserved in procaryotes and eucaryotes (Prajapati & Chauhan 2011). The amino acid sequence of the human variant is depicted in SEQ ID NO 1. Dipeptidyl peptidase III is a mainly cytosolic peptidase which is ubiquitously expressed. Despite lacking a signal sequence, a few studies reported membranous activity (Lee & Snyder 1982).

DPP3 is a zinc-depending exo-peptidase belonging to the peptidase family M49. It has a broad substrate specificity for oligopeptides from three/four to ten amino acids of various compositions and is also capable of cleaving after proline. DPP3 is known to hydrolyze dipeptides from the N-terminus of its substrates, including angiotensin II, III and IV; Leu- and Met-enkephalin; endomorphin 1 and 2. The metallopeptidase DPP3 has its activity optimum at pH 8.0-9.0 and can be activated by addition of divalent metal ions, such as Co²⁺ and Mg²⁺.

Structural analysis of DPP3 revealed the catalytic motifs HELLGH (hDPP3 450-455) and EECRAE (hDPP3 507-512), as well as following amino acids, that are important for substrate binding and hydrolysis: Glu316, Tyr, 318, Asp366, Asn391, Asn394, His568, Arg572, Arg577, Lys666 and Arg669 (Prajapati & Chauhan 2011; Kumar et al. 2016; numbering refers to the sequence of human DPP3, see SEQ ID NO. 1). Considering all known amino acids or sequence regions that are involved in substrate binding and hydrolysis, the active site of human DPP3 can be defined as the area between amino acids 316 and 669.

The most prominent substrate of DPP3 is angiotensin II (Ang II), the main effector of the renin-angiotensin system (RAS). The RAS is activated in cardiovascular diseases (Dostal et al. 1997. J Mol Cell Cardiol; 29:2893-902; Roks et al. 1997. Heart Vessels. Suppl 12:119-24), sepsis, and septic shock (Correa et al. 2015. Crit Care 2015; 19:98). Ang II, in particular, has been shown to modulate many cardiovascular functions including the control of blood pressure and cardiac remodeling.

Recently, two assays were generated, characterized, and validated to specifically detect DPP3 in human bodily fluids (e.g., blood, plasma, serum): a luminescence immunoassay (LIA) to detect DPP3 protein concentration and an enzyme capture activity assay (ECA) to detect specific DPP3 activity (Rehfeld et al. 2019. JALM 3(6): 943-953). A washing step removes all interfering substances before the actual detection of DPP3 activity is performed. Both methods are highly specific and allow the reproducible detection of DPP3 in blood samples.

Circulating DPP3 levels were shown to be increased in cardiogenic shock patients and were associated with an increased risk of short-term mortality and severe organ dysfunction (Deaniau et al. 2019. Eur J Heart Fail. in press). Moreover, DPP3 measured at inclusion discriminated cardiogenic shock patients who did develop refractory shock vs. non-refractory shock and a DPP3 concentration≥59.1 ng/mL was associated with a greater risk of death (Takagi et al. Eur J Heart Fail. 2020 February; 22(2):279-286).

The invention also relates to an anti-ADM antibody or an anti-ADM antibody fragment or anti-ADM non-Ig scaffold for use in therapy of a patient in shock, in particular septic shock, wherein said patient has been in shock not longer than 10 hours at the starting point of treatment with said an anti-ADM antibody or an anti-ADM antibody fragment or anti-ADM non-Ig scaffold and/or patient who has been admitted to ICU not longer than 9 hours at the starting point of said treatment and/or has not received organ support at all or not longer than 9 hours of organ support at the starting point of treatment with said Anti-adrenomedullin (ADM) antibody or an anti-adrenomedullin antibody fragment or anti-ADM non-Ig scaffold, and wherein a sample of bodily fluid taken said patient exhibits a level of DPP3 below a threshold, and wherein said bodily fluid is selected from the group comprising whole blood, plasma, serum.

In a most preferred embodiment, the said patient is characterized by having a level of DPP3 in a sample of bodily fluid below a threshold, said threshold of DPP3 in a sample of bodily fluid of said patient is between 20 and 120 ng/mL, more preferred between 30 and 80 ng/mL, even more preferred between 40 and 60 ng/mL, most preferred said threshold is 50 ng/mL.

In a specific embodiment of the invention a threshold for the level of DPP3 is the 5fold median concentration, preferably the 4fold median concentration, more preferred the 3fold median concentration, most preferred the 2fold median concentration of a normal healthy population.

The level of DPP3 as the amount of DPP3 protein and/or DPP3 activity in a sample of bodily fluid of said subject may be determined by different methods, e.g. immunoassays, activity assays, mass spectrometric methods etc.

According the present invention, any types of binding assays (immunoassays and analogous assays, which use other types of antigen-specific binders instead of antibodies), and b) DPP3 enzyme activity assays, which are specific for DPP3 by specifically capturing DPP3 from a sample using a specific binder (anti-DPP3 antibody or other type of binder) prior to determination of enzyme activity).

DPP3 activity can be measured by detection of cleavage products of DPP3 specific substrates. Known peptide hormone substrates include Leu-enkephalin, Met-enkephalin, endomorphin 1 and 2, valorphin, β-casomorphin, dynorphin, proctolin, ACTH (Adrenocorticotropic hormone) and MSH (melanocyte-stimulating hormone; Abramíc et al. 2000, Baršun et al. 2007, Dhanda et al. 2008). The cleavage of mentioned peptide hormones as well as other untagged oligopeptides (e.g. Ala-Ala-Ala-Ala, Dhanda et al. 2008) can be monitored by detection of the respective cleavage products. Detection methods include, but are not limited to, HPLC analysis (e.g. Lee & Snyder 1982), mass spectrometry (e.g. Abramić et al. 2000), H1-NMR analysis (e.g. Vandenberg et al. 1985), capillary zone electrophoresis (CE; e.g. Baršun et al. 2007), thin layer chromatography (e.g. Dhanda et al. 2008) or reversed phase chromatography (e.g. Mazocco et al. 2006).

Detection of fluorescence due to hydrolysis of fluorogenic substrates by DPP3 is a standard procedure to monitor DPP3 activity. Those substrates are specific di- or tripeptides (Arg-Arg, Ala-Ala, Ala-Arg, Ala-Phe, Asp-Arg, Gly-Ala, Gly-Arg, Gly-Phe, Leu-Ala, Leu-Gly, Lys-Ala, Phe-Arg, Suc-Ala-Ala-Phe) coupled to a fluorophore. Fluorophores include but are not limited to β-naphtylamide (2-naphtylamide, βNA, 2NA), 4-methoxy-β-naphtylamide (4-methoxy-2-naphtylamide) and 7-amido-4-methylcoumarin (AMC, MCA; Abramić et al. 2000, Ohkubo et al. 1999). Cleavage of these fluorogenic substrates leads to the release of fluorescent β-naphtylamine or 7-amino-4-methylcoumarin respectively. In a liquid phase assay or an ECA substrate and DPP3 are incubated in for example a 96 well plate format and fluorescence is measured using a fluorescence detector (Ellis & Nuenke 1967). Additionally, DPP3 carrying samples can be immobilized and divided on a gel by electrophoresis, gels stained with fluorogenic substrate (e.g. Arg-Arg-βNA) and Fast Garnet GBC and fluorescent protein bands detected by a fluorescence reader (Ohkubo et al. 1999). The same peptides (Arg-Arg, Ala-Ala, Ala-Arg, Ala-Phe, Asp-Arg, Gly-Ala, Gly-Arg, Gly-Phe, Leu-Ala, Leu-Gly, Lys-Ala, Phe-Arg, Suc-Ala-Ala-Phe) can be coupled to chromophores, such as p-nitroanilide diacetate. Detection of color change due to hydrolysis of chromogenic substrates can be used to monitor DPP3 activity.

Another option for the detection of DPP3 activity is a Protease-Glo™ Assay (commercially available at Promega). In this embodiment of said method DPP3 specific di- or tripeptides (Arg-Arg, Ala-Ala, Ala-Arg, Ala-Phe, Asp-Arg, Gly-Ala, Gly-Arg, Gly-Phe, Leu-Ala, Leu-Gly, Lys-Ala, Phe-Arg, Suc-Ala-Ala-Phe) are coupled to aminoluciferin. Upon cleavage by DPP3, aminoluciferin is released and serves as a substrate for a coupled luciferase reaction that emits detectable luminescence.

In a preferred embodiment DPP3 activity is measured by addition of the fluorogenic substrate Arg-Arg-βNA and monitoring fluorescence in real time.

In a specific embodiment of said method for determining active DPP3 in a bodily fluid sample of a subject said capture binder reactive with DPP3 is immobilized on a solid phase.

The test sample is passed over the immobile binder, and DPP3, if present, binds to the binder and is itself immobilized for detection. A substrate may then be added, and the reaction product may be detected to indicate the presence or amount of DPP3 in the test sample. For the purposes of the present description, the term “solid phase” may be used to include any material or vessel in which or on which the assay may be performed and includes, but is not limited to: porous materials, nonporous materials, test tubes, wells, slides, agarose resins (e.g. Sepharose from GE Healthcare Life Sciences), magnetic particals (e.g. Dynabeads™ or Pierce™ magnetic beads from Thermo Fisher Scientific), etc.

In another embodiment of the invention, the level of DPP3 is determined by contacting said sample of bodily fluid with a capture binder that binds specifically to DPP3.

In another preferred embodiment of the invention, said capture binder for determining the level of DPP3 may be selected from the group of antibody, antibody fragment or non-IgG scaffold.

In a specific embodiment of the invention, said capture binder is an antibody.

The amount of DPP3 protein and/or DPP3 activity in a sample of bodily fluid of said subject may be determined for example by one of the following methods:
1. Luminescence immunoassay for the quantification of DPP3 protein concentrations (LIA) (Rehfeld et al., 2019 JALM 3(6): 943-953).

The LIA is a one-step chemiluminescence sandwich immunoassay that uses white high-binding polystyrene microtiter plates as solid phase. These plates are coated with monoclonal anti-DPP3 antibody AK2555 (capture antibody). The tracer anti-DPP3 antibody AK2553 is labeled with MA70-acridinium-NHS-ester and used at a concentration of 20 ng per well. Twenty microliters of samples (e.g. serum, heparin-plasma, citrate-plasma or EDTA-plasma derived from patients' blood) and calibrators are pipetted into coated white microtiter plates. After adding the tracer antibody AK2553, the microtiter plates are incubated for 3 h at room temperature and 600 rpm. Unbound tracer is then removed by 4 washing steps (350 μL per well). Remaining chemiluminescence is measured for is per well by using a microtiter plate luminometer. The concentration of DPP3 is determined with a 6-point calibration curve. Calibrators and samples are preferably run in duplicate.
2. Enzyme capture activity assay for the quantification of DPP3 activity (ECA) (Rehfeld et al., 2019 JALM 3(6): 943-953).

The ECA is a DPP3-specific activity assay that uses black high-binding polystyrene microtiter plates as solid phase. These plates are coated with monoclonal anti-DPP3 antibody AK2555 (capture antibody). Twenty microliters of samples (e.g. serum, heparin-plasma, citrate-plasma, EDTA-plasma, cerebrospinal fluid and urine) and calibrators are pipetted into coated black microtiter plates. After adding assay buffer (200 μL), the microtiter plates are incubated for 2 h at 22° C. and 600 rpm. DPP3 present in the samples is immobilized by binding to the capture antibody. Unbound sample components are removed by 4 washing steps (350 μL per well). The specific activity of immobilized DPP3 is measured by the addition of the fluorogenic substrate, Arg-Arg-β-Naphthylamide (Arg2-PNA), in reaction buffer followed by incubation at 37° C. for 1 h. DPP3 specifically cleaves Arg2-βNA into Arg-Arg dipeptide and fluorescent β-naphthylamine. Fluorescence is measured with a fluorometer using an excitation wavelength of 340 nm and emission is detected at 410 nm. The activity of DPP3 is determined with a 6-point calibration curve. Calibrators and samples are preferably run in duplicates.
3. Liquid-phase assay for the quantification of DPP3 activity (LAA) (modified from Jones et al., Analytical Biochemistry, 1982).

The LAA is a liquid phase assay that uses black non-binding polystyrene microtiter plates to measure DPP3 activity. Twenty microliter of samples (e.g. serum, heparin-plasma, citrate-plasma) and calibrators are pipetted into non-binding black microtiter plates. After addition of fluorogenic substrate, Arg2-βNA, in assay buffer (200 μL), the initial PNA fluorescence (T=0) is measured in a fluorimeter using an excitation wavelength of 340 nm and emission is detected at 410 nm. The plate is then incubated at 37° C. for 1 hour. The final fluorescence of (T=60) is measured. The difference between final and initial fluorescence is calculated. The activity of DPP3 is determined with a 6-point calibration curve. Calibrators and samples are preferably run in duplicates.

In a specific embodiment an assay is used for determining the level of DPP3, wherein the assay sensitivity of said assay is able to quantify the DPP3 of healthy subjects and is <20 ng/ml, preferably <30 ng/ml and more preferably <40 ng/ml.
4. Another immunoassay method for measuring DPP3 from a plasma of whole blood sample is available, IB10 Sphingotest® DPP3 (https://www.nexus-dx.com/wp-content/uploads/2020/11/DPP3-022-00072-IFU-REV-B_8x11.pdf). The IB10 Sphingotest® DPP3 is a rapid point-of-care (POC) immunoassay for the in vitro quantitative determination of Dipeptidyl Peptidase 3 (DPP3) in human EDTA whole blood and plasma. The Nexus IB10 immunochemistry system combines chemistry with microfluidics and centrifugal flow to rapidly prepare a cell free plasma from whole blood that can then be moved through a channel to rehydrate, solubilize and mix with freeze dried immunoconjugates

In a specific embodiment, said binder exhibits a binding affinity to DPP3 of at least 10⁷ M⁻¹, preferred 10⁸ M⁻¹, more preferred affinity is greater than 10⁹ M⁻¹, most preferred greater than 10¹⁰ M⁻¹. A person skilled in the art knows that it may be considered to compensate lower affinity by applying a higher dose of compounds and this measure would not lead out-of-the-scope of the invention.

In another embodiment of the invention, said sample of bodily fluid is selected from the group of whole blood, plasma, and serum.

In a specific embodiment the level of DPP3 is measured with an immunoassay. Immunoassays for the determination of DPP3 are known from the literature. More specifically an immunoassay is used as described e.g. in WO2017/182561. An immunoassay that may be useful for determining the level of DPP3 or fragments thereof of at least 5 amino acids may comprise the steps used in the Examples and referred to in the claims. All thresholds and values have to be seen in correlation to the test and the calibration used according to the Examples. A person skilled in the art may know that the absolute value of a threshold might be influenced by the calibration used. This means that all values and thresholds given herein are to be understood in context of the calibration used.

The threshold is pre-determined by measuring DPP3 concentration and or DPP3 activity in healthy controls and calculating e.g. the according 75-percentile, more preferably the 90-percentile, even more preferably the 95-percentile. The upper boarder of the 75-percentile, more preferably the 90-percentile, even more preferably the 95-percentile, defines the threshold for healthy versus diseased patients. In relation to said percentiles, the threshold that divides between healthy and diseased patients may be between 5 and 25 ng/ml, more preferably between 7 and 20 ng/ml, more preferably between 8 and 18 ng/ml, most preferred between 10 and 15 ng/ml in plasma using a sandwich type anti-DPP3 immunoassay (see example 3). In a DPP3 specific enzyme capture activity assay in plasma, the threshold that divides between healthy and diseased patients may be between 0.5 and 2 nmol βNA min⁻¹ ml⁻¹, more preferably between 0.7 and 1.8 nmol βNA min⁻¹ ml⁻¹, more preferably between 0.8 and 1.5 nmol βNA min⁻¹ ml⁻¹, most preferred between 1.0 and 1.3 nmol βNA min⁻¹ ml⁻¹ (see example 5).

The person skilled in the art knows how to determine thresholds from conducted previous studies. The person skilled in the art knows that a specific threshold value may depend on the cohort used for calculating a pre-determined threshold that can be later-on used in routine. The person skilled in the art knows that a specific threshold value may depend on the calibration used in the assay. The person skilled in the art knows that a specific threshold value may depend on the sensitivity and/or specificity that seems to be acceptable for the practitioner.

The sensitivity and specificity of a diagnostic test depends on more than just the analytical “quality” of the test, they also depend on the definition of what constitutes an abnormal result. In practice, Receiver Operating Characteristic curves (ROC curves), are typically calculated by plotting the value of a variable versus its relative frequency in “normal” (i.e. apparently healthy) and “disease” populations (i.e. patients suffering from an infection). Depending on the particular diagnostic question to be addressed, the reference group must not be necessarily “normals”, but it might be a group of patients suffering from another disease or condition, from which the diseased group of interest shall be differentiated. For any particular marker, a distribution of marker levels for subjects with and without a disease will likely overlap. Under such conditions, a test does not absolutely distinguish normal from disease with 100% accuracy, and the area of overlap indicates where the test cannot distinguish normal from disease. A threshold is selected, above which (or below which, depending on how a marker changes with the disease) the test is considered to be abnormal and below which the test is considered to be normal. The area under the ROC curve is a measure of the probability that the perceived measurement will allow correct identification of a condition. ROC curves can be used even when test results do not necessarily give an accurate number. As long as one can rank results, one can create a ROC curve. For example, results of a test on “disease” samples might be ranked according to degree (e.g. 1=low, 2=normal, and 3=high). This ranking can be correlated to results in the “normal” population, and a ROC curve created. These methods are well known in the art (See, e.g., Hartley et al, 1982). Preferably, a threshold is selected to provide a ROC curve area of greater than about 0.5, more preferably greater than about 0.7. The term “about” in this context refers to +/−5% of a given measurement.

Once the threshold value is determined by using a previous study cohort and taking into consideration all the above-mentioned points the medical practitioner will use the pre-determined threshold for the methods of diagnosing a disease according to the invention and will determine whether the subject has a value above or below said pre-determined threshold value in order to make an appropriate diagnosis.

One embodiment of the present application relates to an anti-ADM antibody or anti-ADM antibody fragment or anti-ADM non-Ig scaffold for use in the treatment of a patient in shock, in particular septic shock, wherein said patient has been in shock not longer than 10 hours at the starting point of treatment with said an anti-ADM antibody or an anti-ADM antibody fragment or anti-ADM non-Ig scaffold and/or patient who has been admitted to ICU not longer than 10 hours at the starting point of said treatment, wherein said patient is additionally characterized by having a level of ADM-NH₂ above a threshold in a sample of body fluid and wherein said body fluid is selected from the group comprising whole blood, plasma, serum.

One embodiment of the present application relates to an anti-ADM antibody or anti-ADM antibody fragment or anti-ADM non-Ig scaffold for use in the treatment of a patient in shock, in particular septic shock, wherein said patient has been in shock not longer than 8.4 hours at the starting point of treatment with said an anti-ADM antibody or an anti-ADM antibody fragment or anti-ADM non-Ig scaffold and/or patient who has been admitted to ICU not longer than 8.4 hours at the starting point of said treatment, wherein said patient is additionally characterized by having a level of ADM-NH₂ above a threshold in a sample of body fluid and wherein said body fluid is selected from the group comprising whole blood, plasma, serum.

One preferred embodiment of the present application said threshold of ADM-NH₂ in the sample of bodily fluid of said patient is between 40 and 100 pg/mL, more preferred between 50 and 90 pg/mL, even more preferred between 60 and 80 pg/mL, most preferred said threshold is 70 pg/mL.

Another embodiment of the present application relates to an anti-ADM antibody or anti-ADM antibody fragment or anti-ADM non-Ig scaffold for use in the treatment or prevention of shock in a patient, wherein the level of ADM-NH₂ is determined by contacting said sample of bodily fluid with a capture binder that binds specifically to ADM-NH₂.

Said antibody or fragment or scaffold binds to N-terminal ADM (SEQ ID NO: 4), as detailed below. Another embodiment of the invention relates to an anti-ADM antibody or an anti-ADM antibody fragment or anti-ADM non-Ig scaffold for use in therapy of a patient in shock, in particular septic shock, wherein said patient has been in shock not longer than 10 hours at the starting point of treatment with said an anti-ADM antibody or an anti-ADM antibody fragment or anti-ADM non-Ig scaffold and/or patient who has been admitted to ICU not longer than 10 hours at the starting point of said treatment, and/or has not received organ support at all or not longer than 10 hours of organ support at the starting point of treatment with said Anti-adrenomedullin (ADM) antibody or an anti-adrenomedullin antibody fragment or anti-ADM non-Ig scaffold, characterized in that said antibody, antibody fragment or non-Ig scaffold bind to the to the midregional part, aa 21-42, of adrenomedullin:

### CTVQKLAHQIYQFTDKDKDNVA (SEQ ID No. 3)

Another embodiment of the invention relates to an anti-ADM antibody or an anti-ADM antibody fragment or anti-ADM non-Ig scaffold for use in therapy of a patient in shock, in particular septic shock, wherein said patient has been in shock not longer than 8.4 hours at the starting point of treatment with said an anti-ADM antibody or an anti-ADM antibody fragment or anti-ADM non-Ig scaffold and/or patient who has been admitted to ICU not longer than 8.4 hours at the starting point of said treatment, and/or has not received organ support at all or not longer than 8.4 hours of organ support at the starting point of treatment with said Anti-adrenomedullin (ADM) antibody or an anti-adrenomedullin antibody fragment or anti-ADM non-Ig scaffold, characterized in that said antibody, antibody fragment or non-Ig scaffold bind to the to the midregional part, aa 21-42, of adrenomedullin:

### CTVQKLAHQIYQFTDKDKDNVA (SEQ ID No. 3)

Another embodiment of the invention relates to an anti-ADM antibody or an anti-ADM antibody fragment or anti-ADM non-Ig scaffold for use in therapy of a patient in shock, in particular septic shock, wherein said patient has been in shock not longer than 10 hours at the starting point of treatment with said an anti-ADM antibody or an anti-ADM antibody fragment or anti-ADM non-Ig scaffold and/or patient who has been admitted to ICU not longer than 10 hours at the starting point of said treatment, and/or has not received organ support at all or not longer than 10 hours of organ support at the starting point of treatment with said Anti-adrenomedullin (ADM) antibody or an anti-adrenomedullin antibody fragment or anti-ADM non-Ig scaffold, and according to any of the preceding embodiments, wherein said antibody or antibody fragment or non-Ig scaffold is monospecific, in particular monoclonal.

Another embodiment of the invention relates to an anti-ADM antibody or an anti-ADM antibody fragment or anti-ADM non-Ig scaffold for use in therapy of a patient in shock, in particular septic shock, wherein said patient has been in shock not longer than 8.4 hours at the starting point of treatment with said an anti-ADM antibody or an anti-ADM antibody fragment or anti-ADM non-Ig scaffold and/or patient who has been admitted to ICU not longer than 8.4 hours at the starting point of said treatment, and/or has not received organ support at all or not longer than 8.4 hours of organ support at the starting point of treatment with said Anti-adrenomedullin (ADM) antibody or an anti-adrenomedullin antibody fragment or anti-ADM non-Ig scaffold, and according to any of the preceding embodiments, wherein said antibody or antibody fragment or non-Ig scaffold is monospecific, in particular monoclonal.

Another embodiment of the invention relates to an anti-ADM antibody or an anti-ADM antibody fragment or anti-ADM non-Ig scaffold for use in therapy of a patient in shock, in particular septic shock, wherein said patient has been in shock not longer than 10 hours at the starting point of treatment with said an anti-ADM antibody or an anti-ADM antibody fragment or anti-ADM non-Ig scaffold and/or patient who has been admitted to ICU not longer than 10 hours at the starting point of said treatment and/or has not received organ support at all or not longer than 10 hours of organ support at the starting point of treatment with said Anti-adrenomedullin (ADM) antibody or an anti-adrenomedullin antibody fragment or anti-ADM non-Ig scaffold, according to any of the preceding embodiments, wherein said antibody or fragment or scaffold exhibits a binding affinity to ADM of at least 10⁻⁷ M by label-free surface plasmon resonance using a Biacore 2000 system. In a more preferred embodiment, the antibody or fragment or scaffold exhibits a binding affinity to ADM exhibits a binding affinity to ADM of between 1×10⁻⁹ to 3×10⁻⁹ by label-free surface plasmon resonance using a Biacore 2000 system. More preferably, the anti-ADM antibody or anti-ADM antibody fragment or anti-ADM non-Ig scaffold is an IgG1 antibody.

Another embodiment of the invention relates to an anti-ADM antibody or an anti-ADM antibody fragment or anti-ADM non-Ig scaffold for use in therapy of a patient in shock, in particular septic shock, wherein said patient has been in shock not longer than 10 hours at the starting point of treatment with said an anti-ADM antibody or an anti-ADM antibody fragment or anti-ADM non-Ig scaffold and/or patient who has been admitted to ICU not longer than 10 hours at the starting point of said treatment, and/or has not received organ support at all or not longer than 10 hours of organ support at the starting point of treatment with said Anti-adrenomedullin (ADM) antibody or an anti-adrenomedullin antibody fragment or anti-ADM non-Ig scaffold, according to any of the preceding embodiments, wherein said antibody or fragment or scaffold is not ADM-binding-Protein-1 (complement factor H).

Another embodiment of the invention relates to an anti-ADM antibody or an anti-ADM antibody fragment or anti-ADM non-Ig scaffold for use in therapy of a patient in shock, in particular septic shock, wherein said patient has been in shock not longer than 8.4 hours at the starting point of treatment with said an anti-ADM antibody or an anti-ADM antibody fragment or anti-ADM non-Ig scaffold and/or patient who has been admitted to ICU not longer than 8.4 hours at the starting point of said treatment, and/or has not received organ support at all or not longer than 8.4 hours of organ support at the starting point of treatment with said Anti-adrenomedullin (ADM) antibody or an anti-adrenomedullin antibody fragment or anti-ADM non-Ig scaffold, according to any of the preceding embodiments, wherein said antibody or fragment or scaffold is not ADM-binding-Protein-1 (complement factor H).

Another embodiment of the invention relates to an anti-ADM antibody or an anti-ADM antibody fragment or anti-ADM non-Ig scaffold for use in therapy of a patient in shock, in particular septic shock, wherein said patient has been in shock not longer than 10 hours at the starting point of treatment with said an anti-ADM antibody or an anti-ADM antibody fragment or anti-ADM non-Ig scaffold and/or patient who has been admitted to ICU not longer than 10 hours at the starting point of said treatment, and/or has not received organ support at all or not longer than 10 hours of organ support at the starting point of treatment with said Anti-adrenomedullin (ADM) antibody or an anti-adrenomedullin antibody fragment or anti-ADM non-Ig scaffold, according to any of the preceding embodiments, wherein said antibody or fragment or scaffold recognizes and binds to the N-terminal end (aa 1) of ADM.

Another embodiment of the invention relates to an anti-ADM antibody or an anti-ADM antibody fragment or anti-ADM non-Ig scaffold for use in therapy of a patient in shock, in particular septic shock, wherein said patient has been in shock not longer than 8.4 hours at the starting point of treatment with said an anti-ADM antibody or an anti-ADM antibody fragment or anti-ADM non-Ig scaffold and/or patient who has been admitted to ICU not longer than 8.4 hours at the starting point of said treatment, and/or has not received organ support at all or not longer than 8.4 hours of organ support at the starting point of treatment with said Anti-adrenomedullin (ADM) antibody or an anti-adrenomedullin antibody fragment or anti-ADM non-Ig scaffold, according to any of the preceding embodiments, wherein said antibody or fragment or scaffold recognizes and binds to the N-terminal end (aa 1) of ADM.

Another embodiment of the invention relates to an anti-ADM antibody or an anti-ADM antibody fragment or anti-ADM non-Ig scaffold for use in therapy of a patient in shock, in particular septic shock, wherein said patient has been in shock not longer than 10 hours at the starting point of treatment with said an anti-ADM antibody or an anti-ADM antibody fragment or anti-ADM non-Ig scaffold and/or patient who has been admitted to ICU not longer than 10 hours at the starting point of said treatment, and or has not received organ support at all or not longer than 10 hours of organ support at the starting point of treatment with said Anti-adrenomedullin (ADM) antibody or an anti-adrenomedullin antibody fragment or anti-ADM non-Ig scaffold, according to any of the preceding embodiments, wherein said antibody or fragment or scaffold is an ADM stabilizing antibody or fragment or scaffold that enhances the half-life (t_{1/2} half retention time) of ADM in serum, blood, plasma at least 10%, preferably at least, 50%, more preferably >50%, most preferably >100%.

Another embodiment of the invention relates to an anti-ADM antibody or an anti-ADM antibody fragment or anti-ADM non-Ig scaffold for use in therapy of a patient in shock, in particular septic shock, wherein said patient has been in shock not longer than 10 hours at the starting point of treatment with said an anti-ADM antibody or an anti-ADM antibody fragment or anti-ADM non-Ig scaffold and/or patient who has been admitted to ICU not longer than 10 hours at the starting point of said treatment, and/or has not received organ support at all or not longer than 10 hours of organ support at the starting point of treatment with said Anti-adrenomedullin (ADM) antibody or an anti-adrenomedullin antibody fragment or anti-ADM non-Ig scaffold, according to any of the preceding embodiments, wherein said antibody or fragment or scaffold blocks the bioactivity of ADM not more than 80%, preferably not more than 50% using hADM 22-52 as a reference antagonist in CHO-K1 cells expressing human recombinant ADM receptor.

Another embodiment of the invention relates a human monoclonal antibody or fragment that binds to ADM or an antibody fragment thereof for use in therapy of a patient in shock, in particular septic shock, wherein said patient has been in shock not longer than 10 hours at the starting point of treatment with said an anti-ADM antibody or an anti-ADM antibody fragment or anti-ADM non-Ig scaffold and/or patient who has been admitted to ICU not longer than 10 hours at the starting point of said treatment, and/or has not received organ support at all or not longer than 10 hours of organ support at the starting point of treatment with said Anti-adrenomedullin (ADM) antibody or an anti-adrenomedullin antibody fragment or anti-ADM non-Ig scaffold, according to any of the preceding embodiments, wherein said antibody or fragment is a human monoclonal antibody or fragment that binds to the N-terminal region (aa 1-21) of ADM (SEQ ID No. 4) or an antibody fragment thereof wherein the heavy chain comprises the sequences:

CDR1:
SEQ ID NO: 5
GYTFSRYW
CDR2:
SEQ ID NO: 6
ILPGSGST
CDR3:
SEQ ID NO: 7
TEGYEYDGFDY

and wherein the light chain comprises the sequences:

CDR1:
SEQ ID NO: 8
QSIVYSNGNTY
CDR2:
RVS
CDR3:
SEQ ID NO: 9
FOGSHIPYT.

In a preferred embodiment of the invention relates a human monoclonal antibody or fragment that binds to ADM or an antibody fragment thereof for use in therapy of a patient in shock, in particular septic shock, wherein said patient has been in shock not longer than 8.4 hours at the starting point of treatment with said an anti-ADM antibody or an anti-ADM antibody fragment or anti-ADM non-Ig scaffold and/or patient who has been admitted to ICU not longer than 8.4 hours at the starting point of said treatment, and/or has not received organ support at all or not longer than 8.4 hours of organ support at the starting point of treatment with said Anti-adrenomedullin (ADM) antibody or an anti-adrenomedullin antibody fragment or anti-ADM non-Ig scaffold according to any of the preceding embodiments, wherein said antibody or fragment is a human monoclonal antibody or fragment that binds to the N-terminal region (aa 1-21) of ADM (SEQ ID No. 4) or an antibody fragment thereof wherein the heavy chain comprises the sequences:

CDR1:
SEQ ID NO: 5
GYTFSRYW
CDR2:
SEQ ID NO: 6
ILPGSGST
CDR3:
SEQ ID NO: 7
TEGYEYDGFDY

and wherein the light chain comprises the sequences:

CDR1:
SEQ ID NO: 8
QSIVYSNGNTY
CDR2:
RVS
CDR3:
SEQ ID NO: 9
FOGSHIPYT.

Another embodiment of the invention relates to a human monoclonal antibody or fragment that binds to ADM or an antibody fragment thereof for use in therapy of a patient in shock, in particular septic shock, wherein said patient has been in shock not longer than 10 hours at the starting point of treatment with said an anti-ADM antibody or an anti-ADM antibody fragment or anti-ADM non-Ig scaffold and/or patient who has been admitted to ICU not longer than 10 hours at the starting point of said treatment, and/or has not received organ support at all or not longer than 10 hours of organ support at the starting point of treatment with said Anti-adrenomedullin (ADM) antibody or an anti-adrenomedullin antibody fragment or anti-ADM non-Ig scaffold, according to any of the preceding embodiments wherein said antibody or fragment comprises a sequence selected from the group comprising as a VH region:

(AM-VH-C)
SEQ ID NO: 10
QVQLQQSGAELMKPGASVKISCKATGYTFSRYWIEWVKQRPGHGLEWIGEILPGSGST
NYNEKFKGKATITADTSSNTAYMQLSSLTSEDSAVYYCTEGYEYDGFDYWGQGTTLTV
SSASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKRVEPKHHHHHH
(AM-VH1)
SEQ ID NO: 11
QVQLVQSGAEVKKPGSSVKVSCKASGYTFSRYWISWVRQAPGQGLEWMGRILPGSGS
TNYAQKFQGRVTITADESTSTAYMELSSLRSEDTAVYYCTEGYEYDGFDYWGQGTTVT
VSSASTKGPSVFPLAPSSKSTSGGTAALGCLVKDYFPEPVTVSWNSGALTSGVHTFPAV
LOSSGLYSLSSVVTVPSSSLGTQTYICNVNHKPSNTKVDKRVEPKHHHHHH
(AM-VH2-E40)
SEQ ID NO: 12
QVQLVQSGAEVKKPGSSVKVSCKASGYTFSRYWIEWVRQAPGQGLEWMGRILPGSGS
TNYAQKFQGRVTITADESTSTAYMELSSLRSEDTAVYYCTEGYEYDGFDYWGQGTTVT
VSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKRVEPKHHHHHH
(AM-VH3-T26-E55)
SEQ ID NO: 13
QVQLVQSGAEVKKPGSSVKVSCKATGYTFSRYWISWVRQAPGQGLEWMGEILPGSGS
TNYAQKFQGRVTITADESTSTAYMELSSLRSEDTAVYYCTEGYEYDGFDYWGQGTTVT
VSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKRVEPKHHHHHH
(AM-VH4-T26-E40-E55)
SEQ ID NO: 14
QVQLVQSGAEVKKPGSSVKVSCKATGYTFSRYWIEWVRQAPGQGLEWMGEILPGSGS
TNYAQKFQGRVTITADESTSTAYMELSSLRSEDTAVYYCTEGYEYDGFDYWGQGTTVT
VSSASTKGPSVFPLAPSSKSTSGGTAALGCLVKDYFPEPVTVSWNSGALTSGVHTFPAV
LOSSGLYSLSSVVTVPSSSLGTQTYICNVNHKPSNTKVDKRVEPKHHHHH

and comprises the following sequence as a VL region:

(AM-VL-C)
SEQ ID NO: 15
DVLLSQTPLSLPVSLGDQATISCRSSQSIVYSNGNTYLEWYLQKPGQSPKLLIYRVSNRF
SGVPDRFSGSGSGTDFTLKISRVEAEDLGVYYCFQGSHIPYTFGGGTKLEIKRTVAAPSV
FIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
(AM-VL1)
SEQ ID NO: 16
DVVMTQSPLSLPVTLGQPASISCRSSQSIVYSNGNTYLNWFQQRPGQSPRRLIYRVSNRD
SGVPDRFSGSGSGTDFTLKISRVEAEDVGVYYCFQGSHIPYTFGQGTKLEIKRTVAAPSV
FIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
(AM-VL2-E40)
SEQ ID NO: 17
DVVMTQSPLSLPVTLGQPASISCRSSQSIVYSNGNTYLEWFQQRPGQSPRRLIYRVSNRD
SGVPDRFSGSGSGTDFTLKISRVEAEDVGVYYCFQGSHIPYTFGQGTKLEIKRTVAAPSV
FIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC

Another embodiment of the invention relates to a human monoclonal antibody or fragment that binds to ADM or an antibody fragment thereof for use in therapy of a patient in shock, in particular septic shock, wherein said patient has been in shock not longer than 8.4 hours at the starting point of treatment with said an anti-ADM antibody or an anti-ADM antibody fragment or anti-ADM non-Ig scaffold and/or patient who has been admitted to ICU not longer than 8.4 hours at the starting point of said treatment, and/or has not received organ support at all or not longer than 8.4 hours of organ support at the starting point of treatment with said Anti-adrenomedullin (ADM) antibody or an anti-adrenomedullin antibody fragment or anti-ADM non-Ig scaffold, according to any of the preceding embodiments wherein said antibody or fragment comprises a sequence selected from the group comprising as a VH region:

(AM-VH-C)
SEQ ID NO: 10
QVQLQQSGAELMKPGASVKISCKATGYTFSRYWIEWVKQRPGHGLEWIGEILPGSGST
NYNEKFKGKATITADTSSNTAYMQLSSLTSEDSAVYYCTEGYEYDGFDYWGQGTTLTV
SSASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKRVEPKHHHHHH
(AM-VH1)
SEQ ID NO: 11
QVQLVQSGAEVKKPGSSVKVSCKASGYTFSRYWISWVRQAPGQGLEWMGRILPGSGS
TNYAQKFQGRVTITADESTSTAYMELSSLRSEDTAVYYCTEGYEYDGFDYWGQGTTVT
VSSASTKGPSVFPLAPSSKSTSGGTAALGCLVKDYFPEPVTVSWNSGALTSGVHTFPAV
LOSSGLYSLSSVVTVPSSSLGTQTYICNVNHKPSNTKVDKRVEPKHHHHHH
(AM-VH2-E40)
SEQ ID NO: 12
TNYAQKFQGRVTITADESTSTAYMELSSLRSEDTAVYYCTEGYEYDGFDYWGQGTTVT
VSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKRVEPKHHHHHH
(AM-VH3-T26-E55)
SEQ ID NO: 13
QVQLVQSGAEVKKPGSSVKVSCKATGYTFSRYWISWVRQAPGQGLEWMGEILPGSGS
TNYAQKFQGRVTITADESTSTAYMELSSLRSEDTAVYYCTEGYEYDGFDYWGQGTTVT
VSSASTKGPSVFPLAPSSKSTSGGTAALGCLVKDYFPEPVTVSWNSGALTSGVHTFPAV
LOSSGLYSLSSVVTVPSSSLGTQTYICNVNHKPSNTKVDKRVEPKHHHHHH
(AM-VH4-T26-E40-E55)
SEQ ID NO: 14
QVQLVQSGAEVKKPGSSVKVSCKATGYTFSRYWIEWVRQAPGQGLEWMGEILPGSGS
TNYAQKFQGRVTITADESTSTAYMELSSLRSEDTAVYYCTEGYEYDGFDYWGQGTTVT
VSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKRVEPKHHHHHH

and comprises the following sequence as a VL region:

(AM-VL-C)
SEQ ID NO: 15
DVLLSQTPLSLPVSLGDQATISCRSSQSIVYSNGNTYLEWYLQKPGQSP
KLLIYRVSNRFSGVPDRFSGSGSGTDFTLKISRVEAEDLGVYYCFQGSH
IPYTFGGGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC
(AM-VL1)
SEQ ID NO: 16
DVVMTQSPLSLPVTLGQPASISCRSSQSIVYSNGNTYLNWFQQRPGQSP
RRLIYRVSNRDSGVPDRFSGSGSGTDFTLKISRVEAEDVGVYYCFQGSH
IPYTFGQGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC
(AM-VL2-E40)
SEQ ID NO: 17
DVVMTQSPLSLPVTLGQPASISCRSSQSIVYSNGNTYLEWFQQRPGQSP
RRLIYRVSNRDSGVPDRFSGSGSGTDFTLKISRVEAEDVGVYYCFQGSH
IPYTFGQGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC

Another embodiment of the invention relates to a human monoclonal antibody or fragment that binds to ADM or an antibody fragment thereof for use in therapy of a patient in shock, in particular septic shock, wherein said patient has been in shock not longer than 10 hours at the starting point of treatment with said an anti-ADM antibody or an anti-ADM antibody fragment or anti-ADM non-Ig scaffold and/or patient who has been admitted to ICU not longer than 10 hours at the starting point of said treatment, and/or has not received organ support at all or not longer than 10 hours of organ support at the starting point of treatment with said Anti-adrenomedullin (ADM) antibody or an anti-adrenomedullin antibody fragment or anti-ADM non-Ig scaffold, according to any of the preceding embodiments, wherein said antibody or fragment comprises the following sequence as a heavy chain:

SEQ ID NO: 22
QVQLVQSGAEVKKPGSSVKVSCKASGYTFSRYWIEWVRQAPGQGLEWIG
EILPGSGSTNYNQKFQGRVTITADTSTSTAYMELSSLRSEDTAVYYCTE
GYEYDGFDYWGQGTTVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNY
KTTPPVLDSDGSFFLYSKLTVDKSRWQQGNVFScSVMHEALHNHYTQKS
LSLSPGK

and comprises the following sequence as a light chain:

SEQ ID NO: 23
DVVLTQSPLSLPVTLGQPASISCRSSQSIVYSNGNTYLEWYLQRPGQSP
RLLIYRVSNRFSGVPDRFSGSGSGTDFTLKISRVEAEDVGVYYCFQGSH
IPYTFGGGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC

In a specific embodiment of the invention the antibody comprises the following sequence as a heavy chain:

SEQ ID NO: 22
QVQLVQSGAEVKKPGSSVKVSCKASGYTFSRYWIEWVRQAPGQGLEWIG
EILPGSGSTNYNQKFQGRVTITADTSTSTAYMELSSLRSEDTAVYYCTE
GYEYDGFDYWGQGTTVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKS
LSLSPGK

or a sequence that is >95% identical to it, preferably >98%, preferably >99% and comprises the following sequence as a light chain:

SEQ ID NO: 23
DVVLTQSPLSLPVTLGQPASISCRSSQSIVYSNGNTYLEWYLQRPGQSP
RLLIYRVSNRFSGVPDRFSGSGSGTDFTLKISRVEAEDVGVYYCFQGSH
IPYTFGGGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC

or a sequence that is >95% identical to it, preferably >98%, preferably >99%.

Another embodiment of the invention relates to a human monoclonal antibody or fragment that binds to ADM or an antibody fragment thereof for use in therapy of a patient in shock, in particular septic shock, wherein said patient has been in shock not longer than 8.4 hours at the starting point of treatment with said an anti-ADM antibody or an anti-ADM antibody fragment or anti-ADM non-Ig scaffold and/or patient who has been admitted to ICU not longer than 8.4 hours at the starting point of said treatment, and/or has not received organ support at all or not longer than 8.4 hours of organ support at the starting point of treatment with said Anti-adrenomedullin (ADM) antibody or an anti-adrenomedullin antibody fragment or anti-ADM non-Ig scaffold, according to any of the preceding embodiments, wherein said antibody or fragment comprises the following sequence as a heavy chain:

SEQ ID NO: 22
QVQLVQSGAEVKKPGSSVKVSCKASGYTFSRYWIEWVRQAPGQGLEWIG
EILPGSGSTNYNQKFQGRVTITADTSTSTAYMELSSLRSEDTAVYYCTE
GYEYDGFDYWGQGTTVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNY
KTTPPVLDSDGSFFLYSKLTVDKSRWQQGNVFScSVMHEALHNHYTQKS
LSLSPGK

and comprises the following sequence as a light chain:

SEQ ID NO: 23
DVVLTQSPLSLPVTLGQPASISCRSSQSIVYSNGNTYLEWYLQRPGQSP
RLLIYRVSNRFSGVPDRFSGSGSGTDFTLKISRVEAEDVGVYYCFQGSH
IPYTFGGGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC

In a specific embodiment of the invention the antibody comprises the following sequence as a heavy chain:

SEQ ID NO: 22
QVQLVQSGAEVKKPGSSVKVSCKASGYTFSRYWIEWVRQAPGQGLEWIG
EILPGSGSTNYNQKFQGRVTITADTSTSTAYMELSSLRSEDTAVYYCTE
GYEYDGFDYWGQGTTVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKS
LSLSPGK

or a sequence that is >95% identical to it, preferably >98%, preferably >99% and comprises the following sequence as a light chain:

SEQ ID NO: 23
DVVLTQSPLSLPVTLGQPASISCRSSQSIVYSNGN
TYLEWYLQRPGQSPRLLIYRVSNRFSGVPDRFSGS
GSGTDFTLKISRVEAEDVGVYYCFQGSHIPYTFGG
GTKLEIKRTVAAPSVFIFPPSDEQLKSGTASVVCL
LNNFYPREAKVQWKVDNALQSGNSQESVTEQDSKD
STYSLSSTLTLSKADYEKHKVYACEVTHQGLSSPV
TKSFNRGEC

or a sequence that is >95% identical to it, preferably >98%, preferably >99%.

To assess the identity between two amino acid sequences, a pairwise alignment is performed. Identity defines the percentage of amino acids with a direct match in the alignment.

In another embodiment, the anti-adrenomedullin (ADM) antibody or anti-ADM antibody fragment or anti-ADM non-Ig scaffold is for use in therapy or prevention of shock in a patient, wherein the anti-adrenomedullin (ADM) antibody or anti-ADM antibody fragment or anti-ADM non-Ig scaffold binds to the N-terminal part (amino acid 1-10) of ADM: YRQSMNNFQG (SEQ ID No. 25).

However, the present invention is not limited to the use of Adrecizumab specifically. There is no reason to doubt that what is true for Adrecizumab will also be true for antibodies sharing main essential features (in particular affinity and epitope specificity) Antibodies that target the same region must be expected to have the same technical effect, provided they have the same affinity and same or very comparable structural features (size, shape, . . . ).

Pharmaceutical composition comprising an antibody according as above outlined.

Another embodiment of the invention relates to an anti-ADM antibody or an anti-ADM antibody fragment or anti-ADM non-Ig scaffold for use in therapy of a patient in shock, in particular septic shock, wherein said patient has been in shock not longer than 10 hours, preferably 8.4 hours (0.35 days) at the starting point of treatment with said an anti-ADM antibody or an anti-ADM antibody fragment or anti-ADM non-Ig scaffold and/or patient who has been admitted to ICU not longer than 10 hours preferably 8.4 hours (0.35 days) at the starting point of said treatment, and/or has not received organ support at all or not longer than 10 hours preferably 8.4 hours (0.35 days) of organ support at the starting point of treatment with said Anti-adrenomedullin (ADM) antibody or an anti-adrenomedullin antibody fragment or anti-ADM non-Ig scaffold, according to any of the preceding embodiments, to be used in combination with known medicaments or other interventions. In particular, the anti-ADM antibody or an anti-ADM antibody fragment or anti-ADM non-Ig scaffold can be used in combination with antimicrobial therapies (antibiotics, anti-mycotics, . . . ), surgical or other mechanical eradication of microbial sources, vasopressors/inotropes, colloids or crystalloids for fluid resuscitation, mechanical ventilation, ECMO (extra corporal membrane oxygenation), extra corporal liver support, renal replacement therapy. In a preferred embodiment, the anti-ADM antibody or an anti-ADM antibody fragment or anti-ADM non-Ig scaffold for use in therapy of a patient in shock, in particular septic shock, is used in combination with a primary medicament. Said primary medicament may be antibiotics in case of infections; vasopressors e.g. catecholamine and/or with fluids administered intravenously. Subject matter of the invention is also an anti-ADM antibody or an anti-adrenomedullin antibody fragment or anti-ADM non-Ig scaffold according to the present invention to be used in combination with TNF-alpha-antibodies.

Another embodiment of the invention relates to a pharmaceutical formulation for use in therapy of a patient in shock, in particular septic shock, wherein said patient has been in shock not longer than 10 hours preferably 8.4 hours (0.35 days) at the starting point of treatment with said an anti-ADM antibody or an anti-ADM antibody fragment or anti-ADM non-Ig scaffold and/or patient who has been admitted to ICU not longer than 10 hours preferably 8.4 hours (0.35 days) at the starting point of said treatment and/or has not received organ support at all or not longer than 10 hours preferably 8.4 hours (0.35 days) of organ support at the starting point of treatment with said Anti-adrenomedullin (ADM) antibody or an anti-adrenomedullin antibody fragment or anti-ADM non-Ig scaffold, comprising an antibody or fragment or scaffold according to any of the preceding embodiments.

Another embodiment of the invention relates to a pharmaceutical formulation for use in for use in therapy of a patient in shock, in particular septic shock, wherein said patient has been in shock not longer than 10 hours preferably 8.4 hours (0.35 days) at the starting point of treatment with said an anti-ADM antibody or an anti-ADM antibody fragment or anti-ADM non-Ig scaffold and/or patient who has been admitted to ICU not longer than 10 hours preferably 8.4 hours (0.35 days) at the starting point of said treatment and/or has not received organ support at all or not longer than 10 hours preferably 8.4 hours (0.35 days) of organ support at the starting point of treatment with said Anti-adrenomedullin (ADM) antibody or an anti-adrenomedullin antibody fragment or anti-ADM non-Ig scaffold, according to the preceding embodiment, wherein said pharmaceutical formulation is a solution, preferably a ready-to-use solution.

Another embodiment of the invention relates to a pharmaceutical formulation for use in for use in therapy of a patient in shock, in particular septic shock, wherein said patient has been in shock not longer than 10 hours preferably 8.4 hours (0.35 days) at the starting point of treatment with said an anti-ADM antibody or an anti-ADM antibody fragment or anti-ADM non-Ig scaffold and/or patient who has been admitted to ICU not longer than 10 hours preferably 8.4 hours (0.35 days) at the starting point of said treatment and/or has not received organ support at all or not longer than 10 hours preferably 8.4 hours (0.35 days) of organ support at the starting point of treatment with said Anti-adrenomedullin (ADM) antibody or an anti-adrenomedullin antibody fragment or anti-ADM non-Ig scaffold, according to the preceding embodiment, wherein said pharmaceutical formulation is in a freeze-dried state.

Another embodiment of the invention relates to a pharmaceutical formulation for use in for use in therapy of a patient in shock, in particular septic shock, wherein said patient has been in shock not longer than 10 hours preferably 8.4 hours (0.35 days) at the starting point of treatment with said an anti-ADM antibody or an anti-ADM antibody fragment or anti-ADM non-Ig scaffold and/or patient who has been admitted to ICU not longer than 10 hours preferably 8.4 hours (0.35 days) at the starting point of said treatment and/or has not received organ support at all or not longer than 10 hours preferably 8.4 hours (0.35 days) of organ support at the starting point of treatment with said Anti-adrenomedullin (ADM) antibody or an anti-adrenomedullin antibody fragment or anti-ADM non-Ig scaffold, according to the preceding embodiment, wherein said pharmaceutical formulation is administered intra-muscular.

Another embodiment of the invention relates to a pharmaceutical formulation for use in for use in therapy of a patient in shock, in particular septic shock, wherein said patient has been in shock not longer than 10 hours preferably 8.4 hours (0.35 days) at the starting point of treatment with said an anti-ADM antibody or an anti-ADM antibody fragment or anti-ADM non-Ig scaffold and/or patient who has been admitted to ICU not longer than 10 hours preferably 8.4 hours (0.35 days) at the starting point of said treatment and/or has not received organ support at all or not longer than 10 hours preferably 8.4 hours (0.35 days) of organ support at the starting point of treatment with said Anti-adrenomedullin (ADM) antibody or an anti-adrenomedullin antibody fragment or anti-ADM non-Ig scaffold, according to the preceding embodiment, wherein said pharmaceutical formulation is administered intra-vascular.

Another embodiment of the invention relates to a pharmaceutical formulation for use in for use in therapy of a patient in shock, in particular septic shock, wherein said patient has been in shock not longer than 10 hours preferably 8.4 hours (0.35 days) at the starting point of treatment with said an anti-ADM antibody or an anti-ADM antibody fragment or anti-ADM non-Ig scaffold and/or patient who has been admitted to ICU not longer than 10 hours preferably 8.4 hours (0.35 days) at the starting point of said treatment and/or has not received organ support at all or not longer than 10 hours preferably 8.4 hours (0.35 days) of organ support at the starting point of treatment with said Anti-adrenomedullin (ADM) antibody or an anti-adrenomedullin antibody fragment or anti-ADM non-Ig scaffold, according to the preceding embodiment, wherein said pharmaceutical formulation is administered via infusion.

Another embodiment of the invention relates to a pharmaceutical formulation for use in for use in therapy of a patient in shock, in particular septic shock, wherein said patient has been in shock not longer than 10 hours preferably 8.4 hours (0.35 days) at the starting point of treatment with said an anti-ADM antibody or an anti-ADM antibody fragment or anti-ADM non-Ig scaffold and/or patient who has been admitted to ICU not longer than 10 hours preferably 8.4 hours (0.35 days) at the starting point of said treatment and/or has not received organ support at all or not longer than 10 hours preferably 8.4 hours (0.35 days) of organ support at the starting point of treatment with said Anti-adrenomedullin (ADM) antibody or an anti-adrenomedullin antibody fragment or anti-ADM non-Ig scaffold, according to the preceding embodiment, wherein said pharmaceutical formulation is to be administered systemically.

Furthermore, in embodiments of the invention an anti-ADM antibody or an anti-ADM antibody fragment or an anti-ADM non-Ig scaffold is monospecific. Monospecific anti-ADM antibody or monospecific anti-ADM antibody fragment or monospecific anti-ADM non-Ig scaffold means that said antibody or antibody fragment or non-Ig scaffold binds to one specific region encompassing at least 5 amino acids within the target ADM. Monospecific anti-ADM antibody or monospecific anti-ADM antibody fragment or monospecific anti-ADM non-Ig scaffold are anti-ADM antibodies or anti-ADM antibody fragments or anti-ADM non-Ig scaffolds that all have affinity for the same antigen.

In a specific and preferred embodiment the present invention provides for a monospecific anti-ADM antibody or monospecific anti-ADM antibody fragment or monospecific anti-ADM non-Ig scaffold, characterized in that said antibody or antibody fragment or non-Ig scaffold binds to one specific region encompassing at least 4 amino acids within the target ADM. In another special embodiment the anti-ADM antibody or the antibody fragment binding to ADM is a monospecific antibody. Monospecific means that said antibody or antibody fragment binds to one specific region encompassing preferably at least 4, or at least 5 amino acids within the target ADM. Monospecific antibodies or fragments are antibodies or fragments that all have affinity for the same antigen. Monoclonal antibodies are monospecific, but monospecific antibodies may also be produced by other means than producing them from a common germ cell.

Another embodiment of the invention relates to an anti-ADM antibody or an anti-ADM antibody fragment or anti-ADM non-Ig scaffold for use in therapy of a patient in shock, in particular septic shock, wherein said Anti-adrenomedullin (ADM) antibody or an anti-adrenomedullin antibody fragment or anti-ADM non-Ig scaffold is administered within 10 hours preferably 8.4 hours (0.35 days) after occurrence of shock in said patient and/or within 10 hours preferably 8.4 hours (0.35 days) after admission of said patient to ICU and/or before the patient has received organ support or within not longer than 10 hours preferably 8.4 hours (0.35 days) of organ support, and wherein said antibody or fragment or scaffold binds to the N-terminal part (aa 1-21) of ADM: YRQSMNNFQGLRSFGCRFGTC (SEQ ID No. 4).

Another embodiment of the invention relates to an anti-ADM antibody or an anti-ADM antibody fragment or anti-ADM non-Ig scaffold for use in therapy of a patient in shock, in particular septic shock, wherein said Anti-adrenomedullin (ADM) antibody or an anti-adrenomedullin antibody fragment or anti-ADM non-Ig scaffold is administered within 10 hours preferably 8.4 hours (0.35 days) after occurrence of shock in said patient and/or within 10 hours preferably 8.4 hours (0.35 days) after admission of said patient to ICU and/or before the patient has received organ support or within not longer than 10 hours preferably 8.4 hours (0.35 days) of organ support, and wherein said antibody or fragment or scaffold binds to the N-terminal part (aa 1-21) of ADM: YRQSMNNFQGLRSFGCRFGTC (SEQ ID No. 4).

The symptoms may be selected from the group of shock that is selected from the group comprising shock due to hypovolemia, cardiogenic shock, obstructive shock and distributive shock.

In another specific embodiment of the invention, said shock is selected from the group comprising shock due to hypovolemia, cardiogenic shock, obstructive shock and distributive shock, in particular cardiogenic or septic shock.

In a specific embodiment of the invention, said shock is selected from the group comprising:
in case of cardiogenic shock said patient has suffered an acute coronary syndrome (e.g. acute myocardial infarction) or has heart failure (e.g. acute decompensated heart failure), myocarditis, arrhythmia, cardiomyopathy, valvular heart disease, aortic dissection with acute aortic stenosis, traumatic chordal rupture or massive pulmonary embolism, or
in case of hypovolemic shock said patient may have suffered a hemorrhagic disease including gastrointestinal bleed, trauma, vascular etiologies (e.g. ruptured abdominal aortic aneurysm, tumor eroding into a major blood vessel) and spontaneous bleeding in the setting of anticoagulant use or a non-hemorrhagic disease including vomiting, diarrhea, renal loss, skin losses/insensible losses (e.g. burns, heat stroke) or third-space loss in the setting of pancreatitis, cirrhosis, intestinal obstruction, trauma, or
in case of obstructive shock said patient may have suffered a cardiac tamponade, tension pneumothorax, pulmonary embolism or aortic stenosis, or
in case of distributive shock said patient has septic shock, neurogenic shock, anaphylactic shock or shock due to adrenal crisis.

In a more preferred embodiment, the shock is a septic shock, shock due to Covid-19, shock due to burns or a traumatic shock. In a most preferred embodiment, the shock was associated with septic shock.

In a preferred embodiment, the invention relates to an anti-adrenomedullin (ADM) antibody or an anti-adrenomedullin antibody fragment or anti-ADM non-Ig scaffold for use in therapy of a patient in shock, in particular septic shock, wherein said Anti-adrenomedullin (ADM) antibody or an anti-adrenomedullin antibody fragment or anti-ADM non-Ig scaffold is administered within 10 hours preferably 8.4 hours (0.35 days) after occurrence of shock in said patient and/or within 10 hours preferably 8.4 hours (0.35 days) after admission of said patient to ICU and/or before the patient has received organ support or within not longer than 10 hours preferably 8.4 hours (0.35 days) of organ support, and wherein said patient is characterized by not having received organ support at all or no longer than 10 hours preferably 8.4 hours (0.35 days).

In a most preferred embodiment, the Anti-adrenomedullin (ADM) antibody or an anti-adrenomedullin antibody fragment or anti-ADM non-Ig scaffold is administered within less than 7, preferably 6, preferably 5, preferably 4, preferably 3 hours after occurrence of shock in said patient. In addition or in another embodiment, said treatment is administered within less than 7, preferably 6, preferably 5, preferably 4, preferably 3 hours after admission of said patient to ICU.

In a further embodiment, the invention relates to an anti-ADM antibody or an anti-ADM antibody fragment or anti-ADM non-Ig scaffold for use in therapy of a patient in shock, in particular septic shock, wherein said Anti-adrenomedullin (ADM) antibody or an anti-adrenomedullin antibody fragment or anti-ADM non-Ig scaffold is administered within 10 hours preferably 8.4 hours (0.35 days) after occurrence of shock in said patient and/or within 10 hours preferably 8.4 hours (0.35 days) after admission of said patient to ICU and/or before the patient has received organ support or within not longer than 10 hours preferably 8.4 hours (0.35 days) of organ support, wherein a sample of bodily fluid taken said patient exhibits a level of bioADM>70 pg/mL, and wherein said bodily fluid is selected from the group comprising whole blood, plasma, serum.

the invention relates to an anti-ADM antibody or an anti-ADM antibody fragment or anti-ADM non-Ig scaffold for use in therapy of a patient in shock, in particular septic shock, wherein said Anti-adrenomedullin (ADM) antibody or an anti-adrenomedullin antibody fragment or anti-ADM non-Ig scaffold is administered within 10 hours preferably 8.4 hours (0.35 days) after occurrence of shock in said patient and/or within 10 hours preferably 8.4 hours (0.35 days) after admission of said patient to ICU and/or before the patient has received organ support or within not longer than 10 hours preferably 8.4 hours (0.35 days) of organ support, wherein a sample of body fluid taken said patient exhibits a level of bioADM>70 pg/mL, and wherein said bodily fluid is selected from the group comprising whole blood, plasma, serum.

The invention also relates to an anti-ADM antibody or an anti-ADM antibody fragment or anti-ADM non-Ig scaffold for use in therapy of a patient in shock, in particular septic shock, wherein said Anti-adrenomedullin (ADM) antibody or an anti-adrenomedullin antibody fragment or anti-ADM non-Ig scaffold is administered within 10 hours preferably 8.4 hours (0.35 days) after occurrence of shock in said patient and/or within 10 hours preferably 8.4 hours (0.35 days) after admission of said patient to ICU and/or before the patient has received organ support or within not longer than 10 hours preferably 8.4 hours (0.35 days) of organ support, wherein a sample of body fluid taken said patient exhibits a level of DPP3 below a threshold, and wherein said bodily fluid is selected from the group comprising whole blood, plasma, serum.

In a most preferred embodiment, the said patient is characterized by having a level of DPP3 in a sample of bodily fluid below a threshold, said threshold of DPP3 in a sample of bodily fluid of said patient is between 20 and 120 ng/mL, more preferred between 30 and 80 ng/mL, even more preferred between 40 and 60 ng/mL, most preferred said threshold is 50 ng/mL.

Said antibody or fragment or scaffold binds to mature ADM, e.g., to ADM of amino acids 1 to 52 (SEQ ID NO: 1), or fragments of mature ADM, e.g., Mid-Regional ADM (MR-ADM) (SEQ ID NO: 3), or to N-terminal ADM (SEQ ID NO: 4), as detailed below.

Therefore, another embodiment of the invention relates to an anti-ADM antibody or an anti-ADM antibody fragment or anti-ADM non-Ig scaffold for use in therapy of a patient in shock, in particular septic shock, wherein said Anti-adrenomedullin (ADM) antibody or an anti-adrenomedullin antibody fragment or anti-ADM non-Ig scaffold is administered within 10 hours preferably 8.4 hours (0.35 days) after occurrence of shock in said patient and/or within 10 hours preferably 8.4 hours (0.35 days) after admission of said patient to ICU and/or before the patient has received organ support or within not longer than 10 hours preferably 8.4 hours (0.35 days) of organ support, wherein said antibody or fragment or scaffold binds to mature ADM, e.g., to ADM of amino acids 1 to 52 (SEQ ID NO: 1), or to fragments thereof as defined above.

Another embodiment of the invention relates to an anti-ADM antibody or an anti-ADM antibody fragment or anti-ADM non-Ig scaffold for use in therapy of a patient in shock, in particular septic shock, wherein said Anti-adrenomedullin (ADM) antibody or an anti-adrenomedullin antibody fragment or anti-ADM non-Ig scaffold is administered within 10 hours preferably 8.4 hours (0.35 days) after occurrence of shock in said patient and/or within 10 hours preferably 8.4 hours (0.35 days) after admission of said patient to ICU and/or before the patient has received organ support or within not longer than 10 hours preferably 8.4 hours (0.35 days) of organ support, according to any of the preceding embodiments, wherein said antibody or fragment or scaffold binds to the N-terminal part (aa 1-21) of ADM:

(SEQ ID NO. 4)
YRQSMNNFQGLRSFGCRFGTC

Another embodiment of the invention relates to an anti-ADM antibody or an anti-ADM antibody fragment or anti-ADM non-Ig scaffold for use in therapy of a patient in shock, in particular septic shock, wherein said Anti-adrenomedullin (ADM) antibody or an anti-adrenomedullin antibody fragment or anti-ADM non-Ig scaffold is administered within 10 hours preferably 8.4 hours (0.35 days) after occurrence of shock in said patient and/or within 10 hours preferably 8.4 hours (0.35 days) after admission of said patient to ICU and/or before the patient has received organ support or within not longer than 10 hours preferably 8.4 hours (0.35 days) of organ support, according to any of the preceding embodiments, characterized in that said antibody, antibody fragment or non-Ig scaffold bind to the to the midregional part, aa 21-42, of adrenomedullin:

(SEQ ID NO. 3)
CTVQKLAHQIYQFTDKDKDNVA

Another embodiment of the invention relates to an anti-ADM antibody or an anti-ADM antibody fragment or anti-ADM non-Ig scaffold for use in therapy of a patient in shock, in particular septic shock, wherein said Anti-adrenomedullin (ADM) antibody or an anti-adrenomedullin antibody fragment or anti-ADM non-Ig scaffold is administered within 10 hours preferably 8.4 hours (0.35 days) after occurrence of shock in said patient and/or within 10 hours preferably 8.4 hours (0.35 days) after admission of said patient to ICU and/or before the patient has received organ support or within not longer than 10 hours preferably 8.4 hours (0.35 days) of organ support, according to any of the preceding embodiments, wherein said antibody or antibody fragment or non-Ig scaffold is monospecific, in particular monoclonal.

Another embodiment of the invention relates to an anti-ADM antibody or an anti-ADM antibody fragment or anti-ADM non-Ig scaffold for use in therapy of a patient in shock, in particular septic shock, wherein said Anti-adrenomedullin (ADM) antibody or an anti-adrenomedullin antibody fragment or anti-ADM non-Ig scaffold is administered within 10 hours preferably 8.4 hours (0.35 days) after occurrence of shock in said patient and/or within 10 hours preferably 8.4 hours (0.35 days) after admission of said patient to ICU and/or before the patient has received organ support or within not longer than 10 hours preferably 8.4 hours (0.35 days) of organ support, according to any of the preceding embodiments, wherein said antibody or fragment or scaffold exhibits a binding affinity to ADM of at least 10⁻⁷ M by label-free surface plasmon resonance using a Biacore 2000 system.

In a more preferred embodiment, the antibody or fragment or scaffold exhibits a binding affinity to ADM exhibits a binding affinity to ADM of between 1×10⁻⁹ to 3×10⁻⁹ by label-free surface plasmon resonance using a Biacore 2000 system. More preferably, the anti-ADM antibody or anti-ADM antibody fragment or anti-ADM non-Ig scaffold is an IgG1 antibody.

Another embodiment of the invention relates to an anti-ADM antibody or an anti-ADM antibody fragment or anti-ADM non-Ig scaffold for use in therapy of a patient in shock, in particular septic shock, wherein said Anti-adrenomedullin (ADM) antibody or an anti-adrenomedullin antibody fragment or anti-ADM non-Ig scaffold is administered within 10 hours preferably 8.4 hours (0.35 days) after occurrence of shock in said patient and/or within 10 hours preferably 8.4 hours (0.35 days) after admission of said patient to ICU and/or before the patient has received organ support or within not longer than 10 hours preferably 8.4 hours (0.35 days) of organ support, according to any of the preceding embodiments, wherein said antibody or fragment or scaffold is not ADM-binding-Protein-1 (complement factor H).

Another embodiment of the invention relates to an anti-ADM antibody or an anti-ADM antibody fragment or anti-ADM non-Ig scaffold for use in therapy of a patient in shock, in particular septic shock, wherein said Anti-adrenomedullin (ADM) antibody or an anti-adrenomedullin antibody fragment or anti-ADM non-Ig scaffold is administered within 10 hours preferably 8.4 hours (0.35 days) after occurrence of shock in said patient and/or within 10 hours preferably 8.4 hours (0.35 days) after admission of said patient to ICU and/or before the patient has received organ support or within not longer than 10 hours preferably 8.4 hours (0.35 days) of organ support, according to any of the preceding embodiments, wherein said antibody or fragment or scaffold recognizes and binds to the N-terminal end (aa 1) of ADM.

Another embodiment of the invention relates to an anti-ADM antibody or an anti-ADM antibody fragment or anti-ADM non-Ig scaffold for use in therapy of a patient in shock, in particular septic shock, wherein said Anti-adrenomedullin (ADM) antibody or an anti-adrenomedullin antibody fragment or anti-ADM non-Ig scaffold is administered within 10 hours preferably 8.4 hours (0.35 days) after occurrence of shock in said patient and/or within 10 hours preferably 8.4 hours (0.35 days) after admission of said patient to ICU and/or before the patient has received organ support or within not longer than 10 hours preferably 8.4 hours (0.35 days) of organ support, according to any of the preceding embodiments, wherein said antibody or fragment or scaffold is an ADM stabilizing antibody or fragment or scaffold that enhances the half-life (t_{1/2} half retention time) of ADM in serum, blood, plasma at least 10%, preferably at least, 50%, more preferably >50%, most preferably >100%.

Another embodiment of the invention relates to an anti-ADM antibody or an anti-ADM antibody fragment or anti-ADM non-Ig scaffold for use in therapy of a patient in shock, in particular septic shock, wherein said Anti-adrenomedullin (ADM) antibody or an anti-adrenomedullin antibody fragment or anti-ADM non-Ig scaffold is administered within 10 hours preferably 8.4 hours (0.35 days) after occurrence of shock in said patient and/or within 10 hours preferably 8.4 hours (0.35 days) after admission of said patient to ICU and/or before the patient has received organ support or within not longer than 10 hours preferably 8.4 hours (0.35 days) of organ support, according to any of the preceding embodiments, wherein said antibody or fragment or scaffold blocks the bioactivity of ADM not more than 80%, preferably not more than 50% using hADM 22-52 as a reference antagonist in CHO-K1 cells expressing human recombinant ADM receptor.

Another embodiment of the invention relates a human monoclonal antibody or fragment that binds to ADM or an antibody fragment thereof for use in therapy of a patient in shock, in particular septic shock, wherein said Anti-adrenomedullin (ADM) antibody or an anti-adrenomedullin antibody fragment or anti-ADM non-Ig scaffold is administered within 10 hours preferably 8.4 hours (0.35 days) after occurrence of shock in said patient and/or within 10 hours preferably 8.4 hours (0.35 days) after admission of said patient to ICU and/or before the patient has received organ support or within not longer than 10 hours preferably 8.4 hours (0.35 days) of organ support, according to any of the preceding embodiments, wherein said antibody or fragment is a human monoclonal antibody or fragment that binds to the N-terminal region (aa 1-21) of ADM (SEQ ID No. 4) or an antibody fragment thereof wherein the heavy chain comprises the sequences:

CDR1:
SEQ ID NO: 5
GYTFSRYW
CDR2:
SEQ ID NO: 6
ILPGSGST
CDR3:
SEQ ID NO: 7
TEGYEYDGFDY

and wherein the light chain comprises the sequences:

CDR1:
SEQ ID NO: 8
QSIVYSNGNTY
CDR2:
RVS
CDR3:
SEQ ID NO: 9
FQGSHIPYT.

Another embodiment of the invention relates to a human monoclonal antibody or fragment that binds to ADM or an antibody fragment thereof for use in therapy of a patient in sepsis and/or shock, in particular septic shock, wherein said Anti-adrenomedullin (ADM) antibody or an anti-adrenomedullin antibody fragment or anti-ADM non-Ig scaffold is administered within 10 hours preferably 8.4 hours (0.35 days) after occurrence of shock in said patient and/or within 10 hours preferably 8.4 hours (0.35 days) after admission of said patient to ICU and/or before the patient has received organ support or within not longer than 10 hours preferably 8.4 hours (0.35 days) of organ support, according to any of the preceding embodiments wherein said antibody or fragment comprises a sequence selected from the group comprising as a VH region:

(AM-VH-C)
SEQ ID NO: 10
QVQLQQSGAELMKPGASVKISCKATGYTFSRYWIE
WVKQRPGHGLEWIGEILPGSGSTNYNEKFKGKATI
TADTSSNTAYMQLSSLTSEDSAVYYCTEGYEYDGF
DYWGQGTTLTVSSASTKGPSVFPLAPSSKSTSGGT
AALGCLVKDYFPEPVTVSWNSGALTSGVHTFPAVL
QSSGLYSLSSVVTVPSSSLGTQTYICNVNHKPSNT
KVDKRVEPKHHHHHH
(AM-VH1)
SEQ ID NO: 11
QVQLVQSGAEVKKPGSSVKVSCKASGYTFSRYWIS
WVRQAPGQGLEWMGRILPGSGSTNYAQKFQGRVTI
TADESTSTAYMELSSLRSEDTAVYYCTEGYEYDGF
DYWGQGTTVTVSSASTKGPSVFPLAPSSKSTSGGT
AALGCLVKDYFPEPVTVSWNSGALTSGVHTFPAVL
QSSGLYSLSSVVTVPSSSLGTQTYICNVNHKPSNT
KVDKRVEPKHHHHHH
(AM-VH2-E40)
SEQ ID NO: 12
QVQLVQSGAEVKKPGSSVKVSCKASGYTFSRYWIE
WVRQAPGQGLEWMGRILPGSGSTNYAQKFQGRVTI
TADESTSTAYMELSSLRSEDTAVYYCTEGYEYDGF
DYWGQGTTVTVSSASTKGPSVFPLAPSSKSTSGGT
AALGCLVKDYFPEPVTVSWNSGALTSGVHTFPAVL
OSSGLYSLSSVVTVPSSSLGTQTYICNVNHKPSNT
KVDKRVEPKHHHHHH
(AM-VH3-T26-E55)
SEQ ID NO: 13
QVQLVQSGAEVKKPGSSVKVSCKATGYTFSRYWIS
WVRQAPGQGLEWMGEILPGSGSTNYAQKFQGRVTI
TADESTSTAYMELSSLRSEDTAVYYCTEGYEYDGF
DYWGQGTTVTVSSASTKGPSVFPLAPSSKSTSGGT
AALGCLVKDYFPEPVTVSWNSGALTSGVHTFPAVL
OSSGLYSLSSVVTVPSSSLGTQTYICNVNHKPSNT
KVDKRVEPKHHHHHH
(AM-VH4-T26-E40-E55)
SEQ ID NO: 14
QVQLVQSGAEVKKPGSSVKVSCKATGYTFSRYWIE
WVRQAPGQGLEWMGEILPGSGSTNYAQKFQGRVTI
TADESTSTAYMELSSLRSEDTAVYYCTEGYEYDGF
DYWGQGTTVTVSSASTKGPSVFPLAPSSKSTSGGT
AALGCLVKDYFPEPVTVSWNSGALTSGVHTFPAVL
OSSGLYSLSSVVTVPSSSLGTQTYICNVNHKPSNT
KVDKRVEPKHHHHHH
and comprises the following
sequence as a VL region:
(AM-VL-C)
SEQ ID NO: 15
DVLLSQTPLSLPVSLGDQATISCRSSQSIVYSNGN
TYLEWYLQKPGQSPKLLIYRVSNRFSGVPDRFSGS
GSGTDFTLKISRVEAEDLGVYYCFQGSHIPYTFGG
GTKLEIKRTVAAPSVFIFPPSDEQLKSGTASVVCL
LNNFYPREAKVQWKVDNALQSGNSQESVTEQDSKD
STYSLSSTLTLSKADYEKHKVYACEVTHQGLSSPV
TKSFNRGEC
(AM-VL1)
SEQ ID NO: 16
DVVMTQSPLSLPVTLGQPASISCRSSQSIVYSNGN
TYLNWFQQRPGQSPRRLIYRVSNRDSGVPDRFSGS
GSGTDFTLKISRVEAEDVGVYYCFQGSHIPYTFGQ
GTKLEIKRTVAAPSVFIFPPSDEQLKSGTASVVCL
LNNFYPREAKVQWKVDNALQSGNSQESVTEQDSKD
STYSLSSTLTLSKADYEKHKVYACEVTHQGLSSPV
TKSFNRGEC
(AM-VL2-E40)
SEQ ID NO: 17
DVVMTQSPLSLPVTLGQPASISCRSSQSIVYSNGN
TYLEWFQQRPGQSPRRLIYRVSNRDSGVPDRFSGS
GSGTDFTLKISRVEAEDVGVYYCFQGSHIPYTFGQ
GTKLEIKRTVAAPSVFIFPPSDEQLKSGTASVVCL
LNNFYPREAKVQWKVDNALQSGNSQESVTEQDSKD
STYSLSSTLTLSKADYEKHKVYACEVTHQGLSSPV
TKSFNRGEC

Another embodiment of the invention relates to a human monoclonal antibody or fragment that binds to ADM or an antibody fragment thereof for use in therapy of a patient in shock, in particular septic shock, wherein said Anti-adrenomedullin (ADM) antibody or an anti-adrenomedullin antibody fragment or anti-ADM non-Ig scaffold is administered within 10 hours preferably 8.4 hours (0.35 days) after occurrence of shock in said patient and/or within 10 hours preferably 8.4 hours (0.35 days) after admission of said patient to ICU and/or before the patient has received organ support or within not longer than 10 hours preferably 8.4 hours (0.35 days) of organ support, according to any of the preceding embodiments, wherein said antibody or fragment comprises the following sequence as a heavy chain: SEQ ID NO: 22

SEQ ID NO: 22
QVQLVQSGAEVKKPGSSVKVSCKASGYTFSRYWIE
WVRQAPGQGLEWIGEILPGSGSTNYNQKFQGRVTI
TADTSTSTAYMELSSLRSEDTAVYYCTEGYEYDGF
DYWGQGTTVTVSSASTKGPSVFPLAPSSKSTSGGT
AALGCLVKDYFPEPVTVSWNSGALTSGVHTFPAVL
QSSGLYSLSSVVTVPSSSLGTQTYICNVNHKPSNT
KVDKKVEPKSCDKTHTCPPCPAPELLGGPSVFLFP
PKPKDTLMISRTPEVTCVVVDVSHEDPEVKFNWYV
DGVEVHNAKTKPREEQYNSTYRVVSVLTVLHQDWL
NGKEYKCKVSNKALPAPIEKTISKAKGQPREPQVY
TLPPSRDELTKNQVSLTCLVKGFYPSDIAVEWESN
GQPENNYKTTPPVLDSDGSFFLYSKLTVDKSRWQQ
GNVFScSVMHEALHNHYTQKSLSLSPGK

and comprises the following sequence as a light chain:

SEQ ID NO: 23
DVVLTQSPLSLPVTLGQPASISCRSSQSIVYSNGN
TYLEWYLQRPGQSPRLLIYRVSNRFSGVPDRFSGS
GSGTDFTLKISRVEAEDVGVYYCFQGSHIPYTFGG
GTKLEIKRTVAAPSVFIFPPSDEQLKSGTASVVCL
LNNFYPREAKVQWKVDNALQSGNSQESVTEQDSKD
STYSLSSTLTLSKADYEKHKVYACEVTHQGLSSPV
TKSFNRGEC

In a specific embodiment of the invention the antibody comprises the following sequence as a heavy chain:

SEQ ID NO: 22
QVQLVQSGAEVKKPGSSVKVSCKASGYTFSRYWIE
WVRQAPGQGLEWIGEILPGSGSTNYNQKFQGRVTI
TADTSTSTAYMELSSLRSEDTAVYYCTEGYEYDGF
DYWGQGTTVTVSSASTKGPSVFPLAPSSKSTSGGT
AALGCLVKDYFPEPVTVSWNSGALTSGVHTFPAVL
LGGPSVFLFPPKPKDTLMISRTPEVTCVVVDVSHE
DPEVKFNWYVDGVEVHNAKTKPREEQYNSTYRVVS
VLTVLHQDWLNGKEYKCKVSNKALPAPIEKTISKA
KGQPREPQVYTLPPSRDELTKNQVSLTCLVKGFYP
SDIAVEWESNGQPENNYKTTPPVLDSDGSFFLYSK
LTVDKSRWQQGNVFSCSVMHEALHNHYTQKSLSLS
PGK

or a sequence that is >95% identical to it, preferably >98%, preferably >99% and comprises the following sequence as a light chain:

SEQ ID NO: 23
DVVLTQSPLSLPVTLGQPASISCRSSQSIVYSNGN
TYLEWYLQRPGQSPRLLIYRVSNRFSGVPDRFSGS
GSGTDFTLKISRVEAEDVGVYYCFQGSHIPYTFGG
GTKLEIKRTVAAPSVFIFPPSDEQLKSGTASVVCL
LNNFYPREAKVQWKVDNALQSGNSQESVTEQDSKD
STYSLSSTLTLSKADYEKHKVYACEVTHQGLSSPV
TKSFNRGEC

or a sequence that is >95% identical to it, preferably >98%, preferably >99%.

To assess the identity between two amino acid sequences, a pairwise alignment is performed. Identity defines the percentage of amino acids with a direct match in the alignment.

In a specific embodiment of the invention the antibody comprises the following sequence as a heavy chain:

SEQ ID NO: 32
QVQLVQSGAEVKKPGSSVKVSCKASGYTFSRYWIE
WVRQAPGQGLEWIGEILPGSGSTNYNQKFQGRVTI
TADTSTSTAYMELSSLRSEDTAVYYCTEGYEYDGF
DYWGQGTTVTVSSASTKGPSVFPLAPSSKSTSGGT
AALGCLVKDYFPEPVTVSWNSGALTSGVHTFPAVL
QSSGLYSLSSVVTVPSSSLGTQTYICNVNHKPSNT
KVDKKVEPKSCDKTHTCPPCPAPELLGGPSVFLFP
PKPKDTLMISRTPEVTCVVVDVSHEDPEVKFNWYV
DGVEVHNAKTKPREEQYNSTYRVVSVLTVLHQDWL
NGKEYKCKVSNKALPAPIEKTISKAKGQPREPQVY
TLPPSRDELTKNQVSLTCLVKGFYPSDIAVEWESN
GQPENNYKTTPPVLDSDGSFFLYSKLTVDKSRWQQ
GNVFSCSVMHEALHNHYTQKSLSLSPGK

or a sequence that comprises CDR-sequences 100% identical to SEQ ID No.: 5, SEQ ID No.: 6 and/or SEQ ID No.:7 and is >95% identical to SEQ ID NO: 22, preferably >98%, preferably >99% and comprises the following sequence as a light chain:

SEQ ID NO: 33
DVVLTQSPLSLPVTLGQPASISCRSSQSIVYSNGN
TYLEWYLQRPGQSPRLLIYRVSNRFSGVPDRFSGS
GSGTDFTLKISRVEAEDVGVYYCFQGSHIPYTFGG
GTKLEIKRTVAAPSVFIFPPSDEQLKSGTASVVCL
LNNFYPREAKVQWKVDNALQSGNSQESVTEQDSKD
STYSLSSTLTLSKADYEKHKVYACEVTHQGLSSPV
TKSFNRGEC

or a sequence that comprises CDR-sequences 100% identical to SEQ ID No.: 8 and/or SEQ ID No.: 9 and is >95% identical to SEQ ID NO: 23, preferably >98%, preferably >99%.

In another embodiment, the anti-adrenomedullin (ADM) antibody or anti-ADM antibody fragment or anti-ADM non-Ig scaffold is for use in therapy or prevention of shock in a patient, wherein the anti-adrenomedullin (ADM) antibody or anti-ADM antibody fragment or anti-ADM non-Ig scaffold binds to the N-terminal part (amino acid 1-10) of ADM: YRQSMNNFQG (SEQ ID No. 25).

Pharmaceutical composition comprising an antibody according as above outlined.

Another embodiment of the invention relates to an anti-ADM antibody or an anti-ADM antibody fragment or anti-ADM non-Ig scaffold for use in therapy of a patient in shock, in particular septic shock, wherein said Anti-adrenomedullin (ADM) antibody or an anti-adrenomedullin antibody fragment or anti-ADM non-Ig scaffold is administered within 10 hours preferably 8.4 hours (0.35 days) after occurrence of shock in said patient and/or within 10 hours preferably 8.4 hours (0.35 days) after admission of said patient to ICU and/or before the patient has received organ support or within not longer than 10 hours preferably 8.4 hours (0.35 days) of organ support, according to any of the preceding embodiments, to be used in combination with known medicaments or other interventions In particular, the anti-ADM antibody or an anti-ADM antibody fragment or anti-ADM non-Ig scaffold can be used in combination with antimicrobial therapies (antibiotics, anti-mycotics, . . . ), surgical or other mechanical eradication of microbial sources, vasopressors/inotropes, colloids or crystalloids for fluid resuscitation, mechanical ventilation, ECMO (extra corporal membrane oxygenation), extra corporal liver support, renal replacement therapy. In a preferred embodiment, the anti-ADM antibody or an anti-ADM antibody fragment or anti-ADM non-Ig scaffold for use in therapy of a patient in shock, in particular septic shock, is used in combination with a primary medicament. Said primary medicament may be antibiotics in case of infections; vasopressors e.g. catecholamine and/or with fluids administered intravenously. Subject matter of the invention is also an anti-ADM antibody or an anti-adrenomedullin antibody fragment or anti-ADM non-Ig scaffold according to the present invention to be used in combination with TNF-alpha-antibodies.

Another embodiment of the invention relates to a pharmaceutical formulation for use in therapy of a patient in shock, in particular septic shock, wherein said Anti-adrenomedullin (ADM) antibody or an anti-adrenomedullin antibody fragment or anti-ADM non-Ig scaffold is administered within 10 hours preferably 8.4 hours (0.35 days) after occurrence of shock in said patient and/or within 10 hours preferably 8.4 hours (0.35 days) after admission of said patient to ICU and/or before the patient has received organ support or within not longer than 10 hours preferably 8.4 hours (0.35 days) of organ support, comprising an antibody or fragment or scaffold according to any of the preceding embodiments.

Another embodiment of the invention relates to a pharmaceutical formulation for use in for use in therapy of a patient in shock, in particular septic shock, wherein said Anti-adrenomedullin (ADM) antibody or an anti-adrenomedullin antibody fragment or anti-ADM non-Ig scaffold is administered within 10 hours preferably 8.4 hours (0.35 days) after occurrence of shock in said patient and/or within 10 hours preferably 8.4 hours (0.35 days) after admission of said patient to ICU and/or before the patient has received organ support or within not longer than 10 hours preferably 8.4 hours (0.35 days) of organ support, according to the preceding embodiment, wherein said pharmaceutical formulation is a solution, preferably a ready-to-use solution.

Another embodiment of the invention relates to a pharmaceutical formulation for use in for use in therapy of a patient in shock, in particular septic shock, wherein said Anti-adrenomedullin (ADM) antibody or an anti-adrenomedullin antibody fragment or anti-ADM non-Ig scaffold is administered within 10 hours preferably 8.4 hours (0.35 days) after occurrence of shock in said patient and/or within 10 hours preferably 8.4 hours (0.35 days) after admission of said patient to ICU and/or before the patient has received organ support or within not longer than 10 hours preferably 8.4 hours (0.35 days) of organ support, according to the preceding embodiment, wherein said pharmaceutical formulation is in a freeze-dried state.

Another embodiment of the invention relates to a pharmaceutical formulation for use in for use in therapy of a patient in shock, in particular septic shock, wherein said Anti-adrenomedullin (ADM) antibody or an anti-adrenomedullin antibody fragment or anti-ADM non-Ig scaffold is administered within 10 hours preferably 8.4 hours (0.35 days) after occurrence of shock in said patient and/or within 10 hours preferably 8.4 hours (0.35 days) after admission of said patient to ICU and/or before the patient has received organ support or within not longer than 10 hours preferably 8.4 hours (0.35 days) of organ support, according to the preceding embodiment, wherein said pharmaceutical formulation is administered intra-muscular.

Another embodiment of the invention relates to a pharmaceutical formulation for use in for use in therapy of a patient in shock, in particular septic shock, wherein said Anti-adrenomedullin (ADM) antibody or an anti-adrenomedullin antibody fragment or anti-ADM non-Ig scaffold is administered within 10 hours preferably 8.4 hours (0.35 days) after occurrence of shock in said patient and/or within 10 hours preferably 8.4 hours (0.35 days) after admission of said patient to ICU and/or before the patient has received organ support or within not longer than 10 hours preferably 8.4 hours (0.35 days) of organ support, according to the preceding embodiment, wherein said pharmaceutical formulation is administered intra-vascular.

Another embodiment of the invention relates to a pharmaceutical formulation for use in for use in therapy of a patient in shock, in particular septic shock, wherein said Anti-adrenomedullin (ADM) antibody or an anti-adrenomedullin antibody fragment or anti-ADM non-Ig scaffold is administered within 10 hours preferably 8.4 hours (0.35 days) after occurrence of shock in said patient and/or within 10 hours preferably 8.4 hours (0.35 days) after admission of said patient to ICU, and/or before the patient has received organ support or within not longer than 10 hours preferably 8.4 hours (0.35 days) of organ support, according to the preceding embodiment, wherein said pharmaceutical formulation is administered via infusion.

Another embodiment of the invention relates to a pharmaceutical formulation for use in for use in therapy of a patient in shock, in particular septic shock, wherein said Anti-adrenomedullin (ADM) antibody or an anti-adrenomedullin antibody fragment or anti-ADM non-Ig scaffold is administered within 10 hours preferably 8.4 hours (0.35 days) after occurrence of shock in said patient, and/or within 10 hours preferably 8.4 hours (0.35 days) after admission of said patient to ICU, and/or before the patient has received organ support or within not longer than 10 hours preferably 8.4 hours (0.35 days) of organ support, wherein said pharmaceutical formulation is to be administered systemically.

An antibody according to the present invention is a protein including one or more polypeptides substantially encoded by immunoglobulin genes that specifically binds an antigen. The recognized immunoglobulin genes include the kappa, lambda, alpha (IgA), gamma (IgG1, IgG2, IgG3, IgG4), delta (IgD), epsilon (IgE) and mu (IgM) constant region genes, as well as the myriad immunoglobulin variable region genes. Full-length immunoglobulin light chains are generally about 25 kDa or 214 amino acids in length. Full-length immunoglobulin heavy chains are generally about 50 kDa or 446 amino acid in length. Light chains are encoded by a variable region gene at the NH₂-terminus (about 110 amino acids in length) and a kappa or lambda constant region gene at the COOH-terminus. Heavy chains are similarly encoded by a variable region gene (about 116 amino acids in length) and one of the other constant region genes.

The basic structural unit of an antibody is generally a tetramer that consists of two identical pairs of immunoglobulin chains, each pair having one light and one heavy chain. In each pair, the light and heavy chain variable regions bind to an antigen, and the constant regions mediate effector functions.

Immunoglobulins also exist in a variety of other forms including, for example, Fv, Fab, and (Fab′)2, as well as bifunctional hybrid antibodies and single chains (e.g., Lanzavecchia et al. 1987. Eur. J. Immunol. 17: 105; Huston et al 1988. PNAS 85:5879-5883; Bird et al. 1988. Science 242:423-426; Hood et al. 1984. Immunology, Benjamin, N.Y., 2nd ed.; Hunkapiller and Hood, 1986. Nature 323: 15-16). An immunoglobulin light or heavy chain variable region includes a framework region interrupted by three hypervariable regions, also called complementarity determining regions (CDR's) (see, Sequences of Proteins of Immunological Interest, E. Kabat et al, U.S. Department of Health and Human Services, 1983). As noted above, the CDRs are primarily responsible for binding to an epitope of an antigen. An immune complex is an antibody, such as a monoclonal antibody, chimeric antibody, humanized antibody or human antibody, or functional antibody fragment, specifically bound to the antigen.

Chimeric antibodies are antibodies whose light and heavy chain genes have been constructed, typically by genetic engineering, from immunoglobulin variable and constant region genes belonging to different species. For example, the variable segments of the genes from a mouse monoclonal antibody can be joined to human constant segments, such as kappa and gamma 1 or gamma 3. In one example, a therapeutic chimeric antibody is thus a hybrid protein composed of the variable or antigen-binding domain from a mouse antibody and the constant or effector domain from a human antibody, although other mammalian species can be used, or the variable region can be produced by molecular techniques. Methods of making chimeric antibodies are well known in the art, e.g., see U.S. Pat. No. 5,807,715. A “humanized” immunoglobulin is an immunoglobulin including a human framework region and one or more CDRs from a non-human (such as a mouse, rat, or synthetic) immunoglobulin. The non-human immunoglobulin providing the CDRs is termed a “donor” and the human immunoglobulin providing the framework is termed an “acceptor.” In one embodiment, all the CDRs are from the donor immunoglobulin in a humanized immunoglobulin. Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDRs. The acceptor framework of a humanized immunoglobulin or antibody may have a limited number of substitutions by amino acids taken from the donor framework. Humanized or other monoclonal antibodies can have additional conservative amino acid substitutions, which have substantially no effect on antigen binding or other immunoglobulin functions. Exemplary conservative substitutions are those such as gly, ala; val, ile, leu; asp, glu; asn, gin; ser, thr; lys, arg; and phe, tyr. Humanized immunoglobulins can be constructed by means of genetic engineering (e.g., see U.S. Pat. No. 5,585,089). A human antibody is an antibody wherein the light and heavy chain genes are of human origin. Human antibodies can be generated using methods known in the art. Human antibodies can be produced by immortalizing a human B cell secreting the antibody of interest. Immortalization can be accomplished, for example, by EBV infection or by fusing a human B cell with a myeloma or hybridoma cell to produce a trioma cell. Human antibodies can also be produced by phage display methods (see, e.g., Dower et al., PCT Publication No. WO 91/17271; McCafferty et al.; PCT Publication No. WO92/001047; and Winter, PCT Publication No. WO 92/20791), or selected from a human combinatorial monoclonal antibody library (see the Morphosys website). Human antibodies can also be prepared by using transgenic animals carrying a human immunoglobulin gene (for example, see PCT Publication No. WO 93/12227; and PCT Publication No. WO 91/10741).

Thus, the anti-ADM antibody may have the formats known in the art. Examples are human antibodies, monoclonal antibodies, humanized antibodies, chimeric antibodies, CDR-grafted antibodies. In a preferred embodiment antibodies according to the present invention are recombinantly produced antibodies as e.g. IgG, a typical full-length immunoglobulin, or antibody fragments containing at least the F-variable domain of heavy and/or light chain as e.g. chemically coupled antibodies (fragment antigen binding) including but not limited to Fab-fragments including Fab minibodies, single chain Fab antibody, monovalent Fab antibody with epitope tags, e.g. Fab-V5Sx2; bivalent Fab (mini-antibody) dimerized with the CH3 domain; bivalent Fab or multivalent Fab, e.g. formed via multirnerization with the aid of a heterologous domain, e.g. via dimerization of dHLX domains, e.g. Fab-dHLX-FSx2; F(ab′)2-fragments, scFv-fragments, multimerized multivalent or/and multispecific scFv-fragments, bivalent and/or bispecific diabodies, BITE® (bispecific T-cell engager), trifunctional antibodies, polyvalent antibodies, e.g. from a different class than G; single-domain antibodies, e.g. nanobodies derived from camelid or fish immunoglobulins and numerous others.

In addition to anti-ADM antibodies other biopolymer scaffolds are well known in the art to complex a target molecule and have been used for the generation of highly target specific biopolymers. Examples are aptamers, spiegelmers, anticalins and conotoxins. For illustration of antibody formats please see FIG. 1 a, 1 b and 1 c in WO 2013/072513.

An antibody fragment according to the present invention is an antigen binding fragment of an antibody according to the present invention.

In a preferred embodiment the ADM antibody format is selected from the group comprising Fv fragment, scFv fragment, Fab fragment, scFab fragment, (Fab)2 fragment and scFv-Fc Fusion protein.

In another preferred embodiment the antibody format is selected from the group comprising scFab fragment, Fab fragment, scFv fragment and bioavailability optimized conjugates thereof, such as PEGylated fragments. One of the most preferred formats is the scFab format.

Non-Ig scaffolds may be protein scaffolds and may be used as antibody mimics as they are capable to bind to ligands or antigens. Non-Ig scaffolds may be selected from the group comprising tetranectin-based non-Ig scaffolds (e.g. described in US 2010/0028995), fibronectin scaffolds (e.g. described in EP 1266 025; lipocalin-based scaffolds ((e.g. described in WO 2011/154420); ubiquitin scaffolds (e.g. described in WO 2011/073214), transferring scaffolds (e.g. described in US 2004/0023334), protein A scaffolds (e.g. described in EP 2231860), ankyrin repeat based scaffolds (e.g. described in WO 2010/060748), microproteins preferably microproteins forming a cystine knot) scaffolds (e.g. described in EP 2314308), Fyn SH3 domain based scaffolds (e.g. described in WO 2011/023685) EGFR-A-domain based scaffolds (e.g. described in WO 2005/040229) and Kunitz domain based scaffolds (e.g. described in EP 1941867).

In one embodiment of the invention antibodies according to the present invention may be produced as follows: A Balb/c mouse was immunized with 100 g ADM-Peptide-BSA-Conjugate at day 0 and 14 (emulsified in 100 μI complete Freund's adjuvant) and 50 g at day 21 and 28 (in 100 μl incomplete Freund's adjuvant). Three days before the fusion experiment was performed, the animal received 50 g of the conjugate dissolved in 100 μI saline, given as one intraperitoneal and one intravenous injection. Splenocytes from the immunized mouse and cells of the myeloma cell line SP2/0 were fused with 1 ml 50% polyethylene glycol for 30 s at 37° C. After washing, the cells were seeded in 96-well cell culture plates. Hybrid clones were selected by growing in HAT medium (RPMI 1640 culture medium supplemented with 20% fetal calf serum and HAT-Supplement). After two weeks the HAT medium is replaced with HT Medium for three passages followed by returning to the normal cell culture medium.

The cell culture supernatants were primary screened for antigen specific IgG antibodies three weeks after fusion. The positive tested microcultures were transferred into 24-well plates for propagation. After retesting, the selected cultures were cloned and recloned using the limiting-dilution technique and the isotypes were determined (see also Lane, R. D. 1985. J. Immunol. Meth. 81: 223-228; Ziegler B. et al. 1996 Horn. Metab. Res. 28: 11-15).

Antibodies may also be produced by means of phage display according to the following procedure: The human naive antibody gene libraries HAL7/8 were used for the isolation of recombinant single chain F-Variable domains (scFv) against ADM peptide. The antibody gene libraries were screened with a panning strategy comprising the use of peptides containing a biotin tag linked via two different spacers to the ADM peptide sequence. A mix of panning rounds using non-specifically bound antigen and streptavidin bound antigen were used to minimize background of non-specific binders. The eluted phages from the third round of panning have been used for the generation of monoclonal scFv expressing E. coli strains. Supernatants from the cultivation of these clonal strains have been directly used for an antigen ELISA testing (see references cited in WO 2013/072513, incorporated herein in their entirety).

Humanization of murine antibodies may be conducted according to the following procedure: For humanization of an antibody of murine origin the antibody sequence is analyzed for the structural interaction of framework regions (FR) with the complementary determining regions (CDR) and the antigen. Based on structural modelling an appropriate FR of human origin is selected and the murine CDR sequences are transplanted into the human FR. Variations in the amino acid sequence of the CDRs or FRs may be introduced to regain structural interactions, which were abolished by the species switch for the FR sequences. This recovery of structural interactions may be achieved by random approach using phage display libraries or via directed approach guided by molecular modeling (Almagro and Fransson 2008. Front Biosci. 13: 1619-33) in a preferred embodiment the ADM antibody format is selected from the group comprising Fv fragment, scFv fragment, Fab fragment, scFab fragment, F(ab)2 fragment and scFv-Fc Fusion protein. In another preferred embodiment the antibody format is selected from the group comprising scFab fragment, Fab fragment, scFv fragment and bioavailability optimized conjugates thereof, such as PEGylated fragments. One of the most preferred formats is scFab format. In another preferred embodiment, the anti-ADM antibody, anti-ADM antibody fragment, or anti-ADM non-Ig scaffold is a full length antibody, antibody fragment, or non-Ig scaffold.

In a preferred embodiment the anti-ADM antibody or an anti-ADM antibody fragment or anti-ADM non-Ig scaffold is directed to and can bind to an epitope of at least 5 amino acids in length contained in ADM.

In another preferred embodiment the anti-ADM antibody or an anti-ADM antibody fragment or anti-ADM non-Ig scaffold is directed to and can bind to an epitope of at least 4 amino acids in length contained in ADM.

In one specific embodiment of the invention the anti-ADM antibody or anti-ADM antibody fragment binding to ADM or anti-ADM non-Ig scaffold binding to ADM is provided for use in therapy of a patient in shock, in particular septic shock, wherein said patient has been in shock not longer than 10 hours preferably 8.4 hours (0.35 days) at the starting point of treatment with said an anti-ADM antibody or an anti-ADM antibody fragment or anti-ADM non-Ig scaffold and/or patient who has been admitted to ICU not longer than 10 preferably 8.4 hours (0.35 days) hours at the starting point of said treatment according to the preceding embodiment, wherein said antibody or fragment or scaffold binds to a region of preferably at least 4, or at least 5 amino acids within the sequence of aa 1-21 of mature human ADM:

(SEQ ID NO .: 4)
YRQSMNNFQGLRSFGCRFGTC.

In one specific embodiment of the invention the anti-ADM antibody or anti-ADM antibody fragment binding to ADM or anti-ADM non-Ig scaffold binding to ADM is provided for use in therapy of a patient in shock, in particular septic shock, wherein said Anti-adrenomedullin (ADM) antibody or an anti-adrenomedullin antibody fragment or anti-ADM non-Ig scaffold is administered within 10 hours preferably 8.4 hours (0.35 days) after occurrence of shock in said patient and/or within 10 hours preferably 8.4 hours (0.35 days) after admission of said patient to ICU, according to the preceding embodiment, wherein said antibody or fragment or scaffold binds to a region of preferably at least 4, or at least 5 amino acids within the sequence of aa 1-21 of mature human ADM:

(SEQ ID NO .: 4)
YRQSMNNFQGLRSFGCRFGTC.

n a preferred embodiment of the present invention said anti-ADM antibody or an anti-ADM antibody fragment or anti-ADM non-Ig scaffold binds to a region of ADM that is located in the N-terminal part (amino acids 1-21) of ADM.

In another preferred embodiment said anti-ADM antibody or an anti-ADM antibody fragment or anti-ADM non-Ig scaffold recognizes and binds to the N-terminal end (aa1) of ADM.

N-terminal end means that the amino acid 1, that is “Y” of SEQ ID No. 1 or 4 is mandatory for antibody binding. Said antibody or fragment or non-Ig scaffold would neither bind N-terminal extended nor N-terminally modified ADM nor N-terminally degraded ADM.

In a preferred embodiment the anti-ADM antibody or an anti-ADM antibody fragment or anti-ADM non-Ig scaffold is directed to and can bind to an epitope of at least 5 amino acids in length contained in ADM, preferably in human ADM.

In a preferred embodiment the anti-ADM antibody or an anti-ADM antibody fragment or anti-ADM non-Ig scaffold is directed to and can bind to an epitope of at least 4 amino acids in length contained in ADM, preferably in human ADM.

In one specific embodiment it is preferred to use an anti-ADM antibody or an anti-ADM antibody fragment or anti-ADM non-Ig scaffold according to the present invention, wherein said anti-ADM antibody or said anti-ADM antibody fragment or anti-ADM non-Ig scaffold is an ADM stabilizing antibody or an ADM stabilizing antibody fragment or an ADM stabilizing non-Ig scaffold that enhances the half-life (t_{1/2}; half retention time) of ADM in serum, blood, plasma at least 10%, preferably at least 50%, more preferably >50%, most preferably >100%. The half-life (half retention time) of ADM may be determined in human plasma in absence and presence of an ADM stabilizing antibody or an ADM stabilizing antibody fragment or an ADM stabilizing non-Ig scaffold, respectively, using an immunoassay for the quantification of ADM.

The following steps may be conducted:
ADM may be diluted in human citrate plasma in absence and presence of an ADM stabilizing antibody or an ADM stabilizing antibody fragment or an ADM stabilizing non-IG scaffold, respectively, and may be incubated at 24° C.;
Aliquots are taken at selected time points (e.g. within 24 hours) and degradation of ADM may be stopped in said aliquots by freezing at −20° C.;
The quantity of ADM may be determined by a hADM immunoassay directly, if the selected assay is not influenced by the stabilizing antibody. Alternatively, the aliquot may be treated with denaturing agents (like HCl) and, after clearing the sample (e.g. by centrifugation) the pH can be neutralized and the ADM-quantified by an ADM immunoassay. Alternatively, non-immunoassay technologies (e.g., RP-HPLC) can be used for ADM-quantification.
The half-life of ADM is calculated for ADM incubated in absence and presence of an ADM stabilizing antibody or an ADM stabilizing antibody fragment or an ADM stabilizing non-IG scaffold, respectively.

The enhancement of half-life is calculated for the stabilized ADM in comparison to ADM that has been incubated in absence of an ADM stabilizing antibody or an ADM stabilizing antibody fragment or an ADM stabilizing non-Ig scaffold.

A two-fold increase of the half-life of ADM is an enhancement of half-life of 100%. Half Life (half retention time) is defined as the period over which the concentration of a specified chemical or drug takes to fall to half baseline concentration in the specified fluid or blood.

In a specific embodiment said anti-ADM antibody, anti-ADM antibody fragment or anti-ADM non-Ig scaffold is a non-neutralizing antibody, fragment or non-Ig scaffold. A neutralizing anti-ADM antibody, anti-ADM antibody fragment or anti-ADM non-Ig scaffold would block the bioactivity of ADM to nearly 100%, to at least more than 90%, preferably to at least more than 95%.

In contrast, a non-neutralizing anti-ADM antibody, or anti-ADM antibody fragment or anti-ADM non-Ig scaffold blocks the bioactivity of ADM less than 100%, preferably to less than 95%, preferably to less than 90%, more preferred to less than 80% and even more preferred to less than 50%. This means that the residual bioactivity of ADM bound to the non-neutralizing anti-ADM antibody, or anti-ADM antibody fragment or anti-ADM non-Ig scaffold would be more than 0%, preferably more than 5%, preferably more than 10%, more preferred more than 20%, more preferred more than 50%. In this context (a) molecule(s), being it an antibody, or an antibody fragment or a non-Ig scaffold with “non-neutralizing anti-ADM activity”, collectively termed here for simplicity as “non-neutralizing” anti-ADM antibody, antibody fragment, or non-Ig scaffold, that e.g. blocks the bioactivity of ADM to less than 80%, is defined as—a molecule or molecules binding to ADM, which upon addition to a culture of an eukaryotic cell line, which expresses functional human recombinant ADM receptor composed of CRLR (calcitonin receptor like receptor) and RAMP3 (receptor-activity modifying protein 3), reduces the amount of cAMP produced by the cell line through the action of parallel added human synthetic ADM peptide, wherein said added human synthetic ADM is added in an amount that in the absence of the non-neutralizing antibody to be analyzed, leads to half-maximal stimulation of cAMP synthesis, wherein the reduction of cAMP by said molecule(s) binding to ADM takes place to an extent, which is not more than 80%, even when the non-neutralizing molecule(s) binding to ADM to be analyzed is added in an amount, which is 10-fold more than the amount, which is needed to obtain the maximal reduction of cAMP synthesis obtainable with the non-neutralizing antibody to be analyzed. The same definition applies to the other ranges; 95%, 90%, 50% etc.

In a specific embodiment according to the present invention an anti-ADM antibody or an anti-ADM antibody fragment or anti-ADM non-Ig scaffold is used, wherein said antibody or antibody fragment or non-Ig scaffold blocks the bioactivity of ADM to less than 80%, preferably less than 50% (of baseline values). This is in the sense of blocking the circulating ADM of not more than 80% or not more than 50%, respectively. It has been understood that said limited blocking of the bioactivity of ADM occurs even at excess concentration of the antibody, antibody fragment or non-Ig scaffold, meaning an excess of the antibody, antibody fragment or non-Ig scaffold in relation to ADM. Said limited blocking is an intrinsic property of the ADM binder itself. This means that said antibody, antibody fragment or non-Ig scaffold have a maximal inhibition of 80% or 50%, respectively. By implication, this means that 20% or 50% residual ADM bioactivity remains present although appropriate amounts or excess amounts of antibody, antibody fragment or non-Ig scaffold are administered, respectively.

In a preferred embodiment said anti-ADM antibody, anti-ADM antibody fragment or anti-ADM non-Ig scaffold would block the bioactivity of ADM at least 5%. By implication, this means residual 95% circulating ADM bioactivity remains present. This is the lower threshold of bioactivity remaining after administration of said anti-ADM antibody, anti-ADM antibody fragment or anti-ADM non-Ig scaffold. The bioactivity is defined as the effect that a substance takes on a living organism or tissue or organ or functional unit in vivo or in vitro (e.g. in an assay) after its interaction. In case of ADM bioactivity this may be the effect of ADM in a human recombinant ADM receptor cAMP functional assay. Thus, according to the present invention bioactivity is defined via an ADM receptor cAMP functional assay. The following steps may be performed in order to determine the bioactivity of ADM in such an assay:
Dose response curves are performed with ADM in said human recombinant ADM receptor cAMP functional assay.
The ADM-concentration of half-maximal cAMP stimulation may be calculated.
At constant half-maximal cAMP-stimulating ADM-concentrations dose response curves (up to 100 μl final concentration) are performed by an ADM stabilizing antibody or an ADM stabilizing antibody fragment or an ADM stabilizing non-Ig scaffold, respectively.

A maximal (at maximal dose) inhibition by said ADM stabilizing antibody of 50% means that said ADM antibody or said ADM antibody fragment or said ADM non-Ig scaffold, respectively, blocks the bioactivity to 50% of baseline values. A maximal inhibition in said ADM bioassay of 80% means that said anti-ADM antibody or said anti-ADM antibody fragment or said anti-ADM non-Ig scaffold, respectively, blocks the bioactivity of ADM to 80%. This is in the sense of blocking the ADM bioactivity to not more than 80%.

In a preferred embodiment a modulating anti-ADM antibody or a modulating anti-ADM antibody fragment or a modulating anti-ADM non-Ig scaffold is used. A “modulating” anti-ADM antibody or a modulating anti-ADM antibody fragment or a modulating anti-ADM non-Ig scaffold is an antibody or an ADM antibody fragment or non-Ig scaffold that enhances the half-life (t half retention time) of ADM in serum, blood, plasma at least 10%, preferably at least, 50%, more preferably >50%, most preferably >100% and blocks the bioactivity of ADM to less than 80%, preferably less than 50% and wherein said anti-ADM antibody, anti-ADM antibody fragment or anti-ADM non-Ig scaffold would block the bioactivity of ADM at least 5%. These values related to half-life and blocking of bioactivity have to be understood in relation to the before-mentioned assays in order to determine these values. This is in the sense of blocking the circulating ADM of not more than 80% or not more than 50%, respectively. This means 20% residual ADM bioactivity remains present, or 50% residual ADM bioactivity remains present, respectively. Such a modulating anti-ADM antibody or a modulating anti-ADM antibody fragment or a modulating anti-ADM non-Ig scaffold offers the advantage that the dosing of the administration is facilitated. The combination of partially blocking or partially reducing ADM bioactivity and increase of the in vivo half-life (increasing the ADM bioactivity) leads to beneficial simplicity of anti-ADM antibody or an anti-ADM antibody fragment or anti-ADM non-Ig scaffold dosing. In a situation of excess endogenous ADM the activity lowering effect is the major impact of the antibody or fragment or scaffold, limiting the (negative) effect of ADM. In case of low or normal endogenous ADM concentrations, the biological effect of anti-ADM antibody or an anti-ADM antibody fragment or anti-ADM non-Ig scaffold is a combination of lowering (by partially blocking) and increase by increasing the ADM half-life. Thus, the non-neutralizing and modulating ADM antibody or ADM antibody fragment or ADM non-Ig scaffold acts like an ADM bioactivity buffer in order to keep the bioactivity of ADM within a certain physiological range.

The anti-ADM antibody or anti-ADM antibody fragment or anti-ADM non-Ig scaffold according to the present invention exhibits an affinity towards human ADM that the affinity constant is greater than 10⁻⁷ M, preferred 10⁻⁸ M, preferred affinity is greater than 10⁻⁹ M, most preferred higher than 10⁻¹⁰ M. A person skilled in the art knows that it may be considered to compensate lower affinity by applying a higher dose of compounds and this measure would not lead out-of-the-scope of the invention. The affinity constants may be determined according to the method as described in Example 1 of WO 2013/072513.

It should be emphasized that the term “ADM binding protein” comprises ADM-binding-protein-1 (complement factor H). However, said ADM binding protein by definition pursuant to the invention is neither a non-neutralizing anti-ADM antibody/antibody fragment/non-Ig scaffold nor a modulating anti-ADM antibody/antibody fragment/non-Ig scaffold. Subject of the present invention is further an anti-ADM antibody or an anti-ADM antibody fragment or anti-ADM non-Ig scaffold for use in therapy of acute disease or acute condition of a patient according to the present invention, wherein said antibody or antibody fragment or non-Ig scaffold may be used in combination with further active ingredients.

Subject of the present invention is further a pharmaceutical formulation comprising an anti-ADM antibody or anti-ADM antibody fragment or anti-ADM non-Ig scaffold according to the present invention. Subject of the present invention is further a pharmaceutical formulation according to the present invention wherein said pharmaceutical formulation is a solution, preferably a ready-to-use solution. In another embodiment subject of the present invention is further a pharmaceutical formulation according to the present invention wherein said pharmaceutical formulation is in a dried state to be reconstituted before use. Said pharmaceutical formulation may be administered intra-muscular. Said pharmaceutical formulation may be administered intra-vascular. Said pharmaceutical formulation may be administered via infusion. In another embodiment subject of the present invention is further a pharmaceutical formulation according to the present invention wherein said pharmaceutical formulation is in a freeze-dried state.

In another more preferred embodiment the present invention provides for a pharmaceutical formulation comprising an anti-ADM antibody or an anti-ADM antibody fragment binding to ADM or anti-ADM non-Ig scaffold binding to ADM for use in therapy of a patient in shock, in particular septic shock, wherein said patient has been in shock not longer than 10 hours preferably 8.4 hours (0.35 days) at the starting point of treatment with said an anti-ADM antibody or an anti-ADM antibody fragment or anti-ADM non-Ig scaffold and/or patient who has been admitted to ICU not longer than 10 hours preferably 8.4 hours (0.35 days) at the starting point of said treatment, and/or patient who has not received organ support at all or not longer than 10 hours preferably 8.4 hours (0.35 days) of organ support at the starting point of treatment with said Anti-adrenomedullin (ADM) antibody or an anti-adrenomedullin antibody fragment or anti-ADM non-Ig scaffold, according to the preceding embodiment, wherein said pharmaceutical formulation is to be administered to a patient in need thereof.

In another embodiment of the present invention the pharmaceutical formulation according to the present invention is to be administered to a patient for therapy a pharmaceutical formulation for use in for use in therapy of a patient in shock, in particular septic shock, wherein said patient has been in shock not longer than 10 hours preferably 8.4 hours (0.35 days) at the starting point of treatment with said an anti-ADM antibody or an anti-ADM antibody fragment or anti-ADM non-Ig scaffold and/or patient who has been admitted to ICU not longer than 10 hours preferably 8.4 hours (0.35 days) at the starting point of said treatment, and/or patient who has not received organ support at all or not longer than 10 hours preferably 8.4 hours (0.35 days) of organ support at the starting point of treatment with said Anti-adrenomedullin (ADM) antibody or an anti-adrenomedullin antibody fragment or anti-ADM non-Ig scaffold, as defined above with the proviso that said patient is in need of such treatment.

In another more preferred embodiment the present invention provides for a pharmaceutical formulation comprising an anti-ADM antibody or an anti-ADM antibody fragment binding to ADM or anti-ADM non-Ig scaffold binding to ADM for use in therapy of a patient in shock, in particular septic shock, wherein said Anti-adrenomedullin (ADM) antibody or an anti-adrenomedullin antibody fragment or anti-ADM non-Ig scaffold is administered within 10 hours preferably 8.4 hours (0.35 days) after occurrence of shock in said patient and/or within 10 hours preferably 8.4 hours (0.35 days) after admission of said patient to ICU, and/or before the patient has received organ support or within not longer than 10 hours preferably 8.4 hours (0.35 days) of organ support, according to the preceding embodiment, wherein said pharmaceutical formulation is to be administered to a patient in need thereof.

In another embodiment of the present invention the pharmaceutical formulation according to the present invention is to be administered to a patient for therapy a pharmaceutical formulation for use in for use in therapy of a patient in shock, in particular septic shock, wherein said Anti-adrenomedullin (ADM) antibody or an anti-adrenomedullin antibody fragment or anti-ADM non-Ig scaffold is administered within 10 hours preferably 8.4 hours (0.35 days) after occurrence of shock in said patient and/or within 10 hours preferably 8.4 hours (0.35 days) after admission of said patient to ICU, and/or before the patient has received organ support or within not longer than 10 hours preferably 8.4 hours (0.35 days) of organ support as defined above with the proviso that said patient is in need of such treatment.

In one embodiment, the ADM antibody or an ADM antibody fragment or ADM non-IG scaffold according to the invention is a non-neutralizing ADM antibody or a non-neutralizing ADM antibody fragment or a non-neutralizing ADM non-Ig scaffold.

As used herein, the antibody format is selected from the group comprising Fv fragment, scFv fragment, Fab fragment, scFab fragment, (Fab)2 fragment and scFv-Fc Fusion protein.

In embodiments of the present invention, the ADM antibody or an ADM antibody fragment or non-Ig-scaffold according to any of the preceding embodiments for use in a treatment of a patient in need thereof wherein said antibody or fragment may be administered in a dose of at least 0.5 mg/Kg body weight, particularly at least 1.0 mg/kg body weight, more particularly, from 1.0 to 20.0 mg/kg body weight, e.g., from 2.0 to 10 mg/kg body weight, from 2.0 to 8.0 mg/kg body weight, or from 2.0 to 5.0 mg/kg body weight.

In preferred embodiments of the present invention, the symptoms of shock that are treated or prevented using any of the ADM antibody or an ADM antibody fragment or non-Ig-scaffold according to any of preceding embodiments are associated with virus infections, wherein said viruses are selected from the group comprising hepadnaviridae, adenoviridae, herpesviridae, influenza viruses, arenaviridae, filoviridae, togaviridae, noroviruses, flaviviridae, retroviridae, measles virus, reoviridae, enteroviridae, picornaviridae, caliciviridae, etc.

In preferred embodiments of the present invention, the symptoms of shock that are treated or prevented using any of the ADM antibody or an ADM antibody fragment or non-Ig-scaffold according to any of preceding embodiments are associated with drug-treatment of primary diseases, such as chemotherapy, therapy with biologics (e.g., antibodies, or fragments thereof), antibiotics, or any medicaments causing any of the above mentioned symptoms of illness.

Further preferred embodiments of the present invention relate to methods of therapy (e.g., treatment, curing, alleviating, improving, amelioration, etc.) or prevention of symptoms as defined in any of the foregoing embodiments comprising administering to a subject in need thereof the ADM antibody or an ADM antibody fragment or non-Ig-scaffold according to any of preceding embodiments. The subject is preferably a human.

Administration of the ADM antibody or an ADM antibody fragment or non-Ig-scaffold can be by any means known in the art, including: orally, intravenously, subcutaneously, intraarterially, intramuscularly, intracardially, intraspinally, intrathoracically, intraperitoneal, intraventricular, sublingually, transdermal, and/or via inhalation. Administration may be systemic, e.g. intravenously, or localized.

As used herein, an “effective dosage” or “effective amount” of drug, compound, or pharmaceutical composition is an amount sufficient to effect beneficial or desired results. For prophylactic use, beneficial or desired results include results such as eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include clinical results such as reducing pain intensity, duration, or frequency of headache attack, and decreasing one or more symptoms resulting from headache (biochemical, histological and/or behavioral), including its complications and intermediate pathological phenotypes presenting during development of the disease, increasing the quality of life of those suffering from the disease, decreasing the dose of other medications required to treat the disease, enhancing effect of another medication, and/or delaying the progression of the disease of patients. An effective dosage can be administered in one or more administrations. For purposes of this invention, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

The following embodiments are subject of the present invention:
1. Anti-adrenomedullin (ADM) antibody or an anti-adrenomedullin antibody fragment or anti-ADM non-Ig scaffold for use in therapy of a patient with shock, in particular septic shock, wherein said patient:
   has suffered from shock, in particular from a septic shock not longer than 10 hours at the starting point of treatment with said Anti-adrenomedullin (ADM) antibody or an anti-adrenomedullin antibody fragment or anti-ADM non-Ig scaffold and/or
   has been admitted to ICU not longer than 10 hours at the starting point of treatment with said Anti-adrenomedullin (ADM) antibody or an anti-adrenomedullin antibody fragment or anti-ADM non-Ig scaffold, and/or
   has not received organ support at all or not longer than 10 hours of organ support at the starting point of treatment with said Anti-adrenomedullin (ADM) antibody or an anti-adrenomedullin antibody fragment or anti-ADM non-Ig scaffold, and
      wherein said antibody or fragment or scaffold binds to the N-terminal part (aa 1-21) of ADM: YRQSMNNFQGLRSFGCRFGTC (SEQ ID No. 4).
2. Anti-Adrenomedullin (ADM) antibody or an anti-ADM antibody fragment binding to ADM or anti-ADM non-Ig scaffold binding to adrenomedullin for use in therapy according to embodiment 1, wherein said patient has suffered from shock, in particular from a septic shock, not longer than 9, preferably 8.4, preferably 8.26 (0.344 days), preferably 8, preferably 7, preferably 6, preferably 5.76 (0.25 days), preferably 5.75 (0.24 days), 5. preferably 4, preferably 3 hours at the starting point of treatment with said Anti-adrenomedullin (ADM) antibody or an anti-adrenomedullin antibody fragment or anti-ADM non-Ig scaffold.
3. Anti-Adrenomedullin (ADM) antibody or an anti-ADM antibody fragment binding to ADM or anti-ADM non-Ig scaffold binding to adrenomedullin for use in therapy according to any of embodiment 1 or 2, wherein said patient has been admitted to ICU not longer than 9, preferably 8.4, preferably 8.26 (0.344 days), preferably 8, preferably 7, preferably 6, preferably 5.76 (0.25 days), preferably 5.75 (0.24 days) preferably 5. preferably 4, preferably 3 hours at the starting point of treatment with said Anti-adrenomedullin (ADM) antibody or an anti-adrenomedullin antibody fragment or anti-ADM non-Ig scaffold.
4. Anti-Adrenomedullin (ADM) antibody or an anti-ADM antibody fragment binding to ADM or anti-ADM non-Ig scaffold binding to adrenomedullin for use in therapy according to any of embodiment 1 to 3, wherein said patient has received organ support not longer than 9, preferably 8.4, preferably 8.26 (0.344 days), preferably 8, preferably 7, preferably 6, preferably 5.76 (0.25 days), preferably 5.75 (0.24 days), preferably 5. preferably 4, preferably 3 hours at the starting point of treatment with said Anti-adrenomedullin (ADM) antibody or an anti-adrenomedullin antibody fragment or anti-ADM non-Ig scaffold
5. Anti-adrenomedullin (ADM) antibody or an anti-adrenomedullin antibody fragment or anti-ADM non-Ig scaffold for use in therapy of a patient suffering from shock, in particular septic shock, wherein said Anti-adrenomedullin (ADM) antibody or an anti-adrenomedullin antibody fragment or anti-ADM non-Ig scaffold is administered
   within 10 hours after occurrence of shock in said patient and/or
   within 10 hours after admission of said patient to ICU, and/or
   before the patient has received organ support or within not longer than 10 hours of organ support, and
   wherein said antibody or fragment or scaffold binds to the N-terminal part (aa 1-21) of ADM: YRQSMNNFQGLRSFGCRFGTC (SEQ ID No. 4).
6. Anti-adrenomedullin (ADM) antibody or an anti-adrenomedullin antibody fragment or anti- to embodiment 5 wherein said Anti-adrenomedullin (ADM) ADM non-Ig scaffold for use in therapy of a patient suffering from shock, in particular septic shock according antibody or an anti-adrenomedullin antibody fragment or anti-ADM non-Ig scaffold is administered within 9, preferably 8.4, preferably 8.26 (0.344 days), preferably 8, preferably 7, preferably 6, preferably 5.76 (0.25 days), preferably 5.75 (0.25 days), preferably 5. preferably 4, preferably 3 hours after occurrence of shock in said patient
7. Anti-adrenomedullin (ADM) antibody or an anti-adrenomedullin antibody fragment or anti-ADM non-Ig scaffold for use in therapy of a patient suffering from shock, in particular septic shock according to embodiment 5 or 6, wherein said Anti-adrenomedullin (ADM) antibody or an anti-adrenomedullin antibody fragment or anti-ADM non-Ig scaffold is administered within 9, preferably 8.4, preferably 8.26 (0.344 days), preferably 8, preferably 7, preferably 6, preferably 5.76 (0.25 days), preferably 5.75 (0.25 days), preferably 5. preferably 4, preferably 3 hours after admission of said patient to ICU.
8. Anti-adrenomedullin (ADM) antibody or an anti-adrenomedullin antibody fragment or anti-ADM non-Ig scaffold for use in therapy of a patient suffering from shock, in particular septic shock according to any of the embodiments 5 to 7, wherein said anti-adrenomedullin (ADM) antibody or an anti-adrenomedullin antibody fragment or anti-ADM non-Ig scaffold is administered within 9, preferably 8.4, preferably 8.26 (0.344 days), preferably 8, preferably 7, preferably 6, preferably 5.76 (0.25 days), preferably 5.75 (0.25 days), preferably 5. preferably 4, preferably 3 hours after the patient has received organ support at the starting point of treatment with said Anti-adrenomedullin (ADM) antibody or an anti-adrenomedullin antibody fragment or anti-ADM non-Ig scaffold.
9. Anti-Adrenomedullin (ADM) antibody or an anti-ADM antibody fragment binding to ADM or anti-ADM non-Ig scaffold binding to adrenomedullin for use in therapy according to any of embodiments 1 to 8, wherein said patient has shock that is selected from the group comprising shock due to hypovolemia, cardiogenic shock, obstructive shock and distributive shock, in particular cardiogenic shock, septic shock, shock due to Covid-19, shock due to burns and traumatic shock.
10. Anti-Adrenomedullin (ADM) antibody or an anti-ADM antibody fragment binding to ADM or anti-ADM non-Ig scaffold binding to adrenomedullin for use in therapy according to embodiment 9, wherein said patient has septic shock.
11. Anti-adrenomedullin (ADM) antibody or an anti-adrenomedullin antibody fragment or anti-ADM non-Ig scaffold for use in therapy of a patient suffering from shock, in particular septic shock, according to any of embodiments 1 to 10, wherein a sample of bodily fluid taken said patient exhibits a level of bioADM>70 pg/mL, and wherein said bodily fluid is selected from the group comprising whole blood, plasma or serum.
12. Anti-adrenomedullin (ADM) antibody or an anti-adrenomedullin antibody fragment or anti-ADM non-Ig scaffold for use in therapy of a patient suffering from shock, in particular septic shock according to any of embodiments 1 to 11, wherein a sample of bodily fluid taken said patient exhibits a level of DPP3<50 ng/mL, and wherein said bodily fluid is selected from the group comprising whole blood, plasma or serum.
13. Anti-adrenomedullin (ADM) antibody or an anti-adrenomedullin antibody fragment or anti-ADM non-Ig scaffold for use in therapy according to any one of embodiments 1 to 12, wherein said antibody or antibody fragment or non-Ig scaffold is monospecific.
14. Anti-adrenomedullin (ADM) antibody or an anti-adrenomedullin antibody fragment or anti-ADM non-Ig scaffold for use in therapy according to any of embodiments 1 to 13, wherein said antibody or fragment or scaffold exhibits a binding affinity to ADM of at least 10-7 M by label-free surface plasmon resonance using a Biacore 2000 system.
15. Anti-adrenomedullin (ADM) antibody or anti-ADM antibody fragment or anti-ADM non-Ig scaffold for use in therapy according to embodiment 14, wherein antibody or fragment or scaffold exhibits a binding affinity to ADM exhibits a binding affinity to ADM of between 1×10−9 to 3×10−9 by label-free surface plasmon resonance using a Biacore 2000 system.
16. Anti-adrenomedullin (ADM) antibody or anti-ADM antibody fragment or anti-ADM non-Ig scaffold for use in therapy or prevention of shock in a patient according to any one of embodiments 13 to 15, wherein the anti-ADM antibody or anti-ADM antibody fragment or anti-ADM non-Ig scaffold is an IgG1 antibody.
17. Anti-adrenomedullin (ADM) antibody or an anti-adrenomedullin antibody fragment or anti-ADM non-Ig scaffold for use in therapy according to any of embodiments 1 to 16, wherein said antibody or fragment or scaffold is not ADM-binding-Protein-1 (complement factor H).
18. Anti-adrenomedullin (ADM) antibody or an anti-adrenomedullin antibody fragment or anti-ADM non-Ig scaffold for use in therapy according to any of the preceding embodiments, wherein said antibody or fragment or scaffold recognizes and binds to the N-terminal end (aa 1) of ADM.
19. Anti-adrenomedullin (ADM) antibody or an anti-adrenomedullin antibody fragment or anti-ADM non-Ig scaffold for use in therapy according to any of the preceding embodiments, wherein said antibody or fragment or scaffold is an ADM stabilizing antibody or fragment or scaffold that enhances the half-life (t½ half retention time) of ADM in serum, blood, plasma at least 10%, preferably at least, 50%, more preferably >50%, most preferably >100%.
20. Anti-adrenomedullin (ADM) antibody or an anti-adrenomedullin antibody fragment or anti-ADM non-Ig scaffold for use in therapy according to any of the preceding embodiments, wherein said antibody or fragment or scaffold blocks the bioactivity of ADM not more than 80%, preferably not more than 50% using hADM 22-52 as a reference antagonist in CHO-K1 cells expressing human recombinant ADM receptor.
21. Anti-adrenomedullin (ADM) antibody or an anti-adrenomedullin antibody fragment or anti-ADM non-Ig scaffold for use in therapy according to any of the preceding items, wherein said subjects undergoes chemotherapy, vasopressors, treatment with biologics, antibiotics, or treatment with anti-viral compounds.
22. Anti-adrenomedullin (ADM) antibody or an anti-adrenomedullin antibody fragment or anti-ADM non-Ig scaffold for use in therapy according to any of the preceding items, wherein said antibody or fragment is a human monoclonal antibody or fragment that binds to ADM or an antibody fragment thereof wherein the heavy chain comprises the sequences:

CDR1:
SEQ ID NO: 5
GYTFSRYW
CDR2:
SEQ ID NO: 6
ILPGSGST
CDR3:
SEQ ID NO: 7
TEGYEYDGFDY

and wherein the light chain comprises the sequences:

| | CDR1: |
|---|---|
| SEQ ID NO: 8 | |
| | QSIVYSNGNTY |
| | CDR2: |
| | RVS |
| | CDR3: |
| SEQ ID NO: 9 | |
| | FQGSHIPYT. |

23. A human monoclonal antibody or fragment that binds to ADM or an antibody fragment thereof for use in therapy according to embodiment 22, wherein said antibody or fragment comprises a sequence selected from the group comprising as a VH region:

(AM-VH-C)
SEQ ID NO: 10
QVQLQQSGAELMKPGASVKISCKATGYTFSRYWIEWVKQRPGHGLEWIGE
ILPGSGSTNYNEKFKGKATITADTSSNTAYMQLSSLTSEDSAVYYCTEGY
EYDGFDYWGQGTTLTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKHHHHHH
(AM-VH1)
SEQ ID NO: 11
QVQLVQSGAEVKKPGSSVKVSCKASGYTFSRYWISWVRQAPGQGLEWMGR
ILPGSGSTNYAQKFQGRVTITADESTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKHHHHHH
(AM-VH2-E40)
SEQ ID NO: 12
QVQLVQSGAEVKKPGSSVKVSCKASGYTFSRYWIEWVRQAPGQGLEWMGR
ILPGSGSTNYAQKFQGRVTITADESTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKHHHHHH
(AM-VH3-T26-E55)
SEQ ID NO: 13
QVQLVQSGAEVKKPGSSVKVSCKATGYTFSRYWISWVRQAPGQGLEWMGE
ILPGSGSTNYAQKFQGRVTITADESTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKHHHHHH
(AM-VH4-T26-E40-E55)
SEQ ID NO: 14
QVQLVQSGAEVKKPGSSVKVSCKATGYTFSRYWIEWVRQAPGQGLEWMGE
ILPGSGSTNYAQKFQGRVTITADESTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLOSSGLYSLSSVVTVPSSSLGTQTYI
CNVNHKPSNTKVDKRVEPKHHHHHH

and comprises a sequence selected from the group comprising the following sequence as a VL region:

(AM-VL-C)
SEQ ID NO: 15
DVLLSQTPLSLPVSLGDQATISCRSSQSIVYSNGNTYLEWYLQKPGQSPK
LLIYRVSNRFSGVPDRFSGSGSGTDFTLKISRVEAEDLGVYYCFQGSHIP
YTFGGGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
(AM-VL1)
SEQ ID NO: 16
DVVMTQSPLSLPVTLGQPASISCRSSQSIVYSNGNTYLNWFQQRPGQSPR
RLIYRVSNRDSGVPDRFSGSGSGTDFTLKISRVEAEDVGVYYCFQGSHIP
YTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
(AM-VL2-E40)
SEQ ID NO: 17
DVVMTQSPLSLPVTLGQPASISCRSSQSIVYSNGNTYLEWFQQRPGQSPR
RLIYRVSNRDSGVPDRFSGSGSGTDFTLKISRVEAEDVGVYYCFQGSHIP
YTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC.

24. A human monoclonal antibody or fragment that binds to ADM or an antibody fragment thereof for use in therapy according to embodiment 22 or 23, wherein said antibody or fragment comprises the following sequence as a heavy chain:

SEQ ID NO: 22
QVQLVQSGAEVKKPGSSVKVSCKASGYTFSRYWIEWVRQAPGQGLEWIGE
ILPGSGSTNYNQKFQGRVTITADTSTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK

or a sequence that is >95% identical to it,
and comprises the following sequence as a light chain:

SEQ ID NO: 23
DVVLTQSPLSLPVTLGQPASISCRSSQSIVYSNGNTYLEWYLQRPGQSPR
LLIYRVSNRFSGVPDRFSGSGSGTDFTLKISRVEAEDVGVYYCFQGSHIP
YTFGGGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

or a sequence that is >95% identical to it.
25. Anti-adrenomedullin (ADM) antibody or anti-ADM antibody fragment or anti-ADM non-Ig scaffold for use in therapy or prevention of shock in a patient according to embodiments 1-24, wherein the anti-adrenomedullin (ADM) antibody or anti-ADM antibody fragment or anti-ADM non-Ig scaffold binds to the N-terminal part (amino acid 1-10) of ADM: YRQSMNNFQG (SEQ ID No. 25).
26. Pharmaceutical formulation for use in therapy or prevention of symptoms of illness or for the use in therapy according to any of embodiments 1 to 25.

### DESCRIPTION OF THE FIGURES

FIG. 1 shows the mortality rate over time in patients under Adrecizumab treatment compared to placebo when the treatment was administered between 0 and 10 hours after shock was diagnosed (FIG. 1A) and between 10 and 12.2 hours post shock (FIG. 1B). HR [95% CI] and log-rank p-values were as follows: Group 0-10 h after shock diagnosis: HR=0.439 [0.174-1.11], log rank p=0.072; Group 10-12.2 h after shock diagnosis: HR=0.711 [0.326-1.55], log rank p=0.387.

FIG. 2 shows the changes of the SOFA Score when Adrecizumab treatment was administered within within 10 h after shock diagnosis (FIG. 2A) compared to treatment administered more than 10 h post shock diagnosis (FIG. 2B). The SOFA score was determined immediately prior to dosing and on the following days. The difference between the SOFA score on each time point to the pre-dose/placebo SOFA score was calculated for each patient, and the means from the resulting values are represented in the graph. In the analysis only patients with recorded values for all SOFA components were included. A “SOFA+” score was defined by handling the missing data as follows: For patients discharged, the SOFA was set to 0 at the respective time points, for patients who died the SOFA was set to 24.

FIG. 3 shows the 28-day mortality rate over time in patients under Adrecizumab treatment compared to placebo when the treatment was administered between 0 and 0.344 days (8.3 hours) after ICU admission (FIG. 3A) and between 0.344 days and 29 days after ICU admission (FIG. 3B). HR [95% CI] and log-rank p-values were as follows: Group 0-0.34 days ICU stay till treatment/28-day mortality: HR=0.263 [0.077-0.898], log rank p=0.022; Group 0.34-29 days ICU stay till treatment/28-day mortality: HR=0.734 [0.358-1.5], log rank p=0.399.

FIG. 4 shows the 90-day mortality rate over time in patients under Adrecizumab treatment compared to placebo when the treatment was administered between 0 and 0.344 days (8.3 hours) after ICU admission (FIG. 4A) and between 0.344 days and 29 days after ICU admission (FIG. 4B). Group 0-0.34 days ICU stay till treatment/90-day mortality: HR=0.364 [0.137-0.97], log rank p=0.035; Group 0.34-29 days ICU stay till treatment/90-day mortality: HR=0.826 [0.469-1.45], log rank p=0.51.

FIG. 5 shows the changes of the SOFA Score when Adrecizumab treatment was administered within 0.344 days after ICU admission (FIG. 5A) compared to treatment administered more than 0.344 days post ICU admission (FIG. 5B). The SOFA score was determined immediately prior to dosing and on the following days. The difference between the SOFA score on each time point to the pre-dose/placebo SOFA score was calculated for each patient, and the means from the resulting values are represented in the graph. In the analysis only patients with recorded values for all SOFA components were included. A “SOFA+” score was defined by handling the missing data as follows: For patients discharged, the SOFA was set to 0 at the respective time points, for patients who died the SOFA was set to 24.

FIG. 6 shows the mean fluid balance on day 7 after start of treatment, when Adrecizumab treatment was administered 0.344 days after ICU admission (FIG. 6A) compared to treatment administered more than 0.344 days post ICU admission (FIG. 6B). The fluid balance (fluid input minus fluid output) was recorded for each 24 h period on the ICU. The mean fluid balance up to 7 days after Adrecizumab/placebo infusion was calculated per patient. Days with missing fluid balance, e.g. due to discharge from the ICU or death, were not considered. Medians+IQR are shown as bar plots.

FIG. 7 shows the mean fluid balance on day 7 after start of treatment, when Adrecizumab treatment was administered within 10 hours after shock diagnosis (FIG. 7A) compared to treatment administered more than 10 hours after shock diagnosis (FIG. 7B). The mean fluid balance up to 7 days after Adrecizumab/placebo infusion was calculated per patient. Days with missing fluid balance, e.g. due to discharge from the ICU or death, were not considered. Medians+IQR are shown as bar plots.

FIG. 8 shows the DPP3 concentrations from septic shock patients on the day of ICU admission and on the following day.

FIG. 9 shows the individual trajectories of DPP3 concentrations from septic shock patients. The x-axis denotes days after ICU admission (with day 1 being the day of admission).

### EXAMPLES

Generation of Antibodies and Determination of their Affinity Constants

Several human and murine antibodies were produced and their affinity constants were determined (see Table 1).

Peptides/Conjugates for Immunization:

Peptides for immunization were synthesized, see Table 1, (JPT Technologies, Berlin, Germany) with an additional N-terminal Cysteine (if no Cysteine is present within the selected ADM-sequence) residue for conjugation of the peptides to Bovine Serum Albumin (BSA). The peptides were covalently linked to BSA by using Sulfolink-coupling gel (Perbio Science, Bonn, Germany). The coupling procedure was performed according to the manual of Perbio.

The murine antibodies were generated according to the following method:

A Balb/c mouse was immunized with 100 μg Peptide-BSA-Conjugate at day 0 and 14 (emulsified in 100 μl complete Freund's adjuvant) and 50 μg at day 21 and 28 (in 100 μl incomplete Freund's adjuvant).

Three days before the fusion experiment was performed, the animal received 50 μg of the conjugate dissolved in 100 μl saline, given as one intraperitoneal and one intra-venous injection.

Splenocytes from the immunized mouse and cells of the myeloma cell line SP2/0 were fused with 1 ml 50% polyethylene glycol for 30 s at 37° C. After washing, the cells were seeded in 96-well cell culture plates. Hybrid clones were selected by growing in HAT medium (RPMI 1640 culture medium supplemented with 20% fetal calf serum and HAT-Supplement). After two weeks the HAT medium is replaced with HT Medium for three passages followed by returning to the normal cell culture medium.

The cell culture supernatants were primary screened for antigen specific IgG antibodies three weeks after fusion. The positive tested microcultures were transferred into 24-well plates for propagation. After retesting, the selected cultures were cloned and recloned using the limiting-dilution technique and the isotypes were determined (see also Lane, R. D. 1985. J. Immunol. Meth. 81: 223-228; Ziegler et al. 1996 Horm. Metab. Res. 28: 11-15).

Mouse Monoclonal Antibody Production:

Antibodies were produced via standard antibody production methods (Marx et al, 1997. Monoclonal Antibody Production, ATLA 25, 121) and purified via Protein A. The antibody purities were >95% based on SDS gel electrophoresis analysis.

Human Antibodies:

Human Antibodies were produced by means of phage display according to the following procedure: The human naive antibody gene libraries HAL7/8 were used for the isolation of recombinant single chain F-Variable domains (scFv) against ADM peptide. The antibody gene libraries were screened with a panning strategy comprising the use of peptides containing a biotin tag linked via two different spacers to the ADM peptide sequence. A mix of panning rounds using non-specifically bound antigen and streptavidin bound antigen were used to minimize background of non-specific binders. The eluted phages from the third round of panning have been used for the generation of monoclonal scFv expressing E. coli strains. Supernatant from the cultivation of these clonal strains has been directly used for an antigen ELISA testing (see also Hust et al. 2011. Journal of Biotechnology 152, 159-170; Schatte et al. 2009. PLoS One 4, e6625).

Positive clones have been selected based on positive ELISA signal for antigen and negative for streptavidin coated micro titer plates. For further characterizations the scFv open reading frame has been cloned into the expression plasmid pOPE107 (Hust et al. 2011. Journal of Biotechnology 152, 159-170), captured from the culture supernatant via immobilized metal ion affinity chromatography and purified by a size exclusion chromatography.

Affinity Constants:

To determine the affinity of the antibodies to ADM, the kinetics of binding of ADM to immobilized antibody was determined by means of label-free surface plasmon resonance using a Biacore 2000 system (GE Healthcare Europe GmbH, Freiburg, Germany). Reversible immobilization of the antibodies was performed using an anti-mouse Fc antibody covalently coupled in high density to a CM5 sensor surface according to the manufacturer's instructions (mouse antibody capture kit; GE Healthcare) (Lorenz et al. 2011. Antimicrob Agents Chemother. 55(1). 165-173).

The monoclonal antibodies were raised against the below depicted ADM regions of human and murine ADM, respectively. The following table represents a selection of obtained antibodies used in further experiments. Selection was based on target region:

TABLE 1

| | | | | Affinity |
|---|---|---|---|---|
| Sequence | | ADM | | constants |
| Number | Antigen/Immunogen | Region | Designation | Kd (M) |
| SEQ ID: 4 | YRQSMNNFQGLRSFGCRFGTC | 1-21 | NT-H | 5.9 × 10⁻⁹ |
| SEQ ID: 21 | CTVQKLAHQIYQ | 21-32 | MR-H | 2 × 10⁻⁹ |
| Amidated | C-APRSKISPQGY-NH₂ | C-42-52 | CT-H | 1.1 × 10⁻⁹ |
| SEQ ID: 2 | | | | |
| (with | | | | |
| additional | | | | |
| Cysteine at | | | | |
| N-terminus | | | | |
| SEQ ID: 18 | YRQSMNQGSRSNGCRFGTC | 1-19 | NT-M | 3.9 × 10⁻⁹ |
| SEQ ID: 19 | CTFQKLAHQIYQ | 19-31 | MR-M | 4.5 × 10⁻¹⁰ |
| Amidated | C-APRNKISPQGY-NH₂ | C-40-50 | CT-M | 9 × 10⁻⁹ |
| SEQ ID: 20 | | | | |
| (with | | | | |
| additional | | | | |
| Cysteine at | | | | |
| N-terminus | | | | |

Generation of Antibody Fragments by Enzymatic Digestion:

The generation of Fab and F(ab)2 fragments was done by enzymatic digestion of the murine full length antibody NT-M. Antibody NT-M was digested using a) the pepsin-based F(ab)2 Preparation Kit (Pierce 44988) and b) the papain-based Fab Preparation Kit (Pierce 44985). The fragmentation procedures were performed according to the instructions provided by the supplier. Digestion was carried out in case of F(ab)2-fragmentation for 8 h at 37° C. The Fab-fragmentation digestion was carried out for 16 h, respectively.

Procedure for Fab Generation and Purification:

The immobilized papain was equilibrated by washing the resin with 0.5 ml of Digestion Buffer and centrifuging the column at 5000×g for 1 minute. The buffer was discarded afterwards. The desalting column was prepared by removing the storage solution and washing it with digestion buffer, centrifuging it each time afterwards at 1000×g for 2 minutes. 0.5 ml of the prepared IgG sample where added to the spin column tube containing the equilibrated Immobilized Papain. Incubation time of the digestion reaction was done for 16 h on a tabletop rocker at 37° C. The column was centrifuged at 5000×g for 1 minute to separate digest from the Immobilized Papain. Afterwards the resin was washed with 0.5 ml PBS and centrifuged at 5000×g for 1 minute. The wash fraction was added to the digested antibody that the total sample volume was 1.0 ml. The NAb Protein A Column was equilibrated with PBS and IgG Elution Buffer at room temperature. The column was centrifuged for 1 minute to remove storage solution (contains 0.02% sodium azide) and equilibrated by adding 2 ml of PBS, centrifuge again for 1 minute and the flow-through discarded. The sample was applied to the column and resuspended by inversion. Incubation was done at room temperature with end-over-end mixing for 10 minutes. The column was centrifuged for 1 minute, saving the flow-through with the Fab fragments. (References: Coulter and Harris 1983. J. Immunol. Meth. 59, 199-203.; Lindner et al. 2010. Cancer Res. 70, 277-87; Kaufmann et al. 2010. PNAS. 107, 18950-5.; Chen et al. 2010. PNAS. 107, 14727-32; Uysal et al. 2009 J. Exp. Med. 206, 449-62; Thomas et al. 2009. J. Exp. Med. 206, 1913-27; Kong et al. 2009 J. Cell Biol. 185, 1275-840).

Procedure for Generation and Purification of F(Ab′)2 Fragments:

The immobilized Pepsin was equilibrated by washing the resin with 0.5 ml of Digestion Buffer and centrifuging the column at 5000×g for 1 minute. The buffer was discarded afterwards. The desalting column was prepared by removing the storage solution and washing it with digestion buffer, centrifuging it each time afterwards at 1000×g for 2 minutes. 0.5 ml of the prepared IgG sample where added to the spin column tube containing the equilibrated Immobilized Pepsin. Incubation time of the digestion reaction was done for 16 h on a tabletop rocker at 37° C. The column was centrifuged at 5000×g for 1 minute to separate digest from the Immobilized Papain. Afterwards the resin was washed with 0.5 mL PBS and centrifuged at 5000×g for 1 minute. The wash fraction was added to the digested antibody that the total sample volume was 1.0 ml. The NAb Protein A Column was equilibrated with PBS and IgG Elution Buffer at room temperature. The column was centrifuged for 1 minute to remove storage solution (contains 0.02% sodium azide) and equilibrated by adding 2 mL of PBS, centrifuge again for 1 minute and the flow-through discarded. The sample was applied to the column and resuspended by inversion. Incubation was done at room temperature with end-over-end mixing for 10 minutes. The column was centrifuged for 1 minute, saving the flow-through with the Fab fragments. (References: Mariani et al. 1991. Mol. Immunol. 28: 69-77; Beale 1987. Exp Comp Immunol 11:287-96; Ellerson et al. 1972. FEBS Letters 24(3):318-22; Kerbel and Elliot 1983. Meth Enzymol 93:113-147; Kulkarni et al. 1985. Cancer Immunol Immunotherapy 19:211-4; Lamovi 1986. Meth Enzymol 121:652-663; Parham et al. 1982. J Immunol Meth 53:133-73; Raychaudhuri et al. 1985. Mol Immunol 22(9):1009-19; Rousseaux et al. 1980. Mol Immunol 17:469-82; Rousseaux et al. 1983. J Immunol Meth 64:141-6; Wilson et al. 1991. J Immunol Meth 138:111-9).

NT-H-Antibody Fragment Humanization:

The antibody fragment was humanized by the CDR-grafting method (Jones et al. 1986. Nature 321, 522-525).

The following steps where executed to achieve the humanized sequence:
Total RNA extraction: Total RNA was extracted from NT-H hybridomas using the Qiagen kit.
First-round RT-PCR: QIAGEN® OneStep RT-PCR Kit (Cat No. 210210) was used. RT-PCR was performed with primer sets specific for the heavy and light chains. For each RNA sample, 12 individual heavy chain and 11 light chain RT-PCR reactions were set up using degenerate forward primer mixtures covering the leader sequences of variable regions. Reverse primers are located in the constant regions of heavy and light chains. No restriction sites were engineered into the primers.
Reaction Setup: 5×QIAGEN® OneStep RT-PCR Buffer 5.0 μl, dNTP Mix (containing 10 mM of each dNTP) 0.8 μl, Primer set 0.5 μl, QIAGEN® OneStep RT-PCR Enzyme Mix 0.8 μl, Template RNA 2.0 μl, RNase-free water to 20.0 l, Total volume 20.0 μl PCR condition: Reverse transcription: 50° C., 30 min; Initial PCR activation: 95° C., 15 min Cycling: 20 cycles of 94° C., 25 sec; 54° C., 30 sec; 72° C., 30 sec; Final extension: 72° C., 10 min Second-round semi-nested PCR: The RT-PCR products from the first-round reactions were further amplified in the second-round PCR. 12 individual heavy chain and 11 light chain RT-PCR reactions were set up using semi-nested primer sets specific for antibody variable regions.
Reaction Setup: 2×PCR mix 10 μl; Primer set 2 μl; First-round PCR product 8 μl; Total volume 20 μl; Hybridoma Antibody Cloning Report PCR condition: Initial denaturing of 5 min at 95° C.; 25 cycles of 95° C. for 25 sec, 57° C. for 30 sec, 68° C. for 30 sec; Final extension is 10 min 68° C.
After PCR is finished, run PCR reaction samples onto agarose gel to visualize DNA fragments amplified. After sequencing more than 15 cloned DNA fragments amplified by nested RT-PCR, several mouse antibody heavy and light chains have been cloned and appear correct. Protein sequence alignment and CDR analysis identifies one heavy chain and one light chain. As the amino acids on positions 26, 40 and 55 in the variable heavy chain and amino acid on position 40 in the variable light are critical to the binding properties, they may be reverted to the murine original. The resulting candidates are depicted below. (Padlan 1991. Mol. Immunol. 28, 489-498; Harris and Bajorath. 1995. Protein Sci. 4, 306-310).

Annotation for the antibody fragment sequences (SEQ ID No.: 10 to 17): bold and underlined are the CDR 1, 2, 3 in numeric order from the N-terminus to the C-terminus; italic are constant regions; hinge regions are highlighted with bold, underlined letters and the histidine tag at the C-terminus with bold and italic letters.

(AM-VH-C)
SEQ ID No.: 10
QVQLQQSGAELMKPGASVKISCKATGYTFSRYWIEWVKQRPGHGLEWIGEILPGSGSTNYN
EKFKGKATITADTSSNTAYMQLSSLTSEDSAVYYC [symbol] WGQGTTLTVSSASTK
GPSVFPLAPSSKSTSGGTAALGCLVKDYFPEPVTVSWNSGALTSGVHTFPAVLQSSGLYSL
SSVVTVPSSSLGTQTYICNVNHKPSNTKVDKRVEPK [symbol]
(AM-VH1)
SEQ ID No.: 11
QVQLVQSGAEVKKPGSSVKVSCKASGYTFSRYWISWVRQAPGQGLEWMGRILPGSGSTNYA
QKFQGRVTITADESTSTAYMELSSLRSEDTAVYYC [symbol] WGQGTTVTVSSASTK
GPSVFPLAPSSKSTSGGTAALGCLVKDYFPEPVTVSWNSGALTSGVHTFPAVLQSSGLYSL
SSVVTVPSSSLGTQTYICNVNHKPSNTKVDKRVEPK [symbol]
(AM-VH2-E40)
SEQ ID No.: 12
QVQLVQSGAEVKKPGSSVKVSCKASGYTFSRYWIEWVRQAPGQGLEWMGRILPGSGSTNYA
QKFQGRVTITADESTSTAYMELSSLRSEDTAVYYC [symbol] WGQGTTVTVSSASTK
GPSVFPLAPSSKSTSGGTAALGCLVKDYFPEPVTVSWNSGALTSGVHTFPAVLQSSGLYSL
SSVVTVPSSSLGTQTYICNVNHKPSNTKVDKRVEPK [symbol]
(AM-VH3-T26-E55)
SEQ ID No.: 13
QVQLVQSGAEVKKPGSSVKVSCKATGYTFSRYWISWVRQAPGQGLEWMGEILPGSGSTNYA
QKFQGRVTITADESTSTAYMELSSLRSEDTAVYYC [symbol] WGQGTTVTVSSASTK
GPSVFPLAPSSKSTSGGTAALGCLVKDYFPEPVTVSWNSGALTSGVHTFPAVLQSSGLYSL
SSVVTVPSSSLGTQTYICNVNHKPSNTKVDKRVEPK [symbol]
(AM-VH4-T26-E40-E55)
SEQ ID No.: 14
QVQLVQSGAEVKKPGSSVKVSCKATGYTFSRYWIEWVRQAPGQGLEWMGEILPGSGSTNYA
QKFQGRVTITADESTSTAYMELSSLRSEDTAVYYC [symbol] WGQGTTVTVSSASTK
GPSVFPLAPSSKSTSGGTAALGCLVKDYFPEPVTVSWNSGALTSGVHTFPAVLQSSGLYSL
SSVVTVPSSSLGTQTYICNVNHKPSNTKVDKRVEPK [symbol]
(AM-VL-C)
SEQ ID No.: 15
DVLLSQTPLSLPVSLGDQATISCRSSQSIVYSNGNTYLEWYLQKPGQSPKLLIYRVSNRFS
GVPDRFSGSGSGTDFTLKISRVEAEDLGVYYCFQGSHIPYTFGGGTKLEIKRTVAAPSVFI
FPPSDEQLKSGTASVVCLLNNFYPREAKVQWKVDNALQSGNSQESVTEQDSKDSTYSLSST
LTLSKADYEKHKVYACEVTHQGLSSPVTKSFNRGEC
(AM-VL1)
SEQ ID No.: 16
DVVMTQSPLSLPVTLGQPASISCRSSQSIVYSNGNTYLNWFQQRPGQSPRRLIYRVSNRDS
GVPDRFSGSGSGTDFTLKISRVEAEDVGVYYCFQGSHIPYTFGQGTKLEIKRTVAAPSVFI
FPPSDEQLKSGTASVVCLLNNFYPREAKVQWKVDNALQSGNSQESVTEQDSKDSTYSLSST
LTLSKADYEKHKVYACEVTHQGLSSPVTKSFNRGEC
(AM-VL2-E40)
SEQ ID No.: 17
DVVMTQSPLSLPVTLGQPASISCRSSQSIVYSNGNTYLEWFQQRPGQSPRRLIYRVSNRDS
GVPDRFSGSGSGTDFTLKISRVEAEDVGVYYCFQGSHIPYTFGQGTKLEIKRTVAAPSVFI
FPPSDEQLKSGTASVVCLLNNFYPREAKVQWKVDNALQSGNSQESVTEQDSKDSTYSLSST
LTLSKADYEKHKVYACEVTHQGLSSPVTKSFNRGEC
(Adrecizumab heavy chain)
SEQ ID No.: 22
QVQLVQSGAEVKKPGSSVKVSCKASGYTFSRYWIEWVRQAPGQGLEWIGEILPGSGSTNYN
QKFQGRVTITADTSTSTAYMELSSLRSEDTAVYYCTEGYEYDGFDYWGQGTTVTVSSASTK
GPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPELLGGPSVFLF
PPKPKDTLMISRTPEVTCVVVDVSHEDPEVKFNWYVDGVEVHNAKTKPREEQYNSTYRVVS
VLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCS
VMHEALHNHYTQKSLSLSPGK
(Adrecizumab light chain)
SEQ ID No.: 23
DVVLTQSPLSLPVTLGQPASISCRSSQSIVYSNGNTYLEWYLQRPGQSPRLLIYRVSNRFS
GVPDRFSGSGSGTDFTLKISRVEAEDVGVYYCFQGSHIPYTFGGGTKLEIKRTVAAPSVFI
FPPSDEQLKSGTASVVCLLNNFYPREAKVQWKVDNALQSGNSQESVTEQDSKDSTYSLSST
LTLSKADYEKHKVYACEVTHQGLSSPVTKSFNRGEC
(aa 1-14 of human ADM)
SEQ ID No.: 24
YRQSMNNFQGLRSF
(aa 1-10 of human ADM)
SEQ ID No.: 25
YRQSMNNFQG

### Example 2

Methods

Design

The AdrenOSS-2 trial was a double-blind, placebo-controlled, randomized, multicenter, proof-of-concept, biomarker-guided and dose-finding phase II trial to investigate the safety, tolerability, efficacy and pharmacokinetics of Adrecizumab in patients with early septic shock and elevated bio-ADM levels. The trial was conducted in thirty hospitals with medical, surgical and/or mixed ICU in Belgium, France, Germany and the Netherlands. Further details on the trial setting have already been reported earlier by Geven et al. (Geven C, Blet A, Kox M, Hartmann O, Scigalla P, Zimmermann J, Marx G, Laterre P F, Mebazaa A, Pickkers P, (2019) A double-blind, placebo-controlled, randomised, multicentre, proof-of-concept and dose-finding phase II clinical trial to investigate the safety, tolerability and efficacy of adrecizumab in patients with septic shock and elevated adrenomedullin concentration (AdrenOSS-2). BMJ Open 0: e024475).

Ethical Considerations

The trial procedures and the Informed Consent Form (ICF) process approved by the respective independent ethics committee (IEC) followed international standards and national requirements of each participating country.

Participants

Male and female patients (>18 years) with diagnosed early septic shock (<12 h) and elevated bio-ADM values (>70 pg/mL) meeting the in- and exclusion criteria were screened upon ICU admission and start of vasopressor therapy. A list of the inclusion and exclusion criteria was published previously in Geven et al. (Geven C, Blet A, Kox M, Hartmann O, Scigalla P, Zimmermann J, Marx G, Laterre P F, Mebazaa A, Pickkers P, (2019) A double-blind, placebo-controlled, randomised, multicentre, proof-of-concept and dose-finding phase II clinical trial to investigate the safety, tolerability and efficacy of adrecizumab in patients with septic shock and elevated adrenomedullin concentration (AdrenOSS-2). BMJ Open 0: e024475).

Inclusion criteria

| | |
|---|---|
| 1. | Written informed consent by patient or legal representative |
| | (according to country - specific regulations) |
| 2. | Male and female patient, age ≥18 years |
| 3. | Body weight 50-120 kg |
| 4. | Bio-ADM concentration >70 pg/mL |
| 5. | Patient with early septic shock (start of vasopressor therapy <12 |
| | hours) |
| 6. | Women of childbearing potential must have a negative serum or urine |
| | pregnancy test before randomization and have to |
| | use a highly effective method of contraception |

Exclusion criteria

| | |
|---|---|
| 1. | Moribund and death is considered imminent <28 days |
| 2. | Pre-existing unstable condition (e.g. a recent cerebral hemorrhage or |
| | infarct, a recent acute unstable myocardial infarction (all <3 months), |
| | congestive heart failure - New York Heart Association (NYHA) |
| | Class IV |
| 3. | Patients that required cardiopulmonary resuscitation in the last 4 |
| | weeks prior to evaluation for enrollment |
| 4. | Severe Chronic Obstructive Pulmonary Disease (COPD) with |
| | chronic oxygen need at home (GOLD IV) |
| 5. | Any organ or bone marrow transplant within the past 24 weeks |
| 6. | Uncontrolled serious hemorrhage (≥2 units of blood/platelets in the |
| | previous 24 hrs.). Patients may be considered for enrollment |
| | if bleeding has stopped and patient is otherwise qualified |
| 7. | Uncontrolled hematological/oncological malignancies |
| 8. | Immunosuppressed patients (known HIV, absolute neutropenia <500 |
| | per μL) |
| 9. | Treatment with immunosuppressive drugs |
| 10. | Severe chronic liver disease (Child-Pugh C) |
| 11. | Systemic fungal infection or active tuberculosis |
| 12. | Neuromuscular disorders that impact breathing/spontaneous ventilation |
| 13. | Burns >30% of body surface |
| 14. | Plasmapheresis |
| 15. | Women who are pregnant or nursing |
| 16. | Participation in a clinical trial involving another investigational |
| | drug within 4 weeks prior to inclusion |
| 17. | Unwilling or unable to be fully evaluated for all follow-up visits |

After written ICF was provided by the patient or his/her legal representative (depending on IEC authorized local procedures) and when plasma bio-ADM concentration was >70 pg/mL (Sphingotest® bio-ADM, sphingotec GmbH, Hennigsdorf, Germany), the clinical coordination center (CCC) confirmed patient eligibility. All trial related data were captured in a pseudonymized way.

Intervention

Patients were randomly assigned in a 1:1:2 ratio to either treatment arm A (Adrecizumab 2 mg/kg), treatment arm B (Adrecizumab 4 mg/kg) or placebo. Patients received the assigned trial medication in a single intravenous infusion (duration: approximately 1 hour) within 12 hours after start of vasopressor therapy. A detailed description of the application scheme has already been published by Geven et al. [2].

Outcome Measures

Among others, the 7-, 28- and 90-day mortality rates, as well as the daily change in SOFA score relative to the SOFA score at inclusion were recorded (SOFA=Sequential Organ Failure Assessment). Another endpoint was the mean fluid balance up to 7 days after Adrecizumab/placebo infusion.

Of particular interest was the influence of time from shock diagnosis to treatment and time from ICU admission to treatment, respectively, on outcome measures.

Statistics and Data Analyses

The analyses in the present example included the comparison of the combined Adrecizumab doses to placebo. The per-protocol (PP) analysis included all patients who received the trial medication according to the protocol with minor deviations only and satisfied all major entry criteria. The patient population was further enriched by excluding patients with a circulating Dipeptidyl-Peptidase 3 (DPP3) concentration above 50 ng/mL.

Continuous data were analyzed by number of patients, arithmetic mean, standard deviation (SD) or median with interquartile range, as appropriate, and exploratory comparisons between treatment arms and placebo were done using the Kruskal-Wallis test. Categorial variables are summarized category-wise giving numbers and percentages, and compared using the Chi² test for contingency tables. Both treatment arms (2 mg/kg and 4 mg/kg) were combined for the efficacy analyses. The log-rank test was chosen for showing differences in mortality rates among treatment groups, and Kaplan-Meier plots were used for illustration. Unadjusted and adjusted hazard ratios (HR) were estimated via Cox Proportional Hazard models. Missing SOFA scores due to death or discharge from ICU were imputed (setting them to either 24 or 0, respectively, as e.g. proposed by de Grooth et al. (de Grooth H J, Geenen I L, Girbes A R, Vincent J L, Parienti J J, Oudemans-van Straaten H M, (2017) SOFA and mortality endpoints in randomized controlled trials: a systematic review and meta-regression analysis. Critical care 21: 38)). All reported p-values are 2-sided. P-values below 0.05 were considered significant. Statistical analyses were performed using SAS version 9.3, and R version 3.4.3 (http://www.r-project.org).

Patients and Study Treatment

The first patient was randomized on Dec. 8, 2017. The last patient was enrolled on Sep. 25, 2019. Altogether, 459 patients were screened. Out of these, 158 were not-eligible and therefore not randomized (n=91 with bio-ADM<70 pg/mL; n=67 for not meeting inclusion/exclusion criteria). In total 301 patients were randomized in four countries to either placebo (n=152), 2 mg/kg (n=72) or 4 mg/kg of Adrecizumab (n=77), defining the intention to treat (ITT) population. For the per protocol analysis (PP) n=7 patients were excluded. Finally, n=50 patients were excluded who had pre-dose levels of >50 ng/mL of DPP3, so that a total patient population of n=244 was analyzed. Elevated plasma concentrations of DPP3 trigger a detrimental pathway, which is mechanistically different from the ADM pathway and therefore cannot be addressed by Adrecizumab.

Results

One important aspect in narrowing down the patient population, which benefits most of a drug, is to define the treatment window relative to the particular clinical condition of patients. This is especially important in acutely changing and life-threatening conditions such as sepsis, septic shock or other acute circulatory disorders. We investigated the efficacy of Adrecizumab as a function of disease progression in septic shock. Operationally, disease progression translates into the change of requirement of organ support, time since septic shot onset, time since ICU admission.

For the assessment of the issue described above, we studied sub-populations of the AdrenOSS-2 clinical trial.

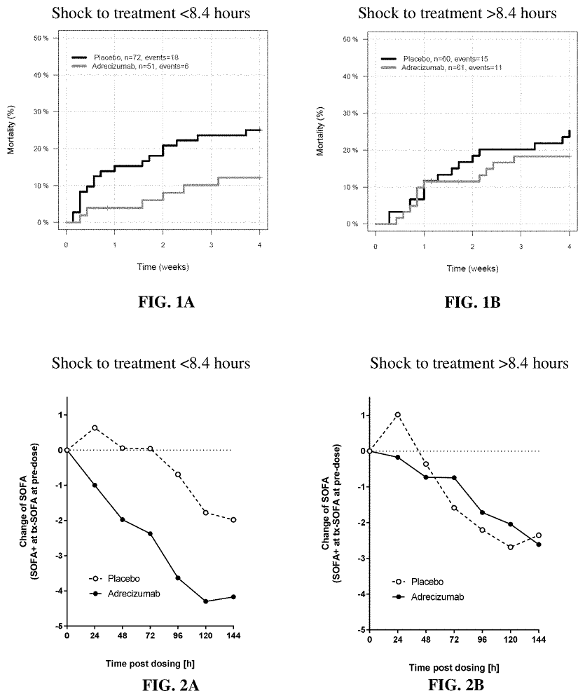

To investigate the impact of the length of time between shock diagnosis and the start of treatment with Adrecizumab, the population was split in two groups at the median of the length of time between shock diagnosis and the start of treatment, namely 8.4 hours, and both groups were compared. The 28-day mortality rate was lower with Adrecizumab treatment compared to placebo when the treatment was administered between 0 and 10 hours after shock was diagnosed (HR 0.439 (0.174-1.11)) (FIG. 1A). When the time between shock diagnosis and treatment was between 10 and 12.2 hours the difference in mortality rate between Adrecizumab treatment compared to placebo was less pronounced (HR 0.711 (0.326-1.55)) (FIG. 1B).

FIG. 2 shows the changes in SOFA Score when Adrecizumab treatment was administered within 8.4 h after shock diagnosis (FIG. 2A) compared to treatment later than 8.4 h after shock diagnosis (FIG. 2B) supporting the surprisingly beneficial effect of the antibody of the present invention.

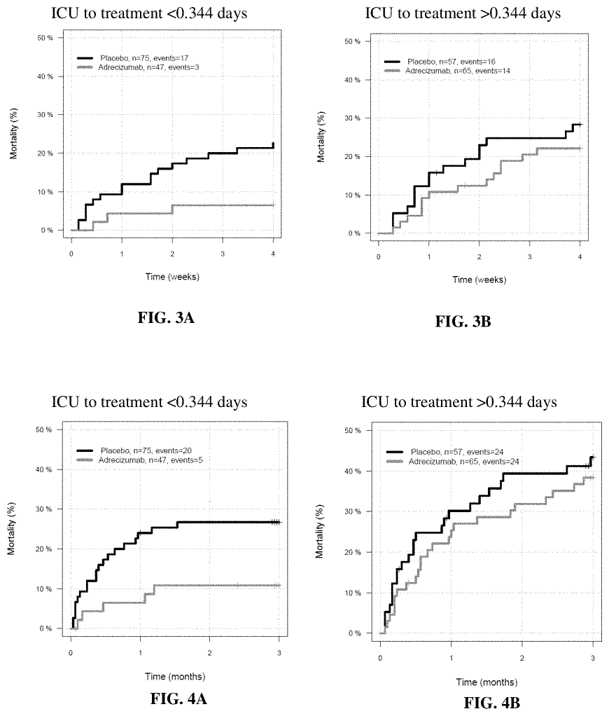

To investigate the impact of the length of time between patients' admission at the ICU and the start of treatment with Adrecizumab, the population was split in two groups at the median of the length of time between patients' admission at the ICU and the start of treatment, namely 0.344 days (8.3 hours), and both groups were compared. The 28-day mortality rate was significantly lower with Adrecizumab treatment compared to placebo when the treatment was administered between 0 and 0.344 days (8.3 hours) after ICU admission (HR 0.263 (0.077-0.898), log rank p-value 0.022) (FIG. 3A). When the time between ICU admission and treatment was between 0.344 days and 29 days the difference in mortality between Adrecizumab treatment compared to placebo was less pronounced (HR 0.734 (0.358-1.50)) (FIG. 3B). The overproportionally beneficial treatment effect in the group of patients, who received treatment no later than 0.344 days (8.3 hours) after ICU admission pertained through the entire observation period of 90-days: The 90-day mortality rate was significantly lower with Adrecizumab treatment compared to placebo when the treatment was administered between 0 and 0.344 days (8.3 hours) after ICU admission (HR 0.364 (0.137-0.970), log rank p-value 0.035) (FIG. 4A). When the time between ICU admission and treatment was between 0.344 days and 29 days the difference in mortality between Adrecizumab treatment compared to placebo was less pronounced (HR 0.826 (0.469-1.45)) (FIG. 4B).

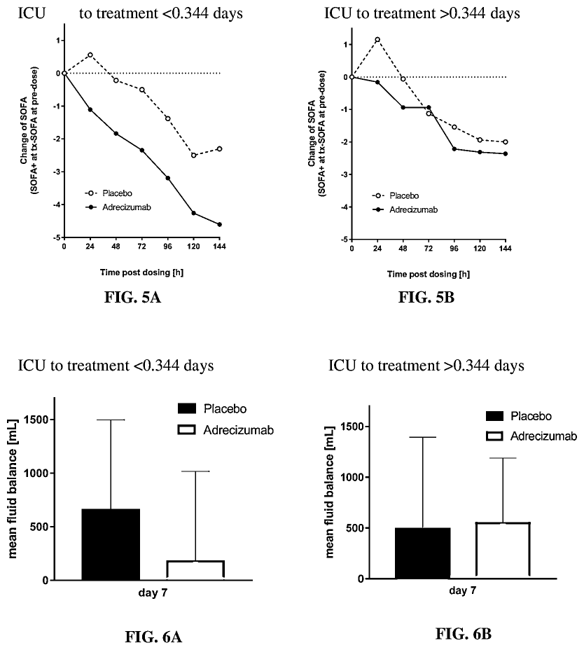

FIG. 5 shows the changes in SOFA Score when Adrecizumab treatment was administered 0.344 days after ICU admission (FIG. 5A) compared to treatment more than 0.344 days post ICU admission (FIG. 5B). The SOFA score was rapidly and sustainably reduced, when Adrecizumab treatment was administered within 0.344 days after ICU admission, supporting the surprisingly beneficial effect of the antibody of the present invention.

FIG. 6 shows the mean fluid balance on day 7 after start of treatment, when Adrecizumab treatment was administered within 0.344 days after ICU admission (FIG. 6A) compared to treatment administered more than 0.344 days post ICU admission (FIG. 6B). The fluid balance (fluid input minus fluid output) was recorded for each 24 h period on the ICU. The mean fluid balance up to 7 days after Adrecizumab/placebo infusion was calculated per patient. Days with missing fluid balance, e.g. due to discharge from the ICU or death, were not considered. Fluid balance was significantly reduced by 70.2%, when Adrecizumab treatment was administered within 0.344 days after ICU admission, but it was not reduced when treatment was administered later after ICU admission, supporting the surprisingly beneficial effect of the antibody of the present invention.

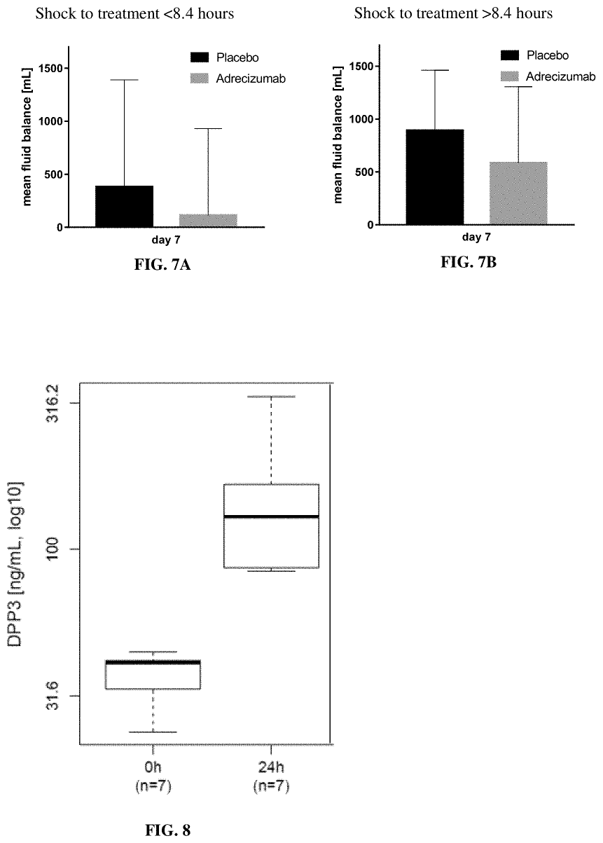

FIG. 7 shows the mean fluid balance on day 7 after start of treatment, when Adrecizumab treatment was administered within 8.4 hours after shock diagnosis (FIG. 8A) compared to treatment administered more than 8.4 hours after shock diagnosis (FIG. 8B). The fluid balance (fluid input minus fluid output) was recorded for each 24 h period on the ICU. The mean fluid balance up to 7 days after Adrecizumab/placebo infusion was calculated per patient. Days with missing fluid balance, e.g. due to discharge from the ICU or death, were not considered. Fluid balance was reduced by 58.2%, when Adrecizumab treatment was administered within 10 hours after shock diagnosis, but it much less reduced (30.4%) when treatment was administered later after shock diagnosis, supporting the surprisingly beneficial effect of the antibody of the present invention.

Patients who received Adrecizumab or Placebo infusion early after shock diagnosis or ICU admission exhibited a more severe clinical condition at the time of Adrecizumab or Placebo infusion start than those treated later, as indicated by a higher APACHE II score and other severity-associated variables. This was expected, as standard of care therapeutic measures usually lead to a short-term, albeit not sustained improvement of the patients' clinical condition.

Comparison of patients who received Adrecizumab with those who received placebo did not reveal relevant differences regarding their baseline characteristics, so that the observed beneficial treatment effect of Adrecizumab is not confounded by other factors.

The beneficial treatment effect of Adrecizumab was more pronounced the earlier the treatment was initiated. In an exemplary analysis, the population was split in three groups after ranking the patients depending on the time from ICU admission till start of treatment. The 28-day mortality rate was analyzed for the first, second and a combination of the third and fourth quartile of this population. Quartile 1 covered the time frame of 0-0.24 days after ICU admission, quartile 2 covered the time frame of 0.25-0.34 days after ICU admission, and quartiles 3/4 covered the time frame of >0.35 days after ICU admission. The mortality rate was reduced most pronounced in quartile 1 (85%), clearly detectable but less pronounced in quartile 2 (53%), and least pronounced but still evident in quartiles 3/4 (23%) (Table 2).

TABLE 2
Mortality rate reduction by Adrecizumab depending on the time from ICU admission to start
of treatment. Patients were ranked by the time from ICU admission till start of treatment and
split in three groups (quartiles 1, 2 and 3 + 4). 28-day mortality data are shown.

| | Q1 (0-0.24 days) | | Q2 (0.25-0.34 days) | | Q3/Q4 >0.35 days | |
|---|---|---|---|---|---|---|
| Follow-up | 28 days | | 28 days | | 28 days | |
| Treatment | Placebo | Adrecizumab | Placebo | Adrecizumab | Placebo | Adrecizumab |
| dead (n) | 10 | 1 | 7 | 2 | 16 | 14 |
| all (n) | 37 | 24 | 38 | 23 | 57 | 65 |
| Mortality rate | 27% | 4% | 18% | 9% | 28% | 22% |
| Mortality rate | 85% | | 53% | | 23% | |
| reduction by | | | | | | |
| Adrecizumab relative | | | | | | |
| to Placebo | | | | | | |

### Example 3

From the AdrenOSS-1 trial, septic shock patients with ICU admission bio-ADM concentrations above 70 pg/mL and DPP3 concentrations below 50 ng/mL were selected, and those further analyzed (n=7), which exhibited an increase of DPP3 above 70 ng/mL on the next day. The median concentration increased from 41.0 ng/mL to 129.7 ng/mL (see FIG. 8) and Table 3.

TABLE 3
DPP3 concentrations from septic shock patients on the day of ICU admission and on the following day

| Subgroup | Mean | SD | Median | IOR | Q1 | Q3 | Q.05 | Q.95 | Q.975 | Q.99 | Min | Max | n |
|---|---|---|---|---|---|---|---|---|---|---|---|---|---|
| 0 h | 37.126 | 7.879 | 41.000 | 8.0 | 33.647 | 41.647 | 25.1 | 43.8 | 44.2 | 44.4 | 23.8 | 44.5 | 7 |
| 24 h | 151.479 | 90.005 | 129.706 | 82.9 | 86.733 | 169.647 | 84.9 | 292.4 | 313.0 | 325.3 | 84.4 | 333.5 | 7 |

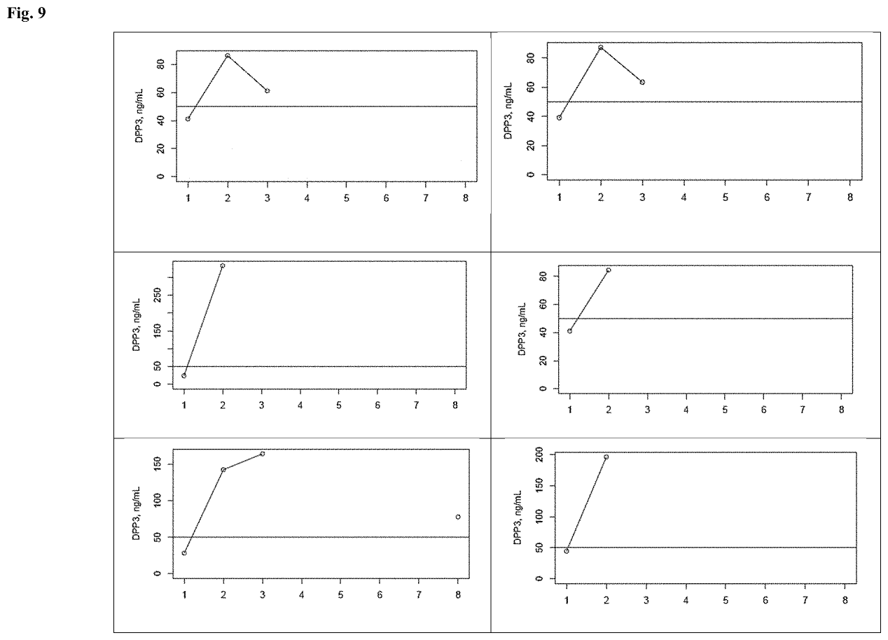

This is further exemplified in individual patient courses on FIG. 9.

The data demonstrate that septic shock patients treatable with Adrecizumab on ICU admission can develop pathologically elevated plasma levels of DPP3. These in turn can induce shock and death, as described in the literature (Blet et al., Crit Care. 2021 Feb. 15; 25(1):61). Thus, this supports the concept to initiate treatment of septic shock patients with Adrecizumab early, rather than late, in order to avoid fatal developments such as increase of DPP3 and subsequent shock and death.

1. A method for therapy of a patent with shock, comprising administering anti-adrenomedullin (ADM) antibody or an anti-adrenomedullin antibody fragment or anti-ADM non-Ig scaffold to a patient with shock, in particular septic shock, wherein said patient:
has suffered from shock, in particular from a septic shock not longer than 10 hours at the starting point of treatment with said Anti-adrenomedullin (ADM) antibody or an anti-adrenomedullin antibody fragment or anti-ADM non-Ig scaffold and/or
has been admitted to ICU not longer than 10 hours at the starting point of treatment with said Anti-adrenomedullin (ADM) antibody or an anti-adrenomedullin antibody fragment or anti-ADM non-Ig scaffold, and/or
has not received organ support at all or not longer than 10 hours of organ support at the starting point of treatment with said Anti-adrenomedullin (ADM) antibody or an anti-adrenomedullin antibody fragment or anti-ADM non-Ig scaffold, and
wherein said antibody or fragment or scaffold binds to the N-terminal part (aa 1-21) of ADM:

| (SEQ ID NO. 4) | |
|---|---|
| | YRQSMNNFQGLRSFGCRFGTC. |

2. The method for therapy according to claim 1, wherein said patient has suffered from shock, in particular from a septic shock, not longer than 9, preferably 8.4, preferably 8.26 (0.344 days), preferably 8, preferably 7, preferably 6, preferably 5.76 (0.25 days), preferably 5.75 (0.24 days), 5. preferably 4, preferably 3 hours at the starting point of treatment with said Anti-adrenomedullin (ADM) antibody or an anti-adrenomedullin antibody fragment or anti-ADM non-Ig scaffold.

3. The method for therapy according to claim 1, wherein said patient has been admitted to ICU not longer than 9, preferably 8.4, preferably 8.26 (0.344 days), preferably 8, preferably 7, preferably 6, preferably 5.76 (0.25 days), preferably 5.75 (0.24 days) preferably 5. preferably 4, preferably 3 hours at the starting point of treatment with said Anti-adrenomedullin (ADM) antibody or an anti-adrenomedullin antibody fragment or anti-ADM non-Ig scaffold.

4. The method for therapy according to claim 1, wherein said patient has received organ support not longer than 9, preferably 8.4, preferably 8.26 (0.344 days), preferably 8, preferably 7, preferably 6, preferably 5.76 (0.25 days), preferably 5.75 (0.24 days), preferably 5. preferably 4, preferably 3 hours at the starting point of treatment with said Anti-adrenomedullin (ADM) antibody or an anti-adrenomedullin antibody fragment or anti-ADM non-Ig scaffold

5. A method for therapy of a patient suffering from shock, in particular septic shock, wherein said Anti-adrenomedullin (ADM) antibody or an anti-adrenomedullin antibody fragment or anti-ADM non-Ig scaffold is administered
within 10 hours after occurrence of shock in said patient and/or
within 10 hours after admission of said patient to ICU, and/or
before the patient has received organ support or within not longer than 10 hours of organ support, and
wherein said antibody or fragment or scaffold binds to the N-terminal part (aa 1-21) of ADM:

| (SEQ ID NO. 4) | |
|---|---|
| | YRQSMNNFQGLRSFGCRFGTC. |

6. The method for therapy of a patient suffering from shock, in particular septic shock according to claim 5 antibody or an anti-adrenomedullin antibody fragment or anti-ADM non-Ig scaffold is administered within 9, preferably 8.4, preferably 8.26 (0.344 days), preferably 8, preferably 7, preferably 6, preferably 5.76 (0.25 days), preferably 5.75 (0.25 days), preferably 5. preferably 4, preferably 3 hours after occurrence of shock in said patient

7. The method for therapy of a patient suffering from shock, in particular septic shock according to claim 5, wherein said Anti-adrenomedullin (ADM) antibody or an anti-adrenomedullin antibody fragment or anti-ADM non-Ig scaffold is administered within 9, preferably 8.4, preferably 8.26 (0.344 days), preferably 8, preferably 7, preferably 6, preferably 5.76 (0.25 days), preferably 5.75 (0.25 days), preferably 5. preferably 4, preferably 3 hours after admission of said patient to ICU.

8. The method of therapy of a patient suffering from shock, in particular septic shock according to claim 5, wherein said anti-adrenomedullin (ADM) antibody or an anti-adrenomedullin antibody fragment or anti-ADM non-Ig scaffold is administered within 9, preferably 8.4, preferably 8.26 (0.344 days), preferably 8, preferably 7, preferably 6, preferably 5.76 (0.25 days), preferably 5.75 (0.25 days), preferably 5. preferably 4, preferably 3 hours after the patient has received organ support at the starting point of treatment with said Anti-adrenomedullin (ADM) antibody or an anti-adrenomedullin antibody fragment or anti-ADM non-Ig scaffold.

9. The method of therapy according to claim 1, wherein said patient has shock that is selected from the group comprising shock due to hypovolemia, cardiogenic shock, obstructive shock and distributive shock, in particular cardiogenic shock, septic shock, shock due to Covid-19, shock due to burns and traumatic shock.

10. The method of therapy according to claim 9, wherein said patient has septic shock.

11. The method of therapy of a patient suffering from shock, in particular septic shock, according to claim 1, wherein a sample of bodily fluid taken said patient exhibits a level of bioADM>70 pg/mL, and wherein said bodily fluid is selected from the group comprising whole blood, plasma or serum.

12. The method of therapy of a patient suffering from shock, in particular septic shock according to claim 1, wherein a sample of bodily fluid taken said patient exhibits a level of DPP3<50 ng/mL, and wherein said bodily fluid is selected from the group comprising whole blood, plasma or serum.

13. The method of therapy according to claim 1, wherein said antibody or antibody fragment or non-Ig scaffold is monospecific.

14. The method of therapy according to claim 1, wherein said antibody or fragment or scaffold exhibits a binding affinity to ADM of at least 10-7 M by label-free surface plasmon resonance using a Biacore 2000 system.

15. The method of therapy according to claim 14, wherein antibody or fragment or scaffold exhibits a binding affinity to ADM exhibits a binding affinity to ADM of between 1×10−9 to 3×10−9 by label-free surface plasmon resonance using a Biacore 2000 system.

16. The method of therapy or prevention of shock in a patient according to claim 13, wherein the anti-ADM antibody or anti-ADM antibody fragment or anti-ADM non-Ig scaffold is an IgG1 antibody.

17. The method of claim 1, wherein said antibody or fragment or scaffold is not ADM-binding-Protein-1 (complement factor H).

18. The method of therapy according to claim 1, wherein said antibody or fragment or scaffold recognizes and binds to the N-terminal end (aa 1) of ADM.

19. The method of therapy according to claim 1, wherein said antibody or fragment or scaffold is an ADM stabilizing antibody or fragment or scaffold that enhances the half-life (t½ half retention time) of ADM in serum, blood, plasma at least 10%, preferably at least, 50%, more preferably >50%, most preferably >100%.

20. The method of therapy according to claim 1, wherein said antibody or fragment or scaffold blocks the bioactivity of ADM not more than 80%, preferably not more than 50% using hADM 22-52 as a reference antagonist in CHO-K1 cells expressing human recombinant ADM receptor.

21. The method of therapy according to claim 1, wherein said subjects undergoes chemotherapy, vasopressors, treatment with biologics, antibiotics, or treatment with anti-viral compounds.

22. The method of therapy according to claim 1, wherein said antibody or fragment is a human monoclonal antibody or fragment that binds to ADM or an antibody fragment thereof wherein the heavy chain comprises the sequences:

| | CDR1: |
|---|---|
| SEQ ID NO: 5 | |
| | GYTFSRYW |
| | CDR2: |
| SEQ ID NO: 6 | |
| | ILPGSGST |
| | CDR3: |
| SEQ ID NO: 7 | |
| | TEGYEYDGFDY |

and wherein the light chain comprises the sequences:

| | CDR1: |
|---|---|
| SEQ ID NO: 8 | |
| | QSIVYSNGNTY |
| | CDR2: |
| | RVS |
| | CDR3: |
| SEQ ID NO: 9 | |
| | FQGSHIPYT. |

23. The method of therapy according to claim 22, wherein said antibody or fragment comprises a sequence selected from the group comprising as a VH region:
(AM-VH-C)
SEQ ID NO: 10
QVQLQQSGAELMKPGASVKISCKATGYTFSRYWIEWVKQRPGHGLEWIGE
ILPGSGSTNYNEKFKGKATITADTSSNTAYMQLSSLTSEDSAVYYCTEGY
EYDGFDYWGQGTTLTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKHHHHHH
(AM-VH1)
SEQ ID NO: 11
QVQLVQSGAEVKKPGSSVKVSCKASGYTFSRYWISWVRQAPGQGLEWMGR
ILPGSGSTNYAQKFQGRVTITADESTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKHHHHHH
(AM-VH2-E40)
SEQ ID NO: 12
QVQLVQSGAEVKKPGSSVKVSCKASGYTFSRYWIEWVRQAPGQGLEWMGR
ILPGSGSTNYAQKFQGRVTITADESTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKHHHHHH
(AM-VH3-T26-E55)
SEQ ID NO: 13
QVQLVQSGAEVKKPGSSVKVSCKATGYTFSRYWISWVRQAPGQGLEWMGE
ILPGSGSTNYAQKFQGRVTITADESTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKHHHHHH
(AM-VH4-T26-E40-E55)
SEQ ID NO: 14
QVQLVQSGAEVKKPGSSVKVSCKATGYTFSRYWIEWVRQAPGQGLEWMGE
ILPGSGSTNYAQKFQGRVTITADESTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKHHHHHH
and comprises a sequence selected from the group comprising the following sequence as a VL region:
(AM-VL-C)
SEQ ID NO: 15
DVLLSQTPLSLPVSLGDQATISCRSSQSIVYSNGNTYLEWYLQKPGQSPK
LLIYRVSNRFSGVPDRFSGSGSGTDFTLKISRVEAEDLGVYYCFQGSHIP
YTFGGGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
(AM-VL1)
SEQ ID NO: 16
DVVMTQSPLSLPVTLGQPASISCRSSQSIVYSNGNTYLNWFQQRPGQSPR
RLIYRVSNRDSGVPDRFSGSGSGTDFTLKISRVEAEDVGVYYCFQGSHIP
YTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
(AM-VL2-E40)
SEQ ID NO: 17
DVVMTQSPLSLPVTLGQPASISCRSSQSIVYSNGNTYLEWFQQRPGQSPR
RLIYRVSNRDSGVPDRFSGSGSGTDFTLKISRVEAEDVGVYYCFQGSHIP
YTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC.

24. The method of therapy according to claim 22, wherein said antibody or fragment comprises the following sequence as a heavy chain:
SEQ ID NO: 22
QVQLVQSGAEVKKPGSSVKVSCKASGYTFSRYWIEWVRQAPGQGLEWIGE
ILPGSGSTNYNQKFQGRVTITADTSTSTAYMELSSLRSEDTAVYYCTEGY
EYDGFDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK
or a sequence that is >95% identical to it,
and comprises the following sequence as a light chain:
SEQ ID NO: 23
DVVLTQSPLSLPVTLGQPASISCRSSQSIVYSNGNTYLEWYLQRPGQSPR
LLIYRVSNRFSGVPDRFSGSGSGTDFTLKISRVEAEDVGVYYCFQGSHIP
YTFGGGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
or a sequence that is >95% identical to it.

25. The method of therapy or prevention of shock in a patient according to claim 1, wherein the anti-adrenomedullin (ADM) antibody or anti-ADM antibody fragment or anti-ADM non-Ig scaffold binds to the N-terminal part (amino acid 1-10) of ADM: YRQSMNNFQG (SEQ ID No. 25).

26. (canceled)


SEQUENCE LISTING (25 sequences)

SEQ ID NO: 1 - protein, 52 aa, Homo sapiens
YRQSMNNFQGLRSFGCRFGTCTVQKLAHQIYQFTDKDKDNVAPRSKISPQGY

SEQ ID NO: 2 - protein, 11 aa, Homo sapiens
APRSKISPQGY

SEQ ID NO: 3 - protein, 22 aa, Homo sapiens
CTVQKLAHQIYQFTDKDKDNVA

SEQ ID NO: 4 - protein, 21 aa, Homo sapiens
YRQSMNNFQGLRSFGCRFGTC

SEQ ID NO: 5 - protein, 8 aa, Mus musculus
GYTFSRYW

SEQ ID NO: 6 - protein, 8 aa, Mus musculus
ILPGSGST

SEQ ID NO: 7 - protein, 11 aa, Mus musculus
TEGYEYDGFDY

SEQ ID NO: 8 - protein, 11 aa, Mus musculus
QSIVYSNGNTY

SEQ ID NO: 9 - protein, 9 aa, Mus musculus
FQGSHIPYT

SEQ ID NO: 10 - protein, 225 aa, Artificial Sequence
  AM-VH-C
QVQLQQSGAELMKPGASVKISCKATGYTFSRYWIEWVKQRPGHGLEWIGEILPGSGSTNYNEKFKGKATITADTSSNTAYMQLSSLTSEDSAVYYCTEGYEYDGFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKHHHHHH

SEQ ID NO: 11 - protein, 225 aa, Artificial Sequence
  AM-VH1
QVQLVQSGAEVKKPGSSVKVSCKASGYTFSRYWISWVRQAPGQGLEWMGRILPGSGSTNYAQKFQGRVTITADESTSTAYMELSSLRSEDTAVYYCTEGYEYDGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKHHHHHH

SEQ ID NO: 12 - protein, 225 aa, Artificial Sequence
  AM-VH2-E40
QVQLVQSGAEVKKPGSSVKVSCKASGYTFSRYWIEWVRQAPGQGLEWMGRILPGSGSTNYAQKFQGRVTITADESTSTAYMELSSLRSEDTAVYYCTEGYEYDGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKHHHHHH

SEQ ID NO: 13 - protein, 225 aa, Artificial Sequence
  AM-VH3-T26-E55
QVQLVQSGAEVKKPGSSVKVSCKATGYTFSRYWISWVRQAPGQGLEWMGEILPGSGSTNYAQKFQGRVTITADESTSTAYMELSSLRSEDTAVYYCTEGYEYDGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKHHHHHH

SEQ ID NO: 14 - protein, 225 aa, Artificial Sequence
  AM-VH4-T26-E40-E55
QVQLVQSGAEVKKPGSSVKVSCKATGYTFSRYWIEWVRQAPGQGLEWMGEILPGSGSTNYAQKFQGRVTITADESTSTAYMELSSLRSEDTAVYYCTEGYEYDGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKHHHHHH

SEQ ID NO: 15 - protein, 219 aa, Artificial Sequence
  AM-VL-C
DVLLSQTPLSLPVSLGDQATISCRSSQSIVYSNGNTYLEWYLQKPGQSPKLLIYRVSNRFSGVPDRFSGSGSGTDFTLKISRVEAEDLGVYYCFQGSHIPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 219 aa, Artificial Sequence
  AM-VL1
DVVMTQSPLSLPVTLGQPASISCRSSQSIVYSNGNTYLNWFQQRPGQSPRRLIYRVSNRDSGVPDRFSGSGSGTDFTLKISRVEAEDVGVYYCFQGSHIPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 219 aa, Artificial Sequence
  AM-VL2-E40
DVVMTQSPLSLPVTLGQPASISCRSSQSIVYSNGNTYLEWFQQRPGQSPRRLIYRVSNRDSGVPDRFSGSGSGTDFTLKISRVEAEDVGVYYCFQGSHIPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 19 aa, Mus musculus
YRQSMNQGSRSNGCRFGTC

SEQ ID NO: 19 - protein, 12 aa, Mus musculus
CTFQKLAHQIYQ

SEQ ID NO: 20 - protein, 11 aa, Mus musculus
APRNKISPQGY

SEQ ID NO: 21 - protein, 12 aa, Homo sapiens
CTVQKLAHQIYQ

SEQ ID NO: 22 - protein, 448 aa, Artificial Sequence
  Human antibody heavy chain with grafted mouse CDR
QVQLVQSGAEVKKPGSSVKVSCKASGYTFSRYWIEWVRQAPGQGLEWIGEILPGSGSTNYNQKFQGRVTITADTSTSTAYMELSSLRSEDTAVYYCTEGYEYDGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 219 aa, Artificial Sequence
  Human antibody light chain with grafted mouse CDRs
DVVLTQSPLSLPVTLGQPASISCRSSQSIVYSNGNTYLEWYLQRPGQSPRLLIYRVSNRFSGVPDRFSGSGSGTDFTLKISRVEAEDVGVYYCFQGSHIPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 14 aa, homo sapiens
YRQSMNNFQGLRSF

SEQ ID NO: 25 - protein, 10 aa, Homo sapiens
YRQSMNNFQG